(19) United States
(12) Patent Application Publication
(10) Pub. No.: US 2005/0037381 A1
(43) Pub. Date: Feb. 17, 2005

(54) Artificial receptors, building blocks, and methods

(75) Inventor: Robert E. Carlson, Minnetonka, MN (US)

Correspondence Address: MERCHANT & GOULD PC, P.O. BOX 2903, MINNEAPOLIS, MN, 55402-0903, US

(73) Assignee: RECEPTORS LLC, CHASKA, MN (US)

(21) Appl. No.: 10/813,568

(22) Filed: Mar. 29, 2004

Related U.S. Application Data
(63) Continuation-in-part of application No. 10/244,727, filed on Sep. 16, 2002.
(63) Continuation-in-part of application No. PCT/US03/05328, filed on Feb. 19, 2003.
(60) Provisional application No. 60/459,062, filed on Mar. 28, 2003.
(60) Provisional application No. 60/499,776, filed on Sep. 3, 2003.
(60) Provisional application No. 60/499,975, filed on Sep. 3, 2003.
(60) Provisional application No. 60/500,081, filed on Sep. 3, 2003.
(60) Provisional application No. 60/526,511, filed on Dec. 2, 2003.

(51) Int. Cl. C12Q 1/68; C12M 1/34; B05D 3/00

(52) U.S. Cl. 435/6; 427/2.11; 435/287.2

(57) ABSTRACT

The present invention relates to artificial receptors and arrays or microarrays of artificial receptors or candidate artificial receptors. Each member of the array includes a plurality of building block compounds, which can be immobilized in a spot on a support. The present invention also includes the building blocks, combinations of building blocks, arrays of building blocks, and receptors constructed of these building blocks together with a support. The present invention also includes methods of making and using these arrays and receptors.

### CROSS REFERENCE TO RELATED APPLICATIONS

The present application is a continuation in part of U.S. patent application Ser. No. 10/244,727, filed Sep. 16, 2002, and of Application No. PCT/US03/05328, filed Feb. 19, 2003, both entitled “ARTIFICIAL RECEPTORS, BUILDING BLOCKS, AND METHODS”. The present application claims priority to the fullest extent to U.S. Provisional Patent Application Ser. No. 60/459,062, filed Mar. 28, 2003; 60/499,776, 60/499,975, and 60/500,081, each filed Sep. 3, 2003; and 60/526,511, filed Dec. 2, 2003. The disclosures of each of these applications are incorporated herein by reference.

### INTRODUCTION

The present invention relates to artificial receptors, to methods and compositions for making them, and to methods using them. A receptor provides a binding site for and binds a ligand. For example, at an elementary level, receptors are often visualized having a binding site represented as a lock or site into which a key or ligand fits. The binding site is lined with, for example, hydrophobic or functional groups that provide favorable interactions with the ligand.

The present invention provides compositions and methods for developing molecules that provide favorable interactions with a selected ligand. The present compositions and methods generate a wide variety of molecular structures, one or more of which interacts favorably with the selected ligand. Heterogeneous and immobilized combinations of building block molecules form the variety of molecular structures. For example, combinations of 2, 3, 4, or 5 distinct building block molecules immobilized near one another on a support provide molecular structures that serve as candidate and working artificial receptors. FIG. 1 schematically illustrates an embodiment employing 4 distinct building blocks in a spot on a microarray to make a ligand binding site. This Figure illustrates a group of 4 building blocks at the corners of a square forming a unit cell. A group of four building blocks can be envisioned as the vertices on any quadrilateral. FIG. 1 illustrates that spots or regions of building blocks can be envisioned as multiple unit cells, in this illustration square unit cells. Groups of unit cells of four building blocks in the shape of other quadrilaterals can also be formed on a support.

Each immobilized building block molecule can provide one or more “arms” extending from a “framework” and each can include groups that interact with a ligand or with portions of another immobilized building block. FIG. 2 illustrates that combinations of four building blocks, each including a framework with two arms (called “recognition elements”), provides a molecular configuration of building blocks that form a site for binding a ligand. Such a site formed by building blocks such as those exemplified below can bind a small molecule, such as a drug, metabolite, pollutant, or the like, and/or can bind a larger ligand such as a macromolecule or microbe.

### BACKGROUND

The preparation of artificial receptors that bind ligands like proteins, peptides, carbohydrates, microbes, pollutants, pharmaceuticals, and the like with high sensitivity and specificity is an active area of research. None of the conventional approaches has been particularly successful; achieving only modest sensitivity and specificity mainly due to low binding affinity.

Antibodies, enzymes, and natural receptors generally have binding constants in the 10⁸-10¹² range, which results in both nanomolar sensitivity and targeted specificity. By contrast, conventional artificial receptors typically have binding constants of about 10³ to 10⁵, with the predictable result of millimolar sensitivity and limited specificity.

Several conventional approaches are being pursued in attempts to achieve highly sensitive and specific artificial receptors. These approaches include, for example, affinity isolation, molecular imprinting, and rational and/or combinatorial design and synthesis of synthetic or semi-synthetic receptors.

Such rational or combinatorial approaches have been limited by the relatively small number of receptors which are evaluated and/or by their reliance on a design strategy which focuses on only one building block, the homogeneous design strategy. Common combinatorial approaches form microarrays that include 10,000 or 100,000 distinct spots on a standard microscope slide. However, such conventional methods for combinatorial synthesis provide a single molecule per spot. Employing a single building block in each spot provides only a single possible receptor per spot. Synthesis of thousands of building blocks would be required to make thousands of possible receptors.

Further, these conventional approaches are hampered by the currently limited understanding of the principals which lead to efficient binding and the large number of possible structures for receptors, which makes such an approach problematic.

There remains a need for methods and materials for making artificial receptors that combines the efficiency of targeted synthesis, the spatial resolution of microarrays, and the exponential power of combinatorial display.

### SUMMARY

The present invention relates to artificial receptors, arrays of artificial receptors (e.g., candidate artificial receptors), and methods of making them. Each member of the array includes a plurality of building block compounds, which can be immobilized in a spot on a support. The present invention also includes the building blocks, combinations of building blocks, arrays of building blocks, and receptors constructed of these building blocks together with a support. The present invention also includes methods of using these arrays and receptors.

The present invention includes and employs combinations of small, selected groups of building blocks in a combinatorial microarray display format to provide candidate artificial receptors. In an embodiment, the present invention employs up to about 4 building blocks to make a candidate artificial receptor. Combinations of these building blocks can be positioned on a substrate in configurations suitable for binding ligands such as proteins, peptides, carbohydrates, pollutants, pharmaceuticals, nerve agents, toxic chemical agents, microbes, and the like.

The present artificial receptors can be prepared by methods including both focused combinatorial synthesis and targeted screening arrays. The present compositions and methods can combine the advantages of receptor focused synthesis and high throughput evaluation to rapidly identify and produce practical, target specific artificial receptors.

In an embodiment, the present invention includes a method of making a heterogeneous building block array. This method includes forming a plurality of spots on a solid support, the spots including a plurality of building blocks, and coupling a plurality of building blocks to the solid support in the spots.

In an embodiment, the present invention includes a method of using an artificial receptor. This method includes contacting a heterogeneous building block array with a test ligand, detecting binding of a test ligand to one or more spots in the array, and selecting one or more of the binding spots as the artificial receptor. The artificial receptor can be a lead or working artificial receptor. The method can also include testing a plurality of building block arrays.

In an embodiment, the present invention includes a composition including a support with a portion of the support including a plurality of building blocks. The building blocks are coupled to the support. The composition can include or be an artificial receptor, a heterogeneous building block array, or a composition including a surface and a region on the surface.

In an embodiment, the present invention includes an artificial receptor including a plurality of building blocks coupled to a support.

In an embodiment, the present invention includes a heterogeneous building block array. This array includes a support and a plurality of spots on the support. The spots include a plurality of building blocks. The building blocks are coupled to the support.

In an embodiment, the present invention includes a composition including a surface and a region on the surface. This region includes a plurality of building blocks, the building blocks being coupled to the support.

In an embodiment, the present invention includes a composition of matter including a plurality of building blocks.

In an embodiment, the building blocks include framework, linker, first recognition element, and second recognition element or have a formula linker-framework-(first recognition element)(second recognition element). The framework can be an amino acid. The building block can have the formula: 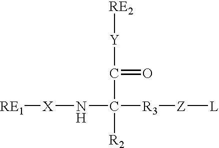
in which: X, Y, Z, R₂, R₃, RE₁, RE₂ and L are described hereinbelow.

### BRIEF DESCRIPTION OF THE FIGURES

FIG. 1 schematically illustrates two dimensional representations of an embodiment of a receptor according to the present invention that employs 4 different building blocks to make a ligand binding site.

FIG. 2 schematically illustrates two and three dimensional representations of an embodiment of a molecular configuration of 4 building blocks, each building block including a recognition element, a framework, and a linker coupled to a support (immobilization/anchor).

FIG. 3 schematically illustrates binding space divided qualitatively into 4 quadrants—large hydrophilic, large hydrophobic, small hydrophilic, and small lipophilic.

FIG. 4 illustrates a plot of volume versus logP for 81 building blocks including each of the 9 A and 9 B recognition elements.

FIGS. 5A and 5B illustrate a plot of volume versus logP for combinations of building blocks with A and B recognition elements forming candidate artificial receptors.

FIG. 5B represents a detail from FIG. 5A. This detail illustrates that the candidate artificial receptors fill the binding space evenly.

FIG. 6 illustrates that candidate artificial receptors made up of building blocks can be sorted and evaluated with respect to their nearest neighbors, other candidate artificial receptors made up of one or more of the same building blocks.

FIG. 7A schematically illustrates representative structures of the support floor and building blocks according to the present invention on a surface of a support.

FIG. 7B schematically illustrates a support coupled to a signal element, a building block, and a modified floor element.

FIG. 8 schematically illustrates representative space filing structures of a candidate artificial receptor according to the present invention including both an amine floor and a four building block receptor.

FIG. 9 schematically illustrates a glass support including pendant amine or amide structures.

FIG. 10 schematically illustrates employing successive subsets of the available building blocks to develop a lead or working artificial receptor.

FIG. 11 schematically illustrates identification of a lead artificial receptor from among candidate artificial receptors.

FIG. 12 schematically illustrates a false color fluorescence image of a labeled microarray according to an embodiment of the present invention.

FIG. 13 schematically illustrates a two dimensional plot of data obtained for candidate artificial receptors contacted with and/or binding phycoerythrin.

FIG. 14 schematically illustrates a three dimensional plot of data obtained for candidate artificial receptors contacted with and/or binding phycoerythrin.

FIG. 15 schematically illustrates a two dimensional plot of data obtained for candidate artificial receptors contacted with and/or binding a fluorescent derivative of ovalbumin.

FIG. 16 schematically illustrates a three dimensional plot of data obtained for candidate artificial receptors contacted with and/or binding a fluorescent derivative of ovalbumin.

FIG. 17 schematically illustrates a two dimensional plot of data obtained for candidate artificial receptors contacted with and/or binding a fluorescent derivative of bovine serum albumin.

FIG. 18 schematically illustrates a three dimensional plot of data obtained for candidate artificial receptors contacted with and/or binding a fluorescent derivative of bovine serum albumin.

FIG. 19 schematically illustrates a two dimensional plot of data obtained for candidate artificial receptors contacted with and/or binding an acetylated horseradish peroxidase.

FIG. 20 schematically illustrates a three dimensional plot of data obtained for candidate artificial receptors contacted with and/or binding an acetylated horseradish peroxidase.

FIG. 21 schematically illustrates a two dimensional plot of data obtained for candidate artificial receptors contacted with and/or binding a TCDD derivative of horseradish peroxidase.

FIG. 22 schematically illustrates a three dimensional plot of data obtained for candidate artificial receptors contacted with and/or binding a TCDD derivative of horseradish peroxidase.

FIG. 23 schematically illustrates a subset of the data illustrated in FIG. 14.

FIG. 24 schematically illustrates a subset of the data illustrated in FIG. 14.

FIG. 25 schematically illustrates a subset of the data illustrated in FIG. 14.

FIG. 26 schematically illustrates a correlation of binding data for phycoerythrin against logP for the building blocks making up the artificial receptor.

FIG. 27 schematically illustrates a correlation of binding data for phycoerythrin against logP for the building blocks making up the artificial receptor.

FIG. 28 schematically illustrates a two dimensional plot comparing data obtained for candidate artificial receptors contacted with and/or binding phycoerythrin to data obtained for candidate artificial receptors contacted with and/or binding a fluorescent derivative of bovine serum albumin.

FIGS. 29, 30, and 31 schematically illustrate subsets of data from FIGS. 14, 18, and 16, respectively, and demonstrate that the array of artificial receptors according to the present invention yields receptors distinguished between three analytes, phycoerythrin, bovine serum albumin, and ovalbumin.

FIG. 32 schematically illustrates a gray scale image of the fluorescence signal from a scan of a control plate which was prepared by washing off the building blocks with organic solvent before incubation with the test ligand.

FIG. 33 schematically illustrates a gray scale image of the fluorescence signal from a scan of an experimental plate which was incubated with 1.0 μg/ml Cholera Toxin B at 23° C.

FIG. 34 schematically illustrates a gray scale image of the fluorescence signal from a scan of an experimental plate which was incubated with 1.0 μg/ml Cholera Toxin. B at 3° C.

FIG. 35 schematically illustrates a gray scale image of the fluorescence signal from a scan of an experimental plate which was incubated with 1.0 μg/ml Cholera Toxin B at 43° C.

FIGS. 36-38 schematically illustrate plots of the fluorescence signals obtained from the candidate artificial receptors illustrated in FIGS. 33-35.

FIG. 39 schematically illustrate plots of the fluorescence signals obtained from the combinations of building blocks employed in the present studies, when those building blocks are covalently linked to the support. Binding was conducted at 23° C.

FIG. 40 schematically illustrates a graph of the changes in fluorescence signal from individual combinations of building blocks at 4° C., 23° C., or 44° C.

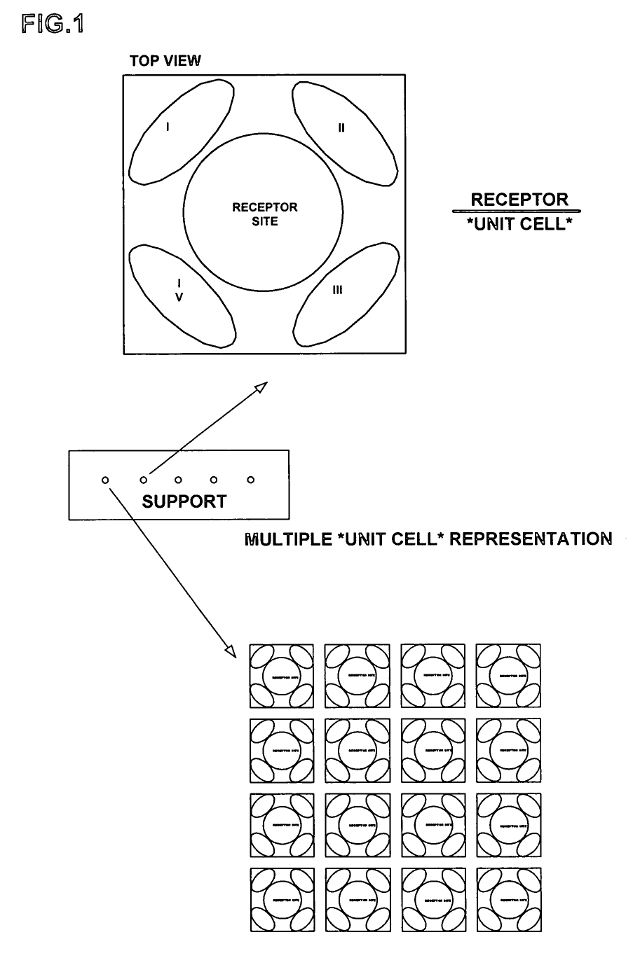

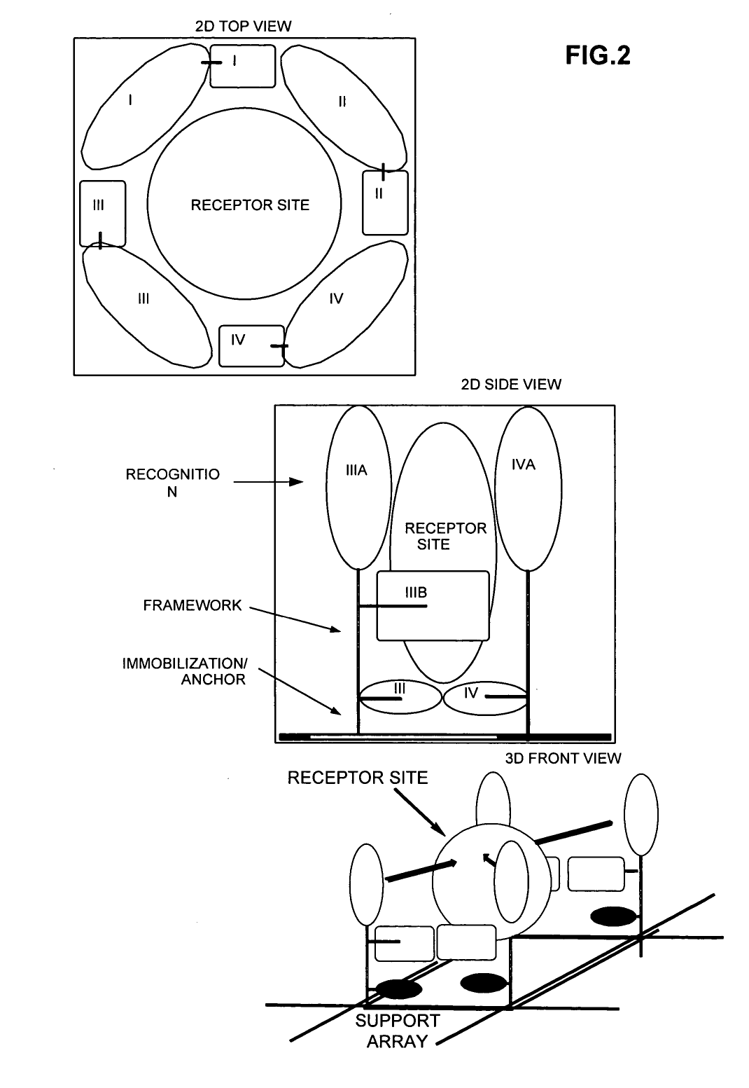

### DETAILED DESCRIPTION

Definitions

A combination of building blocks immobilized on, for example, a support can be a candidate artificial receptor, a lead artificial receptor, or a working artificial receptor. That is, a heterogeneous building block spot on a slide or a plurality of building blocks coated on a tube or well can be a candidate artificial receptor, a lead artificial receptor, or a working artificial receptor. A candidate artificial receptor can become a lead artificial receptor, which can become a working artificial receptor.

As used herein the phrase “candidate artificial receptor” refers to an immobilized combination of building blocks that can be tested to determine whether or not a particular test ligand binds to that combination. In an embodiment, the candidate artificial receptor can be a heterogeneous building block spot on a slide or a plurality of building blocks coated on a tube or well.

As used herein the phrase “lead artificial receptor” refers to an immobilized combination of building blocks that binds a test ligand at a predetermined concentration of test ligand, for example at 10, 1, 0.1, or 0.01 μg/ml, or at 1, 0.1, or 0.01 ng/ml. In an embodiment, the lead artificial receptor can be a heterogeneous building block spot on a slide or a plurality of building blocks coated on a tube or well.

As used herein the phrase “working artificial receptor” refers to a combination of building blocks that binds a test ligand with a selectivity and/or sensitivity effective for categorizing or identifying the test ligand. That is, binding to that combination of building blocks describes the test ligand as belonging to a category of test ligands or as being a particular test ligand. A working artificial receptor can, for example, bind the ligand at a concentration of, for example, 100, 10, 1, 0.1, 0.01, or 0.001 ng/ml. In an embodiment, the working artificial receptor can be a heterogeneous building block spot on a slide or a plurality of building blocks coated on a tube, well, slide, or other support or on a scaffold.

As used herein the phrase “working artificial receptor complex” refers to a plurality of artificial receptors, each a combination of building blocks, that binds a test ligand with a pattern of selectivity and/or sensitivity effective for categorizing or identifying the test ligand. That is, binding to the several receptors of the complex describes the test ligand as belonging to a category of test ligands or as being a particular test ligand. The individual receptors in the complex can each bind the ligand at different concentrations or with different affinities. For example, the individual receptors in the complex can each bind the ligand at concentrations of 100, 10, 1, 0.1, 0.01 or 0.001 ng/ml. In an embodiment, the working artificial receptor complex can be a plurality of heterogeneous building block spots or regions on a slide; a plurality of wells, each coated with a different combination of building blocks; or a plurality of tubes, each coated with a different combination of building blocks.

As used herein, the term “building block” refers to a molecular component of an artificial receptor including portions that can be envisioned as or that include one or more linkers, one or more frameworks, and one or more recognition elements. In an embodiment, the building block includes a linker, a framework, and one or more recognition elements. The building block interacts with the ligand.

As used herein, the term “linker” refers to a portion of or functional group on a building block that can be employed to or that does couple the building block to a support, for example, through a covalent link (e.g., a readily reversible covalent bond), ionic interaction, electrostatic interaction, or hydrophobic interaction.

As used herein, the term “framework” refers to a portion of a building block including the linker or to which the linker is coupled and to which one or more recognition elements are coupled.

As used herein, the term “recognition element” refers to a portion of a building block coupled to the framework but not covalently coupled to the support. Although not limiting to the present invention, the recognition element can provide or form one or more groups, surfaces, or spaces for interacting with the ligand.

As used herein, the phrase “plurality of building blocks” refers to two or more building blocks of different structure in a mixture, in a kit, or on a support or scaffold. Each building block has a particular structure, and use of building blocks in the plural, or of a plurality of building blocks, refers to more than one of these particular structures. Building blocks or plurality of building blocks does not refer to a plurality of molecules each having the same structure.

As used herein, the phrase “combination of building blocks” refers to a plurality of building blocks that together are in a spot, region, or a candidate, lead, or working artificial receptor. A combination of building blocks can be a subset of a set of building blocks. For example, a combination of building blocks can be one of the possible combinations of 2, 3, 4, 5, or 6 building blocks from a set of N (e.g., N=10-200) building blocks.

As used herein, the phrases “homogenous immobilized building block” and “homogenous immobilized building blocks” refer to a support or spot having immobilized on or within it only a single building block.

As used herein, the phrase “activated building block” refers to a building block activated to make it ready to form a covalent bond to a functional group, for example, on a support. A building block including a carboxyl group can be converted to a building block including an activated ester group, which is an activated building block. An activated building block including an activated ester group can react, for example, with an amine to form a covalent bond.

As used herein, the term “naïve” used with respect to one or more building blocks refers to a building block that has not previously been determined or known to bind to a test ligand of interest. For example, the recognition element(s) on a naïve building block has not previously been determined or known to bind to a test ligand of interest. A building block that is or includes a known ligand (e.g., GM1) for a particular protein (test ligand) of interest (e.g., cholera toxin) is not naïve with respect to that protein (test ligand).

As used herein, the term “immobilized” used with respect to building blocks coupled to a support refers to building blocks being stably oriented on the support so that they do not migrate on the support. Building blocks can be immobilized by covalent coupling, by ionic interactions, by electrostatic interactions, such as ion pairing, or by hydrophobic interactions, such as van der Waals interactions.

As used herein a “region” of a support, tube, well, or surface refers to a contiguous portion of the support, tube, well, or surface. Building blocks coupled to a region can refer to building blocks in proximity to one another in that region.

As used herein, a “bulky” group on a molecule is larger than a moiety including 7 or 8 carbon atoms.

As used herein, a “small” group on a molecule is hydrogen, methyl, or another group smaller than a moiety including 4 carbon atoms.

As used herein, the term “lawn” refers to a layer, spot, or region of functional groups on a support, which can be at a density sufficient to place coupled building blocks in proximity to one another. The functional groups can include groups capable of forming covalent, ionic, electrostatic, or hydrophobic interactions with building blocks.

As used herein, the term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₁₂ for straight chain, C₁-C₆ for branched chain). Likewise, cycloalkyls can have from 3-10 carbon atoms in their ring structure or can have 5, 6 or 7 carbons in the ring structure.

The term “alkyl” as used herein refers to both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aryl alkyl, or an aromatic or heteroaromatic moiety. The moieties substituted on the hydrocarbon chain can themselves be substituted, if appropriate. For example, the substituents of a substituted alkyl can include substituted and unsubstituted forms of the groups listed above.

The phrase “aryl alkyl”, as used herein, refers to an alkyl group substituted with an aryl group (e.g., an aromatic or heteroaromatic group).

As used herein, the terms “alkenyl” and “alkynyl” refer to unsaturated aliphatic groups analogous in length and optional substitution to the alkyls groups described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents such as those described above for alkyl groups. The term “aryl” also includes polycyclic ring systems having two or more cyclic rings in which two or more carbons are common to two adjoining rings (the rings are “fused rings”) wherein at least one of the rings is aromatic, e.g., the other cyclic ring(s) can be cycloalkyls, cycloalkenyls, cycloalkynyls, aryls and/or heterocyclyls.

As used herein, the terms “heterocycle” or “heterocyclic group” refer to 3- to 12-membered ring structures, for example 3- to 7-membered rings, whose ring structures include one to four heteroatoms. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents such as those described for alkyl groups.

As used herein, the term “heteroatom” as used herein means an atom of any element other than carbon or hydrogen, such as nitrogen, oxygen, sulfur and phosphorous.

Artificial Receptors with Immobilized Building Blocks

Methods of Making Artificial Receptors

The present invention relates to a method of making an artificial receptor or a candidate artificial receptor. In an embodiment, this method includes preparing a spot or region on a support, the spot or region including a plurality of building blocks immobilized on the support. The method can include forming a plurality of spots on a solid support, each spot including a plurality of building blocks, and immobilizing (e.g., reversibly) a plurality of building blocks on the solid support in each spot. In an embodiment, an array of such spots is referred to as a heterogeneous building block array.

The method can include mixing a plurality of building blocks and employing the mixture in forming the spot(s). Alternatively, the method can include spotting individual building blocks on the support. Coupling building blocks to the support can employ covalent bonding or noncovalent interactions. Suitable noncovalent interactions include interactions between ions, hydrogen bonding, van der Waals interactions, and the like. In an embodiment, the support can be functionalized with moieties that can engage in covalent bonding or noncovalent interactions. Forming spots can yield a microarray of spots of heterogeneous combinations of building blocks, each of which can be a candidate artificial receptor. The method can apply or spot building blocks onto a support in combinations of 2, 3, 4, or more building blocks.

In an embodiment, the present method can be employed to produce a solid support having on its surface a plurality of regions or spots, each region or spot including a plurality of building blocks. For example, the method can include spotting a glass slide with a plurality of spots, each spot including a plurality of building blocks. Such a spot can be referred to as including heterogeneous building blocks. A plurality of spots of building blocks can be referred to as an array of spots.

In an embodiment, the present method includes making a receptor surface. Making a receptor surface can include forming a region on a solid support, the region including a plurality of building blocks, and immobilizing (e.g., reversibly) the plurality of building blocks to the solid support in the region. The method can include mixing a plurality of building blocks and employing the mixture in forming the region or regions. Alternatively, the method can include applying individual building blocks in a region on the support. Forming a region on a support can be accomplished, for example, by soaking a portion of the support with the building block solution. The resulting coating including building blocks can be referred to as including heterogeneous building blocks.

A region including a plurality of building blocks can be independent and distinct from other regions including a plurality of building blocks. In an embodiment, one or more regions including a plurality of building blocks can overlap to produce a region including the combined pluralities of building blocks. In an embodiment, two or more regions including a single building block can overlap to form one or more regions each including a plurality of building blocks. The overlapping regions can be envisioned, for example, as portions of overlap in a Ven diagram, or as portions of overlap in a pattern like a plaid or tweed.

In an embodiment, the method produces a spot or surface with a density of building blocks sufficient to provide interactions of more than one building block with a ligand. That is, the building blocks can be in proximity to one another. Proximity of different building blocks can be detected by determining different (e.g., greater) binding of a test ligand to a spot or surface including a plurality of building blocks compared to a spot or surface including only one of the building blocks.

In an embodiment, the method includes forming an array of heterogeneous spots made from combinations of a subset of the total building blocks and/or smaller groups of the building blocks in each spot. That is, the method forms spots including only, for example, 2 or 3 building blocks, rather than 4 or 5. For example, the method can form spots from combinations of a full set of building blocks (e.g. 81 of a set of 81) in groups of 2 and/or 3. For example, the method can form spots from combinations of a subset of the building blocks (e.g., 25 of the set of 81) in groups of 4 or 5. For example, the method can form spots from combinations of a subset of the building blocks (e.g., 25 of a set of 81) in groups of 2 or 3. The method can include forming additional arrays incorporating building blocks, lead artificial receptors, or structurally similar building blocks.

In an embodiment, the method includes forming an array including one or more spots that function as controls for validating or evaluating binding to artificial receptors of the present invention. In an embodiment, the method includes forming one or more regions, tubes, or wells that function as controls for validating or evaluating binding to artificial receptors of the present invention. Such a control spot, region, tube, or well can include no building block, only a single building block, only functionalized lawn, or combinations thereof.

The method can immobilize (e.g., reversibly) building blocks on supports using known methods for immobilizing compounds of the types employed as building blocks. Coupling building blocks to the support can employ covalent bonding or noncovalent interactions. Suitable noncovalent interactions include interactions between ions, hydrogen bonding, van der Waals interactions, and the like. In an embodiment, the support can be functionalized with moieties that can engage in reversible covalent bonding, moieties that can engage in noncovalent interactions, a mixture of these moieties, or the like.

In an embodiment, the support can be functionalized with moieties that can engage in covalent bonding, e.g., reversible covalent bonding. The present invention can employ any of a variety of the numerous known functional groups, reagents, and reactions for forming reversible covalent bonds. Suitable reagents for forming reversible covalent bonds include those described in Green, T W; Wuts, P G M (1999), Protective Groups in Organic Synthesis Third Edition, Wiley-Interscience, New York, 779 pp. For example, the support can include functional groups such as a carbonyl group, a carboxyl group, a silane group, boric acid or ester, an amine group (e.g., a primary, secondary, or tertiary amine, a hydroxylamine, a hydrazine, or the like), a thiol group, an alcohol group (e.g., primary, secondary, or tertiary alcohol), a diol group (e.g., a 1,2 diol or a 1,3 diol), a phenol group, a catechol group, or the like. These functional groups can form groups with reversible covalent bonds, such as ether (e.g., alkyl ether, silyl ether, thioether, or the like), ester (e.g., alkyl ester, phenol ester, cyclic ester, thioester, or the like), acetal (e.g., cyclic acetal), ketal (e.g., cyclic ketal), silyl derivative (e.g., silyl ether), boronate (e.g., cyclic boronate), amide, hydrazide, imine, carbamate, or the like. Such a functional group can be referred to as a covalent bonding moiety, e.g., a first covalent bonding moiety.

A carbonyl group on the support and an amine group on a building block can form an imine or Schiff's base. The same is true of an amine group on the support and a carbonyl group on a building block. A carbonyl group on the support and an alcohol group on a building block can form an acetal or ketal. The same is true of an alcohol group on the support and a carbonyl group on a building block. A thiol (e.g., a first thiol) on the support and a thiol (e.g., a second thiol) on the building block can form a disulfide.

A carboxyl group on the support and an alcohol group on a building block can form an ester. The same is true of an alcohol group on the support and a carboxyl group on a building block. Any of a variety of alcohols and carboxylic acids can form esters that provide covalent bonding that can be reversed in the context of the present invention. For example, reversible ester linkages can be formed from alcohols such as phenols with electron withdrawing groups on the aryl ring, other alcohols with electron withdrawing groups acting on the hydroxyl-bearing carbon, other alcohols, or the like; and/or carboxyl groups such as those with electron withdrawing groups acting on the acyl carbon (e.g., nitrobenzylic acid, R—CF₂—COOH, R—CCl₂—COOH, and the like), other carboxylic acids, or the like.

In an embodiment, the support, matrix, or lawn can be functionalized with moieties that can engage in noncovalent interactions. For example, the support can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like.

In an embodiment, the support, matrix, or lawn includes a charged moiety (e.g., a first charged moiety). Suitable charged moieties include positively charged moieties and negatively charged moieties. Suitable positively charged moieties (e.g., at neutral pH in aqueous compositions) include amines, quaternary ammonium moieties, ferrocene, or the like. Suitable negatively charged moieties (e.g., at neutral pH in aqueous compositions) include carboxylates, phenols substituted with strongly electron withdrawing groups (e.g., tetrachlorophenols), phosphates, phosphonates, phosphinates, sulphates, sulphonates, thiocarboxylates, hydroxamic acids, or the like.

In an embodiment, the support, matrix, or lawn includes groups that can hydrogen bond (e.g., a first hydrogen bonding group), either as donors or acceptors. The support, matrix, or lawn can include a surface or region with groups that can hydrogen bond. For example, the support, matrix, or lawn can include a surface or region including one or more carboxyl groups, amine groups, hydroxyl groups, carbonyl groups, or the like. Ionic groups can also participate in hydrogen bonding.

In an embodiment, the support, matrix, or lawn includes a lipophilic moiety (e.g., a first lipophilic moiety). Suitable lipophilic moieties include branched or straight chain C₆₋₃₆ alkyl, C₈₋₂₄ alkyl, C₁₂₋₂₄ alkyl, C₁₂₋₁₈ alkyl, or the like; C₆₋₃₆alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. A lipophilic moiety like a quaternary ammonium lipophilic moiety can also include a positive charge.

Artificial Receptors

A candidate artificial receptor, a lead artificial receptor, or a working artificial receptor includes combination of building blocks immobilized (e.g., reversibly) on, for example, a support. An individual artificial receptor can be a heterogeneous building block spot on a slide or a plurality of building blocks coated on a slide, tube, or well. The building blocks can be immobilized through any of a variety of interactions, such as covalent, electrostatic, or hydrophobic interactions. For example, the building block and support or lawn can each include one or more functional groups or moieties that can form covalent, electrostatic, hydrogen bonding, van der Waals, or like interactions.

An array of candidate artificial receptors can be a commercial product sold to parties interested in using the candidate artificial receptors as implements in developing receptors for test ligands of interest. In an embodiment, a useful array of candidate artificial receptors includes at least one glass slide, the at least one glass slide including spots of a predetermined number of combinations of members of a set of building blocks, each combination including a predetermined number of building blocks.

One or more lead artificial receptors can be developed from a plurality of candidate artificial receptors. In an embodiment, a lead artificial receptor includes a combination of building blocks and binds detectable quantities of test ligand upon exposure to, for example, several picomoles of test ligand at a concentration of 1, 0.1, or 0.01 μg/ml, or at 1, 0.1, or 0.01 ng/ml test ligand; at a concentration of 0.01 μg/ml, or at 1, 0.1, or 0.01 ng/ml test ligand; or a concentration of 1, 0.1, or 0.01 ng/ml test ligand.

Artificial receptors, particularly candidate or lead artificial receptors, can be in the form of an array of artificial receptors. Such an array can include, for example, 1.66 million spots, each spot including one combination of 4 building blocks from a set of 81 building blocks. Such an array can include, for example, 28,000 spots, each spot including one combination of 2 or 3 building blocks from a set of 19 building blocks. Each spot is a candidate artificial receptor and a combination of building blocks. The array can also be constructed to include lead artificial receptors. For example, the array of artificial receptors can include combinations of fewer building blocks and/or a subset of the building blocks.

In an embodiment, an array of candidate artificial receptors includes building blocks of general Formula 2 (shown hereinbelow), with RE₁ being B1, B2, B3, B3a, B4, B5, B6, B7, B8, or B9 (shown hereinbelow) and with RE₂ being A1, A2, A3, A3a, A4, A5, A6, A7, A8, or A9 (shown hereinbelow). In an embodiment, the framework is tyrosine.

One or more working artificial receptors can be developed from one or more lead artificial receptors. In an embodiment, a working artificial receptor includes a combination of building blocks and binds categorizing or identifying quantities of test ligand upon exposure to, for example, several picomoles of test ligand at a concentration of 100, 10, 1, 0.1, 0.01, or 0.001 ng/ml test ligand; at a concentration of 10, 1, 0.1, 0.01, or 0.001 ng/ml test ligand; or a concentration of 1, 0.1, 0.01, or 0.001 ng/ml test ligand.

In an embodiment, the artificial receptor of the invention includes a plurality of building blocks coupled to a support. In an embodiment, the plurality of building blocks can include or be building blocks of Formula 2 (shown below). An abbreviation for the building block including a linker, a tyrosine framework, and recognition elements AxBy is TyrAxBy. In an embodiment, a candidate artificial receptor can include combinations of building blocks of formula TyrA1B1, TyrA2B2, TyrA2B4, TyrA2B6, TyrA2B8, TyrA3B3, TyrA4B2, TyrA4B4, TyrA4B6, TyrA4B8, TyrA5B5, TyrA6B2, TyrA6B4, TyrA6B6, TyrA6B8, TyrA7B7, TyrA8B2, TyrA8B4, TyrA8B6, or TyrA8B8.

Building Blocks

The present invention relates to building blocks for making or forming candidate artificial receptors. Building blocks can be designed, made, and selected to provide a variety of structural characteristics among a small number of compounds. A building block can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A building block can be bulky or it can be small.

A building block can be visualized as including several components, such as one or more frameworks, one or more linkers, and/or one or more recognition elements. The framework can be covalently coupled to each of the other building block components. The linker can be covalently coupled to the framework. The linker can be coupled to a support through one or more of covalent, electrostatic, hydrogen bonding, van der Waals, or like interactions. The recognition element can be covalently coupled to the framework. In an embodiment, a building block includes a framework, a linker, and a recognition element. In an embodiment, a building block includes a framework, a linker, and two recognition elements.

A description of general and specific features and functions of a variety of building blocks and their synthesis can be found in copending U.S. patent application Ser. No. 10/244,727, filed Sep. 16, 2002, and Application No. PCT/US03/05328, filed Feb. 19, 2003, each entitled “ARTIFICIAL RECEPTORS, BUILDING BLOCKS, AND METHODS”, and U.S. Provisional Patent Application Ser. No. ______, also entitled “ARTIFICIAL RECEPTORS, BUILDING BLOCKS, AND METHODS”, filed ______, the disclosures of which are incorporated herein by reference. These patent documents include, in particular, a detailed written description of: function, structure, and configuration of building blocks, framework moieties, recognition elements, synthesis of building blocks, specific embodiments of building blocks, specific embodiments of recognition elements, and sets of building blocks.

Framework

The framework can be selected for functional groups that provide for coupling to the recognition moiety and for coupling to or being the linking moiety. The framework can interact with the ligand as part of the artificial receptor. In an embodiment, the framework includes multiple reaction sites with orthogonal and reliable functional groups and with controlled stereochemistry. Suitable functional groups with orthogonal and reliable chemistries include, for example, carboxyl, amine, hydroxyl, phenol, carbonyl, and thiol groups, which can be individually protected, deprotected, and derivatized. In an embodiment, the framework has two, three, or four functional groups with orthogonal and reliable chemistries. In an embodiment, the framework has three functional groups. In such an embodiment, the three functional groups can be independently selected, for example, from carboxyl, amine, hydroxyl, phenol, carbonyl, or thiol group. The framework can include alkyl, substituted alkyl, cycloalkyl, heterocyclic, substituted heterocyclic, aryl alkyl, aryl, heteroaryl, heteroaryl alkyl, and like moieties.

A general structure for a framework with three functional groups can be represented by Formula 1a: 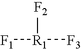
A general structure for a framework with four functional groups can be represented by Formula 1b: 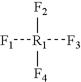
In these general structures: R₁ can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group; and F₁, F₂, F₃, or F₄ can independently be a carboxyl, amine, hydroxyl, phenol, carbonyl, or thiol group. F₁, F₂, F₃, or F₄ can independently be a 1-12, a 1-6, a 1-4 carbon alkyl, substituted alkyl, cycloalkyl, heterocyclic, substituted heterocyclic, aryl alkyl, aryl, heteroaryl, heteroaryl alkyl, or inorganic group substituted with carboxyl, amine, hydroxyl, phenol, carbonyl, or thiol group. F₃ and/or F₄ can be absent.

A variety of compounds fit the formulas and text describing the framework including amino acids, and naturally occurring or synthetic compounds including, for example, oxygen and sulfur functional groups. The compounds can be racemic, optically active, or achiral. For example, the compounds can be natural or synthetic amino acids, α-hydroxy acids, thioic acids, and the like.

Suitable molecules for use as a framework include a natural or synthetic amino acid, particularly an amino acid with a functional group (e.g., third functional group) on its side chain. Amino acids include carboxyl and amine functional groups. The side chain functional group can include, for natural amino acids, an amine (e.g., alkyl amine, heteroaryl amine), hydroxyl, phenol, carboxyl, thiol, thioether, or amidino group. Natural amino acids suitable for use as frameworks include, for example, serine, threonine, tyrosine, aspartic acid, glutamic acid, asparagine, glutamine, cysteine, lysine, arginine, histidine. Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties as framework and with carboxyl, amine, hydroxyl, phenol, carbonyl, or thiol functional groups. Suitable synthetic amino acids include β-amino acids and homo or β analogs of natural amino acids. In an embodiment, the framework amino acid can be serine, threonine, or tyrosine, e.g., serine or tyrosine, e.g., tyrosine.

Although not limiting to the present invention, a framework amino acid, such as serine, threonine, or tyrosine, with a linker and two recognition elements can be visualized with one of the recognition elements in a pendant orientation and the other in an equatorial orientation, relative to the extended carbon chain of the framework.

All of the naturally occurring and many synthetic amino acids are commercially available. Further, forms of these amino acids derivatized or protected to be suitable for reactions for coupling to recognition element(s) and/or linkers can be purchased or made by known methods (see, e.g., Green, T W; Wuts, P G M (1999), Protective Groups in Organic Synthesis Third Edition, Wiley-Interscience, New York, 779 pp.; Bodanszky, M.; Bodanszky, A. (1994), The Practice of Peptide Synthesis Second Edition, Springer-Verlag, New York, 217 pp.).

Recognition Element

The recognition element can be selected to provide one or more structural characteristics to the building block. The framework can interact with the ligand as part of the artificial receptor. For example, the recognition element can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A recognition element can be a small group or it can be bulky.

In an embodiment the recognition element can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group. The recognition element can be substituted with a group that includes or imparts positive charge, negative charge, acid, base, electron acceptor, electron donor, hydrogen bond donor, hydrogen bond acceptor, free electron pair, π electrons, charge polarization, hydrophilicity, hydrophobicity, and the like.

Recognition elements with a positive charge (e.g., at neutral pH in aqueous compositions) include amines, quaternary ammonium moieties, sulfonium, phosphonium, ferrocene, and the like. Suitable amines include alkyl amines, alkyl diamines, heteroalkyl amines, aryl amines, heteroaryl amines, aryl alkyl amines, pyridines, heterocyclic amines (saturated or unsaturated, the nitrogen in the ring or not), amidines, hydrazines, and the like. Alkyl amines generally have 1 to 12 carbons, e.g., 1-8, and rings can have 3-12 carbons, e.g., 3-8. Suitable alkyl amines include that of formula B9. Suitable heterocyclic or alkyl heterocyclic amines include that of formula A9. Suitable pyridines include those of formulas A5 and B5. Any of the amines can be employed as a quaternary ammonium compound. Additional suitable quaternary ammonium moieties include trimethyl alkyl quaternary ammonium moieties, dimethyl ethyl alkyl quaternary ammonium moieties, dimethyl alkyl quaternary ammonium moieties, aryl alkyl quaternary ammonium moieties, pyridinium quaternary ammonium moieties, and the like.

Recognition elements with a negative charge (e.g., at neutral pH in aqueous compositions) include carboxylates, phenols substituted with strongly electron withdrawing groups (e.g., substituted tetrachlorophenols), phosphates, phosphonates, phosphinates, sulphates, sulphonates, thiocarboxylates, and hydroxamic acids. Suitable carboxylates include alkyl carboxylates, aryl carboxylates, and aryl alkyl carboxylates. Suitable phosphates include phosphate mono-, di-, and tri-esters, and phosphate mono-, di-, and tri-amides. Suitable phosphonates include phosphonate mono- and di-esters, and phosphonate mono- and di-amides (e.g., phosphonamides). Suitable phosphinates include phosphinate esters and amides.

Recognition elements with a negative charge and a positive charge (at neutral pH in aqueous compositions) include sulfoxides, betaines, and amine oxides.

Acidic recognition elements can include carboxylates, phosphates, sulphates, and phenols. Suitable acidic carboxylates include thiocarboxylates. Suitable acidic phosphates include the phosphates listed hereinabove.

Basic recognition elements include amines. Suitable basic amines include alkyl amines, aryl amines, aryl alkyl amines, pyridines, heterocyclic amines (saturated or unsaturated, the nitrogen in the ring or not), amidines, and any additional amines listed hereinabove. Suitable alkyl amines include that of formula B9. Suitable heterocyclic or alkyl heterocyclic amines include that of formula A9. Suitable pyridines include those of formulas A5 and B5.

Recognition elements including a hydrogen bond donor include amines, amides, carboxyls, protonated phosphates, protonated phosphonates, protonated phosphinates, protonated sulphates, protonated sulphinates, alcohols, and thiols. Suitable amines include alkyl amines, aryl amines, aryl alkyl amines, pyridines, heterocyclic amines (saturated or unsaturated, the nitrogen in the ring or not), amidines, ureas, and any other amines listed hereinabove. Suitable alkyl amines include that of formula B9. Suitable heterocyclic or alkyl heterocyclic amines include that of formula A9. Suitable pyridines include those of formulas A5 and B5. Suitable protonated carboxylates, protonated phosphates include those listed hereinabove. Suitable amides include those of formulas A8 and B8. Suitable alcohols include primary alcohols, secondary alcohols, tertiary alcohols, and aromatic alcohols (e.g., phenols). Suitable alcohols include those of formulas A7 (a primary alcohol) and B7 (a secondary alcohol).

Recognition elements including a hydrogen bond acceptor or one or more free electron pairs include amines, amides, carboxylates, carboxyl groups, phosphates, phosphonates, phosphinates, sulphates, sulphonates, alcohols, ethers, thiols, and thioethers. Suitable amines include alkyl amines, aryl amines, aryl alkyl amines, pyridines, heterocyclic amines (saturated or unsaturated, the nitrogen in the ring or not), amidines, ureas, and amines as listed hereinabove. Suitable alkyl amines include that of formula B9. Suitable heterocyclic or alkyl heterocyclic amines include that of formula A9. Suitable pyridines include those of formulas A5 and B5. Suitable carboxylates include those listed hereinabove. Suitable amides include those of formulas A8 and B8. Suitable phosphates, phosphonates and phosphinates include those listed hereinabove. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include alkyl ethers, aryl alkyl ethers. Suitable alkyl ethers include that of formula A6. Suitable aryl alkyl ethers include that of formula A4. Suitable thioethers include that of formula B6.

Recognition elements including uncharged polar or hydrophilic groups include amides, alcohols, ethers, thiols, thioethers, esters, thioesters, boranes, borates, and metal complexes. Suitable amides include those of formulas A8 and B8. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include those listed hereinabove. Suitable ethers include that of formula A6. Suitable aryl alkyl ethers include that of formula A4.

Recognition elements including uncharged hydrophobic groups include alkyl (substituted and unsubstituted), alkene (conjugated and unconjugated), alkyne (conjugated and unconjugated), aromatic. Suitable alkyl groups include lower alkyl, substituted alkyl, cycloalkyl, aryl alkyl, and heteroaryl alkyl. Suitable lower alkyl groups include those of formulas A1, A3, A3a, and B1. Suitable aryl alkyl groups include those of formulas A3, A3a, A4, B3, B3a, and B4. Suitable alkyl cycloalkyl groups include that of formula B2. Suitable alkene groups include lower alkene and aryl alkene. Suitable aryl alkene groups include that of formula B4. Suitable aromatic groups include unsubstituted aryl, heteroaryl, substituted aryl, aryl alkyl, heteroaryl alkyl, alkyl substituted aryl, and polyaromatic hydrocarbons. Suitable aryl alkyl groups include those of formulas A3, A3a and B4. Suitable alkyl heteroaryl groups include those of formulas A5 and B5.

Spacer (e.g., small) recognition elements include hydrogen, methyl, ethyl, and the like. Bulky recognition elements include 7 or more carbon or hetero atoms.

Formulas A1-A9 and B1-B9 are:

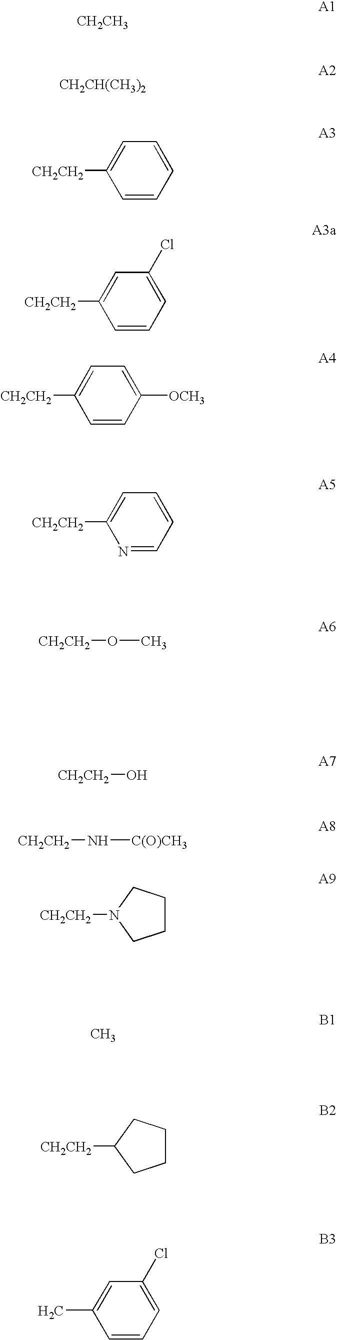

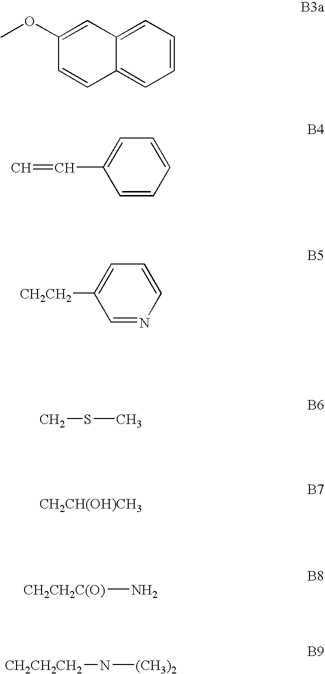

These A and B recognition elements can be called derivatives of, according to a standard reference: A1, ethylamine; A2, isobutylamine; A3, phenethylamine; A4, 4-methoxyphenethylamine; A5,2-(2-aminoethyl)pyridine; A6,2-methoxyethylamine; A7, ethanolamine; A8, N-acetylethylenediamine; A9,1-(2-aminoethyl)pyrrolidine; B1, acetic acid, B2, cyclopentylpropionic acid; B3,3-chlorophenylacetic acid; B4, cinnamic acid; B5, 3-pyridinepropionic acid; B6, (methylthio)acetic acid; B7,3-hydroxybutyric acid; B8, succinamic acid; and B9,4-(dimethylamino)butyric acid.

In an embodiment, the recognition elements include one or more of the structures represented by formulas A1, A2, A3, A3a, A4, A5, A6, A7, A8, and/or A9 (the A recognition elements) and/or B1, B2, B3, B3a, B4, B5, B6, B7, B8, and/or B9 (the B recognition elements). In an embodiment, each building block includes an A recognition element and a B recognition element. In an embodiment, a group of 81 such building blocks includes each of the 81 unique combinations of an A recognition element and a B recognition element. In an embodiment, the A recognition elements are linked to a framework at a pendant position. In an embodiment, the B recognition elements are linked to a framework at an equatorial position. In an embodiment, the A recognition elements are linked to a framework at a pendant position and the B recognition elements are linked to the framework at an equatorial position.

Although not limiting to the present invention, it is believed that the A and B recognition elements represent the assortment of functional groups and geometric configurations employed by polypeptide receptors. Although not limiting to the present invention, it is believed that the A recognition elements represent six advantageous functional groups or configurations and that the addition of functional groups to several of the aryl groups increases the range of possible binding interactions. Although not limiting to the present invention, it is believed that the B recognition elements represent six advantageous functional groups, but in different configurations than employed for the A recognition elements. Although not limiting to the present invention, it is further believed that this increases the range of binding interactions and further extends the range of functional groups and configurations that is explored by molecular configurations of the building blocks.

In an embodiment, the building blocks including the A and B recognition elements can be visualized as occupying a binding space defined by lipophilicity/hydrophilicity and volume. A volume can be calculated (using known methods) for each building block including the various A and B recognition elements. A measure of lipophilicity/hydrophilicity (logP) can be calculated (using known methods) for each building block including the various A and B recognition elements. Negative values of logP show affinity for water over nonpolar organic solvent and indicate a hydrophilic nature. A plot of volume versus logP can then show the distribution of the building blocks through a binding space defined by size and lipophilicity/hydrophilicity.

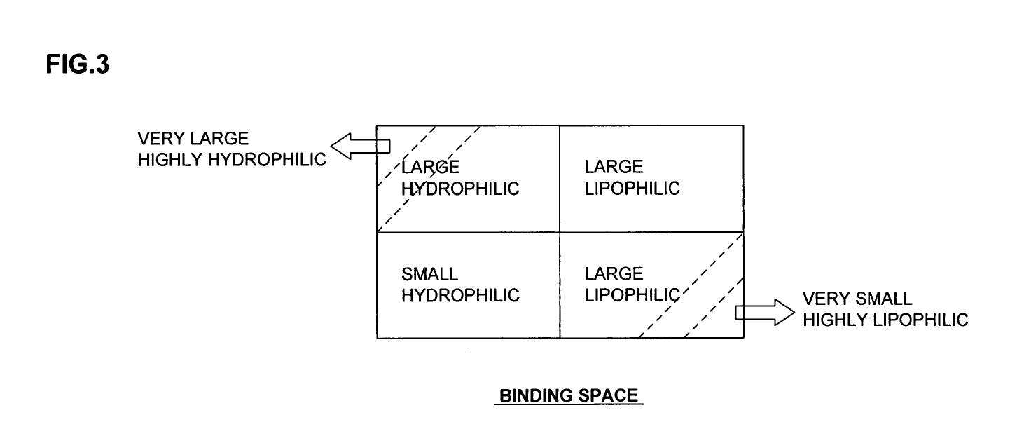

FIG. 3 schematically illustrates binding space divided qualitatively into 4 quadrants—large hydrophilic, large hydrophobic, small hydrophilic, and small lipophilic. FIG. 3 denotes a small triangle of the large hydrophilic quadrant as very large and highly hydrophilic. FIG. 3 denotes a small triangle of the small lipophilic quadrant as very small and highly lipophilic.

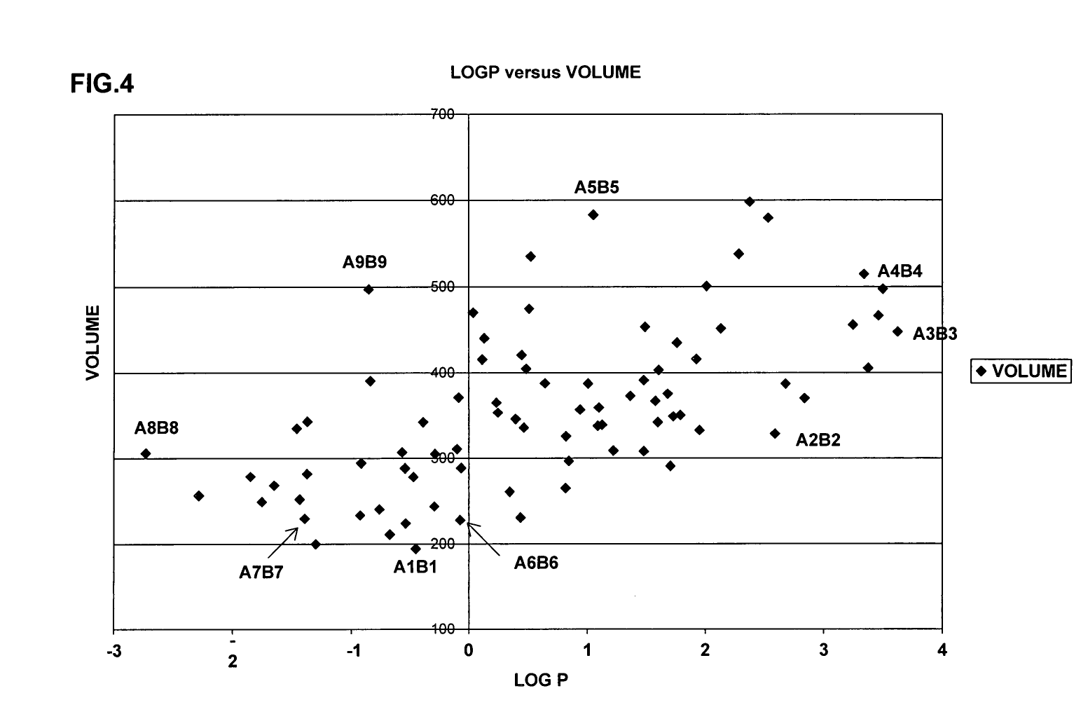

FIG. 4 illustrates a plot of volume versus logP for 81 building blocks including each of the 9 A and 9 B recognition elements. This plot illustrates that the 81 building blocks with A and B recognition elements fill a significant portion of the binding space defined by volume and lipophilicity/hydrophilicity. The space filled by the 81 building blocks is roughly bounded by the A1B1, A2B2, . . . A9B9 building blocks (FIG. 4). The 81 building blocks with A and B recognition elements fill a majority of this binding space excluding only the portion denoted very large and highly hydrophilic and the portion denoted very small and highly lipophilic.

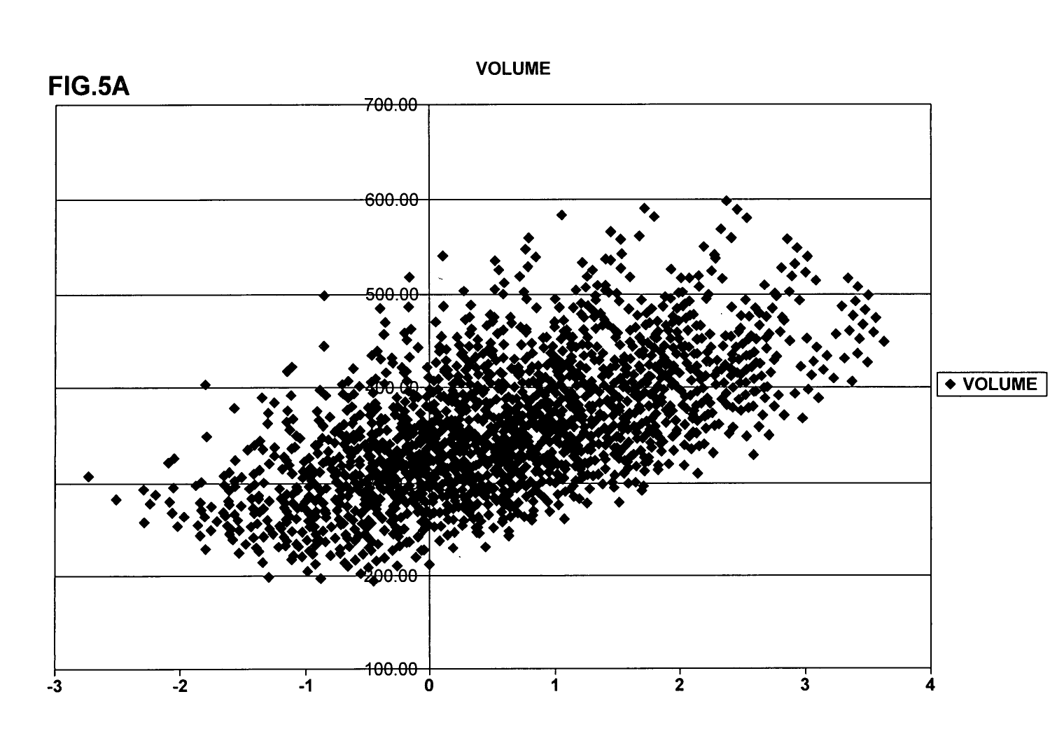

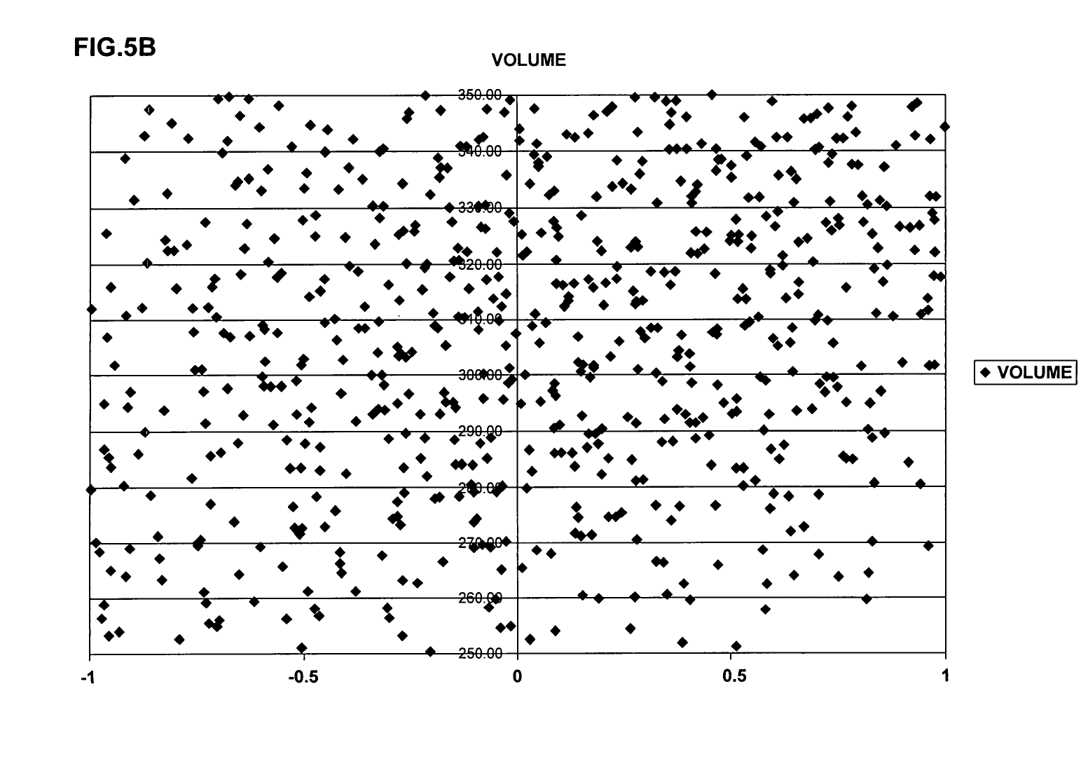

FIGS. 5A and 5B illustrate a plot of volume versus logP for combinations of building blocks with A and B recognition elements forming candidate artificial receptors. The volumes and values of logP for these candidate artificial receptors generally fill in the space occupied by the individual building blocks. FIG. 5B represents a detail from FIG. 5A. This detail illustrates that the candidate artificial receptors fill the binding space evenly.

Candidate artificial receptors made from building blocks with A and B recognition elements include receptors with a wide range of sizes and a wide range of values of lipophilicity/hydrophilicity.

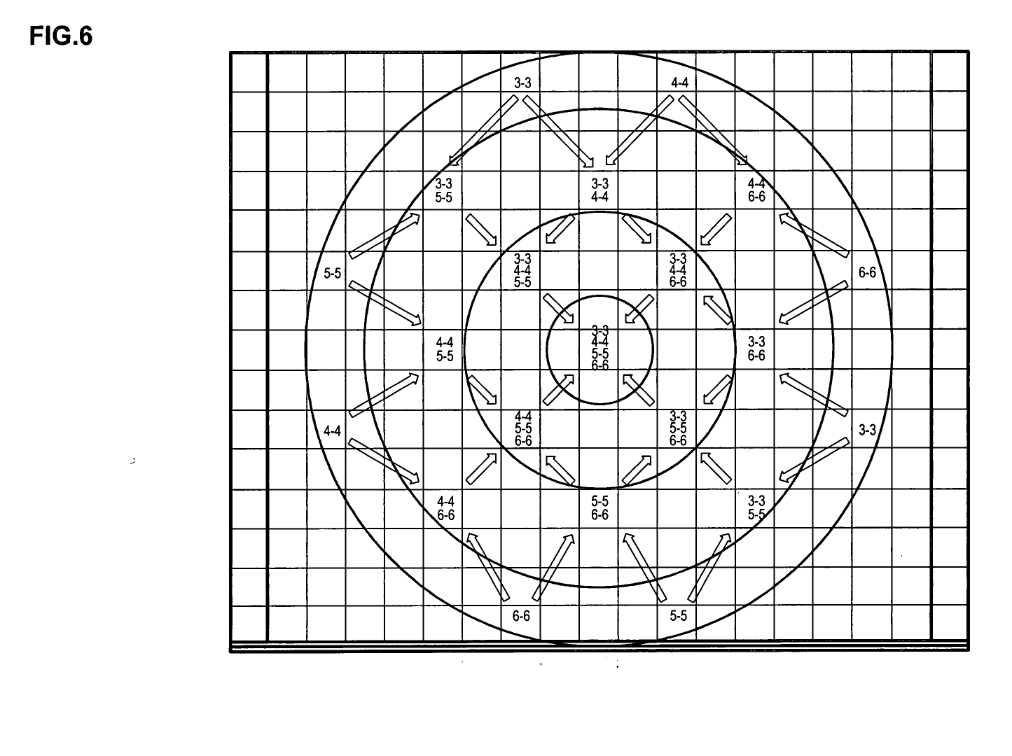

FIG. 6 illustrates that candidate artificial receptors made up of building blocks can be sorted and evaluated with respect to their nearest neighbors, other candidate artificial receptors made up of one or more of the same building blocks. In an embodiment, the nearest neighbor can be made up of a subset of the building blocks forming the subject candidate artificial receptor. For example, as shown in FIG. 6, a candidate artificial receptor made up of TyrA3B3/TyrA4B4/TyrA5B5/TyrA6B6 has among its nearest neighbors candidate artificial receptors TyrA4B4/TyrA5B5/TyrA6B6, TyrA3B3/TyrA5B5/TyrA6B6, TyrA3B3/TyrA4B4/TyrA6B6, and TyrA3B3/TyrA4B4/TyrA5B5. These candidate artificial receptors in turn have additional nearest neighbors. Candidate receptors and/or recognition elements can also be grouped as neighbors based on lipophilicity/hydrophilicity, size, charge, or another physical or chemical characteristic.

Reagents that form many of the recognition elements are commercially available. For example, reagents for forming recognition elements A1, A2, A3, A3a, A4, A5, A6, A7, A8, A9 B1, B2, B3, B3a, B4, B5, B6, B7, B8, and B9 are commercially available.

Linkers

The linker is selected to provide a suitable covalent attachment of the building block to a support. The framework can interact with the ligand as part of the artificial receptor. The linker can also provide bulk, distance from the support, hydrophobicity, hydrophilicity, and like structural characteristics to the building block. In an embodiment, the linker forms a covalent bond with a functional group on the framework.

In an embodiment, before attachment to the support the linker also includes a functional group that can be activated to react with or that will react with a functional group on the support. In such an embodiment, the linker can form or can be visualized as forming a covalent bond with an alcohol, phenol, thiol, amine, carbonyl, or like group on the framework. The linker can include a carboxyl, alcohol, phenol, thiol, amine, carbonyl, maleimide, or like group that can react with or be activated to react with the support. Between the bond to the framework and the group formed by the attachment to the support, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

Coupling building blocks to the support can employ covalent bonding or noncovalent interactions. Suitable noncovalent interactions include interactions between ions, hydrogen bonding, van der Waals interactions, and the like. In an embodiment, the linker includes moieties that can engage in covalent bonding or noncovalent interactions. In an embodiment, the linker includes moieties that can engage in covalent bonding. Suitable groups for forming covalent and reversible covalent bonds are described hereinabove.

In an embodiment, the linker includes moieties that can engage in noncovalent interactions. For example, the linker can include functional groups such as an ionic group, a group that can hydrogen bond, or a group that can engage in van der Waals or other hydrophobic interactions. Such functional groups can include cationic groups, anionic groups, lipophilic groups, surface active groups, and the like. In an embodiment, the linker includes a charged moiety (e.g., a first charged moiety). Suitable charged moieties include positively charged moieties and negatively charged moieties. Suitable positively charged moieties are described hereinabove. Suitable negatively charged moieties are described hereinabove. In an embodiment, the linker includes a lipophilic moiety (e.g., a first lipophilic moiety). Suitable lipophilic moieties are described hereinabove.

For example, suitable linkers can include: the functional group participating in or formed by the bond to the framework, the functional group or groups participating in or formed by the reversible interaction with the support or lawn, and a linker backbone moiety. The linker backbone moiety can include about 4 to about 48 carbon or heteroatoms, about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms, or the like. The linker backbone can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, mixtures thereof, or like moiety.

In an embodiment, the linker includes a lipophilic moiety, the functional group participating in or formed by the bond to the framework, and, optionally, one or more moieties for forming a reversible covalent bond, a hydrogen bond, or an ionic interaction. In such an embodiment, the lipophilic moiety can have about 4 to about 48 carbons, about 8 to about 14 carbons, about 12 to about 24 carbons, about 16 to about 18 carbons, or the like. In such an embodiment, the linker can include about 1 to about 8 reversible bond/interaction moieties or about 2 to about 4 reversible bond/interaction moieties. Suitable linkers have structures such as (CH₂)ₙCOOH, with n=12-24, n=17-24, or n=16-18.

Suitable linker groups include those of formula: (CH₂)ₙCOOH, with n=1-16, n=2-8, n=2-6, or n=3. Reagents that form suitable linkers are commercially available and include any of a variety of reagents with orthogonal functionality.

Embodiments of Building Blocks

In an embodiment, building blocks can be represented by Formula 2: 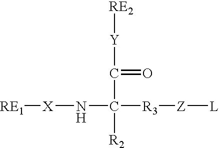
in which: RE₁ is recognition element 1, RE₂ is recognition element 2, and L is a linker. X is absent, C═O, CH₂, NR, NR₂, NH, NHCONH, SCONH, CH═N, or OCH₂NH. In certain embodiments, X is absent or C═O. Y is absent, NH, O, CH₂, or NRCO. In certain embodiments, Y is NH or O. In an embodiment, Y is NH. Z is CH₂, O, NH, S, CO, NR, NR₂, NHCONH, SCONH, CH═N, or OCH₂NH. In an embodiment, Z is O. R₂ is H, CH₃, or another group that confers chirality on the building block and has size similar to or smaller than a methyl group. R₃ is CH₂; CH₂-phenyl; CHCH₃; (CH₂)ₙ with n=2-3; or cyclic alkyl with 3-8 carbons, e.g., 5-6 carbons, phenyl, naphthyl. In certain embodiments, R₃ is CH₂ or CH₂-phenyl.

RE₁ is B1, B2, B3, B3a, B4, B5, B6, B7, B8, B9, A1, A2, A3, A3a, A4, A5, A6, A7, A8, or A9. In certain embodiments, RE₁ is B1, B2, B3, B3a, B4, B5, B6, B7, B8, or B9. RE₂ is A1, A2, A3, A3a, A4, A5, A6, A7, A8, A9, B1, B2, B3, B3a, B4, B5, B6, B7, B8, or B9. In certain embodiments, RE₂ is A1, A2, A3, A3a, A4, A5, A6, A7, A8, or A9. In an embodiment, RE₁ can be B2, B3a, B4, B5, B6, B7, or B8. In an embodiment, RE₂ can be A2, A3a, A4, A5, A6, A7, or A8.

In an embodiment, L is the functional group participating in or formed by the bond to the framework (such groups are described herein), the functional group or groups participating in or formed by the reversible interaction with the support or lawn (such groups are described herein), and a linker backbone moiety. In an embodiment, the linker backbone moiety is about 4 to about 48 carbon or heteroatom alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or mixtures thereof; or about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms.

In an embodiment, the L is the functional group participating in or formed by the bond to the framework (such groups are described herein) and a lipophilic moiety (such groups are described herein) of about 4 to about 48 carbons, about 8 to about 14 carbons, about 12 to about 24 carbons, about 16 to about 18 carbons. In an embodiment, this L also includes about 1 to about 8 reversible bond/interaction moieties (such groups are described herein) or about 2 to about 4 reversible bond/interaction moieties. In an embodiment, L is (CH₂)ₙCOOH, with n=12-24, n=17-24, or n=16-18.

In an embodiment, L is (CH₂)ₙCOOH, with n=1-16, n=2-8, n=4-6, or n=3.

Building blocks including an A and/or a B recognition element, a linker, and an amino acid framework can be made by methods illustrated in general Scheme 1. 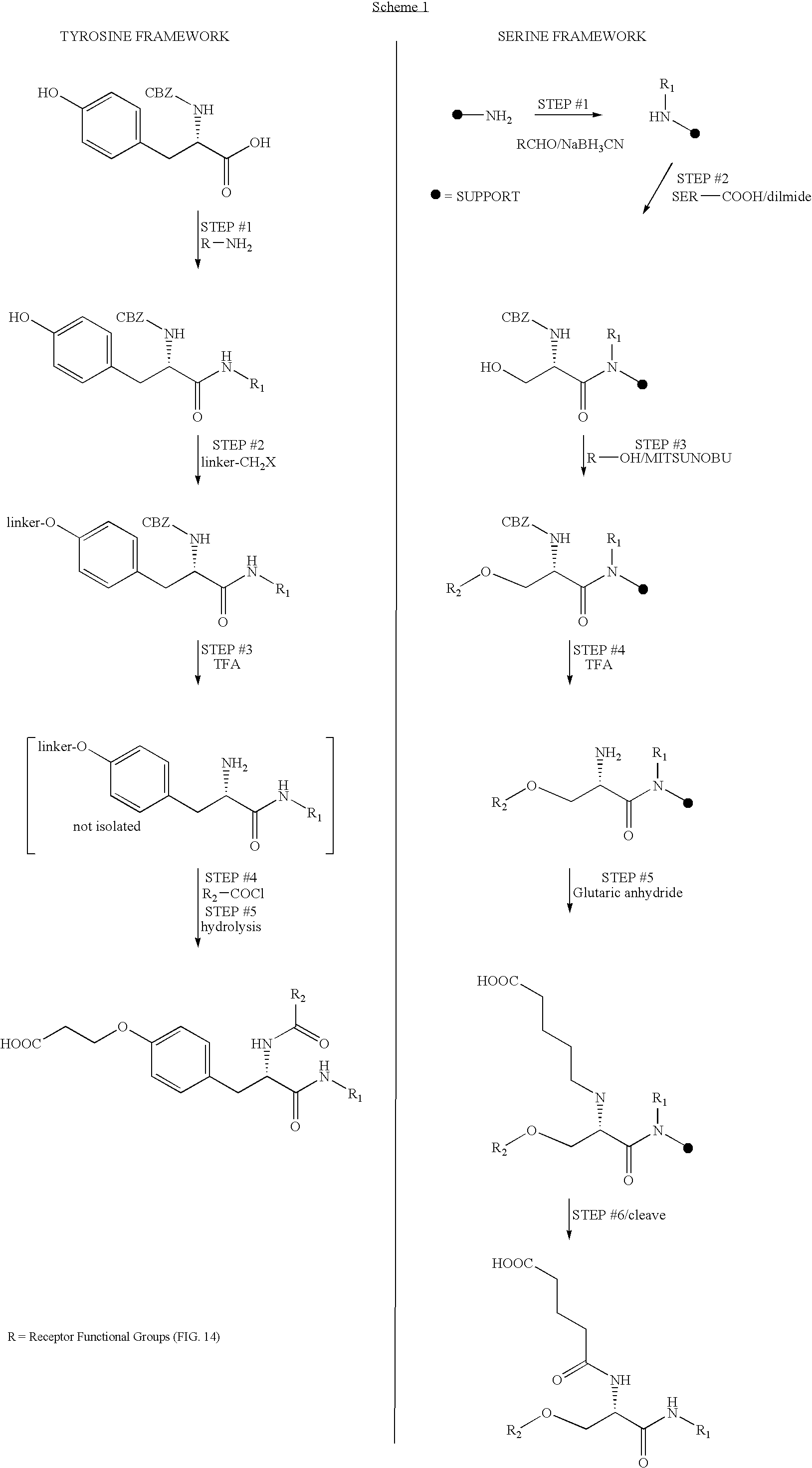
More on Building Blocks

Building blocks can be asymmetric. Employing asymmetry, various combinations of, for example, linker and recognition elements can produce building blocks that can be visualized to occupy 3D space in different ways. As a consequence, these different building blocks can perform binding related but otherwise distinct functions.

In an embodiment, building blocks including two recognition elements, a linker, and a framework can be visualized as having both recognition elements in spreading pendant configurations. Such a configuration has a molecular footprint with substantial area in two dimensions. Such a larger footprint can be suitable, for example, for binding larger ligands that prefer or require interactions with a receptor over a larger area or that prefer or require interactions with a larger number of functional groups on the recognition element. Such larger ligands can include proteins, carbohydrates, cells, and microorganisms (e.g., bacteria and viruses).

In an embodiment, a building block can have only a single recognition element in a pendant configuration and a pendant linker distal on the framework. Such building blocks can be compact. Such a building block can interact with large molecules that include a binding region, such as a protein (e.g., enzyme or receptor) or other macromolecule. For example, such a building block can be employed to probe cavities, such as binding sites, on proteins.

Sets of Building Blocks

The present invention also relates to sets of building blocks. The sets of building blocks can include isolated building blocks, building blocks with an activated linker for coupling to a support, and/or building blocks coupled to a support. Sets of building blocks include a plurality of building blocks. The plurality of building blocks can be a component of a coating, of a spot or spots (e.g., forming candidate artificial receptor(s)), or of a kit. The plurality of building blocks can include a sufficient number of building blocks and recognition elements for exploring candidate artificial receptors or for defining receptors for a ligand. That is, the set of building blocks can include a majority (e.g., at least 6) of the structural characteristics selected from positive charge, negative charge, acid, base, electron acceptor, electron donor, hydrogen bond donor, hydrogen bond acceptor, free electron pair, π electrons, charge polarization, hydrophilicity, hydrophobicity.

For a set of building blocks, the recognition elements can be selected to provide a variety of structural characteristics to the individual members of the set. A single building block can include recognition elements with more than one of the structural characteristics. A set of building blocks can include recognition elements with each of the structural characteristics. For example, a set of building blocks can include one or more building blocks including a positively charged recognition element, one or more building blocks including a negatively charged recognition element, one or more building blocks including an acidic recognition element, one or more building blocks including a basic recognition element, one or more building blocks including an electron donating recognition element, one or more building blocks including an electron accepting recognition element, one or more building blocks including a hydrogen bond donor recognition element, one or more building blocks including a hydrogen bond acceptor recognition element, one or more building blocks including a polar recognition element, one or more building blocks including a recognition element with free electron pair(s), one or more building blocks including a recognition element with π electrons, one or more building blocks including a hydrophilic recognition element, one or more building blocks including a hydrophobic recognition element, one or more building blocks including a small recognition element, and/or one or more building blocks including a bulky recognition element.

In an embodiment, the number and variety of recognition elements is selected to provide a set of building blocks with a manageable number of members. A manageable number of building blocks provides, for example, fewer than 10 million combinations (e.g., about 2 million combinations), with each combination including, for example, 2, 3, 4, 5, or 6 building blocks. In an embodiment, the recognition elements provide a set of building blocks that incorporate the functional groups and configurations found in the components of natural receptors. This can advantageously be accomplished with a small set of building blocks. A set of building blocks can include building blocks of general Formula 2, with RE₁ being B1, B2, B3, B3a, B4, B5, B6, B7, B8, or B9 and with RE₂ being A1, A2, A3, A3a, A4, A5, A6, A7, A8, or A9.

FIGS. 4, 5A, and 5B illustrate plots of volume versus logP for building blocks including each of the 9 A and 9 B recognition elements and artificial receptors made from these building blocks. These plots illustrate that the building blocks with A and B recognition elements and artificial receptors made from these building blocks fill a significant portion of the binding space defined by volume and lipophilicity/hydrophilicity.

Embodiments of Sets of Building Blocks

The present invention includes sets of building blocks. Sets of building blocks can include 2 or more building blocks coupled to a support or scaffold. Such a support or scaffold can be referred to as including heterogeneous building blocks. As used herein, the term “support” refers to a solid support that is, for example, macroscopic. As used herein, the term scaffold refers to a molecular scale structure to which a plurality of building blocks can covalently bind. The two or more building blocks can be coupled to the support or scaffold in a molecular configuration with different building blocks in proximity to one another. Such a molecular configuration of a plurality of different building blocks provides a candidate artificial receptor. The present invention includes immobilized sets and combinations of building blocks. In an embodiment, the present invention includes a solid support having on its surface a plurality of building blocks.

Embodiments of Sets as Reagents

The present invention includes sets of building blocks as reagents. Reagent sets of building blocks can include individual or mixtures of building blocks. The reagent sets can be used to make immobilized building blocks and groups of building blocks, and can be sold for this purpose. In an embodiment, the set includes building blocks with recognition elements representing hydrophobic alkyl, hydrophobic aryl, hydrogen bond acceptor, basic, hydrogen bond donor, and small size as structural characteristics. For example, the set can include building blocks of general Formula 2, with RE₁ being B1, B2, B3, B3a, B4, B5, B6, B7, B8, or B9 and with RE₂ being A1, A2, A3, A3a, A4, A5, A6, A7, A8, or A9. The set can be part of a kit including containers of one or mixtures of building blocks, the containers can be in a package, and the kit can include written material describing the building blocks and providing instructions for their use.

Building Blocks and/or Lawns on Supports

Forming a spot on a support can be accomplished by methods and apparatus such as pin spotters (sometimes referred to as printers), which can, for example, spot 10,000 to more than 100,000 spots on a microscope slide. Other spotters include piezoelectric spotters (similar to ink jets) and electromagnetic spotters that can also spot, for example, 110,000 to more than 100,000 spots on a microscope slide. An array of spots can also be printed on the bottom of a well of a microtiter plate. Arrays can also be built using photolithography and other known processes that can produce spots containing building blocks on a substrate. In an embodiment, for spotting, the activated building blocks can be provided as mixtures made, for example, in large numbers in microwell plates by a robotic system.

Each spot in a microarray can include a statistically significant number of each building block. For example, although not limiting to the present invention, it is believed that each micro spot of a size sufficiently small that 100,000 fit on a microscope slide can include approximately 320 million combinations of 4 building blocks. Each spot can include a density of building blocks sufficient to provide interactions of more than one building block with a ligand. Such interactions can be determined as described above for regions. The method can include spotting the building blocks so that each spot is separated from the others.

In an embodiment, the method spots or the array includes building blocks in combinations of 2, 3, 4, or more. The method can form up to 100,000 or more spots on a glass slide. Therefore, for arrays, a manageable set of building blocks can provide several million combinations of building blocks. For example, in this context, a set of 81 building blocks provides a manageable number (1.66 million) of combinations of 4 building blocks. For convenience in limiting the number of slides employed or produced, in this embodiment a set includes up to 200 building blocks, e.g., 50-100 or about 80 (e.g., 81) building blocks.

For an embodiment employing a bulky tube or well, a manageable set of building blocks can provide fewer than several hundred or several thousand combinations of building blocks. For example, in this context, a set of 4, 5, or 6 building blocks provides a manageable number of combinations of 2, 3, or 4 building blocks. In an embodiment, the present invention can produce or include a plurality of tubes each tube having immobilized on its surface a heterogeneous combination of building blocks.

The method can apply or spot building blocks onto a support in combinations of 2 or 3 building blocks. Effective artificial receptors can be developed employing as few as several dozen or several hundred artificial receptors, that can include 2 and/or, preferably, 3 building blocks. Such artificial receptors can employ, for example, a tube, well, or slide as a support.

The method can employ building blocks including activated esters and couple them to supports including hydroxyl functional groups. The method can include activating a carboxyl group on a building block by derivatizing to form the activated ester. By Way of further example, the method can couple building blocks including hydroxyl functional groups to supports including carboxyl groups. Pairs of functional groups that can be employed on building blocks and supports according to the method include nucleophile/electrophile pairs, such as thiol and maleimide, alcohol and carboxyl (or activated carboxyl), mixtures thereof, and the like.

The support can include any functional group suitable for forming a covalent bond with a building block. The support or the building block can include a functional group such as alcohol, phenol, thiol, amine, carbonyl, or like group. The support or the building block can include a carboxyl, alcohol, phenol, thiol, amine, carbonyl, maleimide, or like group that can react with or be activated to react with the support or the building block. The support can include one or more of these groups. A plurality of building blocks can include a plurality of these groups.

The building blocks can be activated to react with a functional group on the support. Coupling can occur spontaneously after forming the spot of the building block or activated building block. The method can include mixing a plurality of activated building blocks and employing the mixture in forming the spot(s). Alternatively, the method can include spotting individual activated building blocks on the support.

The support or the building block (e.g., the linker) can include a good leaving group bonded to, for example, an alkyl or aryl group. The leaving group being “good” enough to be displaced by the alcohol, phenol, thiol, amine, carbonyl, or like group on the support or the building block. Such a support or the building block can include a moiety represented by the formula: R—X, in which X is a leaving group such as halogen (e.g., —Cl, —Br, or —I), tosylate, mesylate, triflate, and R is alkyl, substituted alkyl, cycloalkyl, heterocyclic, substituted heterocyclic, aryl alkyl, aryl, heteroaryl, or heteroaryl alkyl. The support can include one or more of these groups. A plurality of building blocks can include a plurality of these groups.

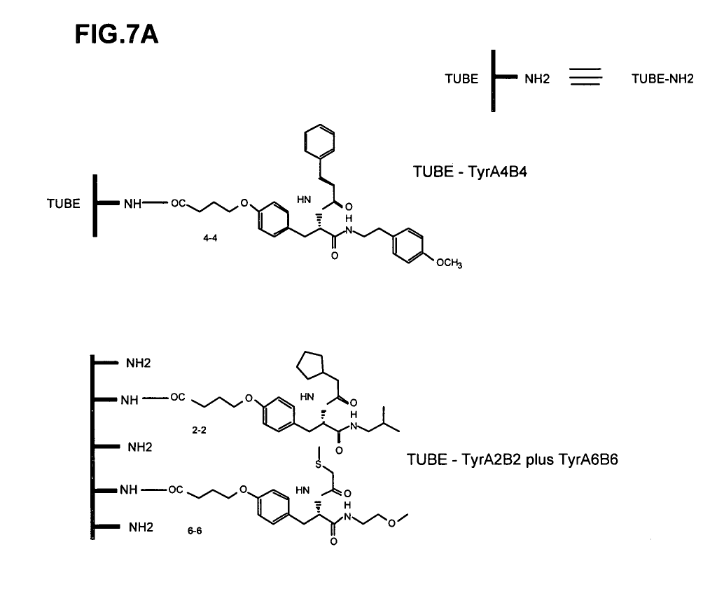

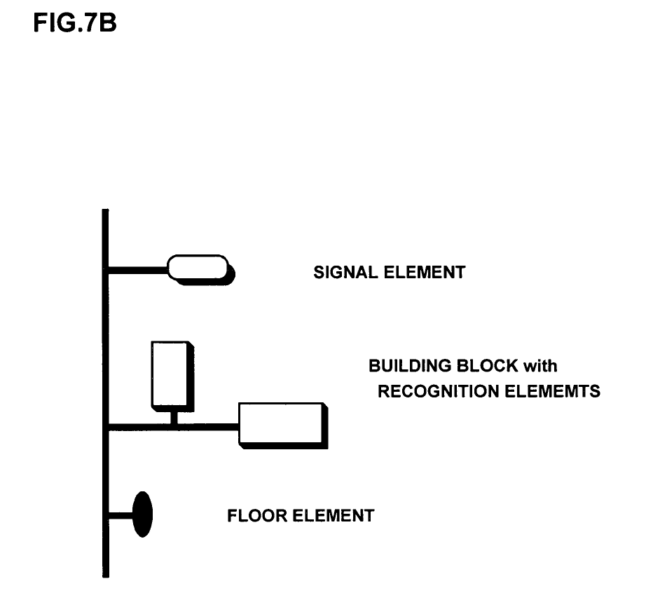

For example, a building block linker carboxyl group can be activated by reacting the building block with carbodiimide in the presence of sulfo N-hydroxysuccinimide in aqueous dimethylformamide. The activated building block can be reacted directly with an amine on a glass support (hereinafter amino glass). FIG. 7A illustrates that derivatization of only a portion of the amine groups on the support can be effective for producing candidate artificial receptors. Although not limiting to the present invention, it is believed that the amine load on the glass is in excess of that required for candidate artificial receptor preparation. Preparations of surfaces including combinations of building blocks can be accomplished by, for example, premixing of activated building blocks prior to addition to the amino tube or the sequential mixing of the coupling solutions in the tubes.

The method or article can employ any of the variety of known supports employed in combinatorial or synthetic chemistry (e.g., a microscope slide, a bead, a resin, a gel, or the like). Suitable supports include functionalized glass, such as a functionalized slide or tube, glass microscope slide, glass plate, glass coverslip, glass beads, microporous glass beads, microporous polymer beads (e.g. those sold under the tradename Stratospheres™), silica gel supports, and the like. Suitable supports with hydrophobic surfaces include micelles and reverse micelles. The support can include a support matrix of a compound or mixture of compounds having functional groups suitable for coupling to a building block. The support matrix can be, for example, a coating on a microscope slide or functionalizing groups on a bead, gel, or resin. Known support matrices are commercially available and/or include linkers with functional groups that are coupled beads, gels, or resins. The support matrix functional groups can be pendant from the support in groups of one (e.g., as a lawn of amines, a lawn of another functional group, or a lawn of a mixture of functional groups) or in groups of, for example, 2, 3, 4, 5, 6, or 7. The groups of a plurality of functional groups pendant from the support can be visualized as or can be scaffold molecules pendant from the support.

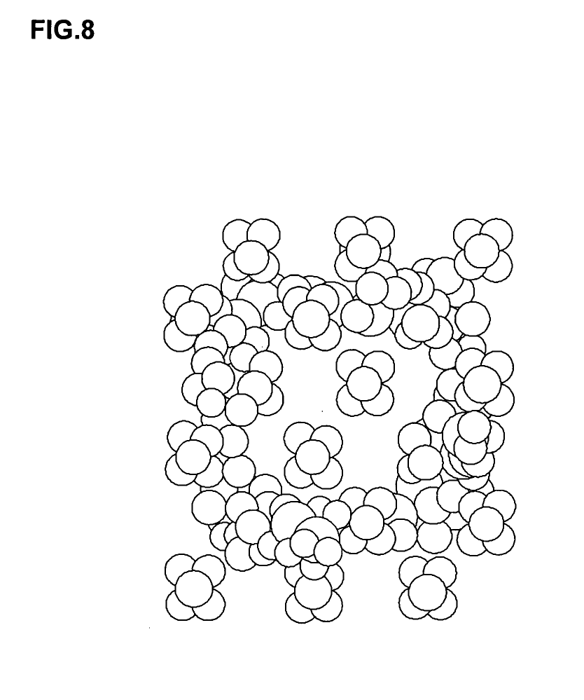

The surface of the support can be visualized as including a floor and the building blocks (FIGS. 7A, 7B, and 8). As illustrated in FIG. 7A, addition of building blocks to an amine lawn can proceed through reaction of the amines to form building block amides with some of the amines remaining on the floor of the support or candidate artificial receptor. Thus, the floor can be considered a feature of the candidate artificial receptor. The floor or modified floor can interact with the ligand as part of the artificial receptor. The nucleophilic or electrophilic groups on the floor can be left unreacted in the artificial receptor, or they can be modified. The floor can be modified with a small group that alters the recognition properties of the floor (FIG. 7B). The floor can be modified with a signal element that produces a detectable signal when a test ligand is bound to the receptor (FIG. 7B). For example, the signal element can be a fluorescent molecule that is quenched by binding to the artificial receptor. For example, the signal element can be a molecule that fluoresces only when binding occurs. The floor can be modified with a plurality of floor modifiers. For example, the floor can be modified with both a signal element and a small group that alters the recognition properties of the floor. One portion or region of the amine glass surface can be modified with a first floor modifier or lawn and another (e.g., second) portion or region can be modified with a second floor modifier or lawn.

In an embodiment, the candidate artificial receptor can include building blocks and unmodified amines of the floor. Such a candidate artificial receptor has an amine/ammonium floor. In an embodiment, the candidate artificial receptor can include building blocks and modified amines of the floor. For example, the floor amines can be modified by the simplest amide modification of the amines to form the acetamide (e.g., by reacting with acetic anhydride or acetyl chloride). Alternatively, the floor amines can be modified by reaction with succinic anhydride, benzoyl chloride, or the like.

A lawn or other coating of functional groups can be derivatized with a maximum density of building blocks by exposing the lawn to several equivalents of activated building blocks. For example, less than 1 (e.g., 0.1) or more (e.g., 10) equivalents can be sufficient for an adequate density of building blocks on the support to observe building-block-dependent binding of a ligand. An amine modified glass surface can be functionalized with building blocks, for example, by reaction with activated carboxyl derivatives to form an amide link to the lawn.

In an embodiment, a tube or well coated with a support matrix can be filled with activated building block (e.g., a solution containing activated building block), which couples to the support matrix. For example, the support can be a glass tube or well coated with a plurality of building blocks. The surface of the glass tube or well can be coated with a coating to which the plurality of building blocks become covalently bound.

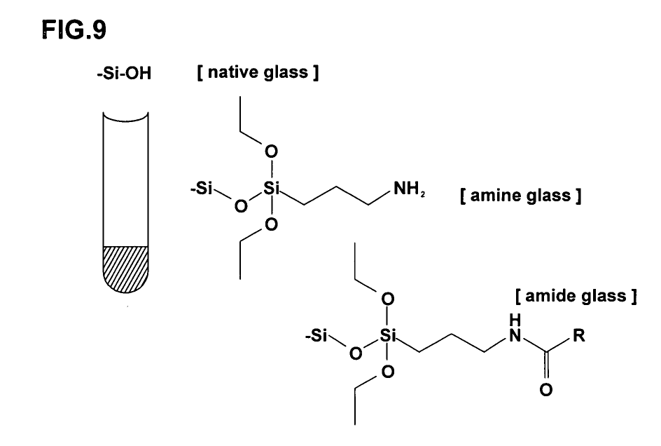

A commercially available glass support can be prepared for coupling building blocks by adding a support matrix to the surface of the support. The support matrix provides functional groups for coupling to the building block. Suitable support matrices include silanating agents. For example a glass tube (e.g., a 12×75 mm borosilicate glass tube from VWR) can be coated to form a lawn of amines by reaction of the glass with a silanating agent such as 3-aminopropyltriethoxysilane. Building blocks including an activated ester can be bound to this coating by reaction of the building block activated ester with the amine glass to form the amide bound building block. Starting with a commercially available slide, an amino functionalized slide from Corning, building blocks including an activated ester can be spotted on and covalently bound to the slide in a micro array by this same reaction. Such derivatization is schematically illustrated in FIG. 9.

In an embodiment, immobilized combinations of building blocks can include a plurality of tubes each tube having immobilized on its surface a heterogeneous combination of building blocks. The building blocks can be reversibly immobilized on the surface of the tube through covalent, electrostatic, hydrogen bonding, van der Waals, or like interactions. The immobilized building blocks can include combinations of 2, 3, or 4 building blocks. In an embodiment, the present invention includes a solid support having on its surface a plurality of regions or spots, each region or spot including a plurality of building blocks. For example, the support can be a glass slide spotted with a plurality of spots, each spot including a plurality of building blocks. A plurality of regions or spots of building blocks is referred to herein as an array of regions or spots.

In an embodiment, immobilized combinations of building blocks can include one or more glass slides, each slide having on its surface a plurality of spots, each spot including an immobilized heterogeneous combination of building blocks. The building blocks can be immobilized on the surface of the slide through covalent, electrostatic, hydrogen bonding, van der Waals, or like interactions. The immobilized building blocks can include, for example, combinations of 2, 3, 4, 5, or 6 building blocks.

In an embodiment, the one or more slides can include heterogeneous spots of building blocks made from combinations of a subset of the total building blocks and/or smaller groups of the building blocks in each spot. That is, each spot includes only, for example, 2 or 3 building blocks, rather than 4 or 5. For example, the one or more slides can include the number of spots formed by combinations of a full set of building blocks (e.g. 81 of a set of 81) in groups of 2 and/or 3. For example, the one or more slides can include the number of spots formed by combinations of a subset of the building blocks (e.g., 25 of the set of 81) in groups of 4 or 5. For example, the one or more slides can include the number of spots formed by combinations of a subset of the building blocks (e.g., 25 of the set of 81) in groups of 2 or 3. Should a candidate artificial receptor of interest be identified from the subset and/or smaller groups, then additional subsets and groups can be made or selected incorporating the building blocks in the candidates of interest or structurally similar building blocks.

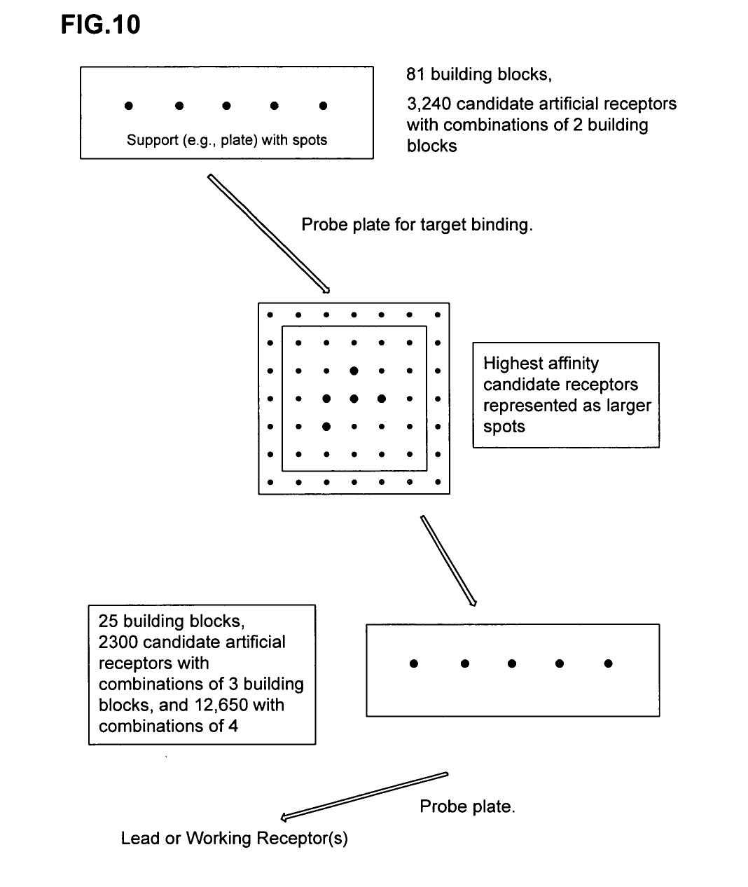

For example, FIG. 10 illustrates that a single slide with the 3,240 n=2 derived combinations can be used to define a more limited set from the 81 building blocks. This defined set of e.g. 25 (defined from a 5×5 matrix of the n=2 results) can be used to produce an additional 2,300 n=3 derived and 12,650 n=4 derived combinations which can be probed to define the optimum receptor configuration. Further optimization can be pursued using ratios of the best building blocks which deviate from 1:1 followed by specific synthesis of the identified receptor(s).

Using the Artificial Receptors

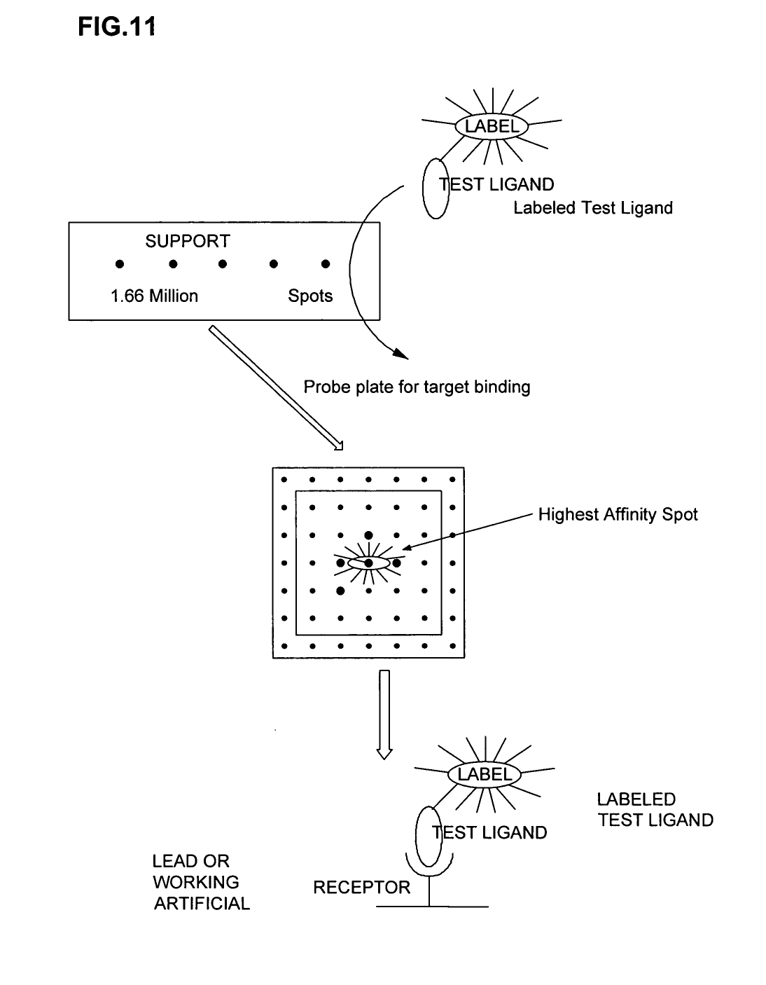

The present invention includes a method of using artificial receptors. The present invention includes a method of screening candidate artificial receptors to find lead artificial receptors that bind a particular test ligand. Detecting test ligand bound to a candidate artificial receptor can be accomplished using known methods for detecting binding to arrays on a slide or to coated tubes or wells. For example, the method can employ test ligand labeled with a detectable label, such as a fluorophore or an enzyme that produces a detectable product. Alternatively, the method can employ an antibody (or other binding agent) specific for the test ligand and including a detectable label. One or more of the spots that are labeled by the test ligand or that are more or most intensely labeled with the test ligand are selected as lead artificial receptors. The degree of labeling can be evaluated by evaluating the signal strength from the label. For example, the amount of signal can be directly proportional to the amount of label and binding. FIG. 11 provides a schematic illustration of an embodiment of this process.

Binding to an array of candidate receptors can be displayed in any of a variety of graphs, charts, or illustrations. For example, a two dimensional array of candidate receptors can be displayed with signal strength as a third dimension. Such a representation of the array can be illustrated as a bar graph with the height of the bar from each spot in the array representing the signal strength. This representation can be useful, for example, for locating those candidate receptors in an array that show signal strength well in excess of other candidate receptors.

Candidate receptors can also be displayed in a chart correlating binding signal strength with one or more properties of the receptor and/or its constituent building blocks. For example, each candidate receptor can be located on a graph of the volume of its building blocks versus its lipophilicity/hydrophilicity (see, e.g., FIGS. 3-5B). Again, signal strength can be illustrated as a third dimension. Those candidate receptors showing the greatest binding can then be found and evaluated with respect to candidate receptors with similar properties (e.g., volume and lipophilicity/hydrophilicity).

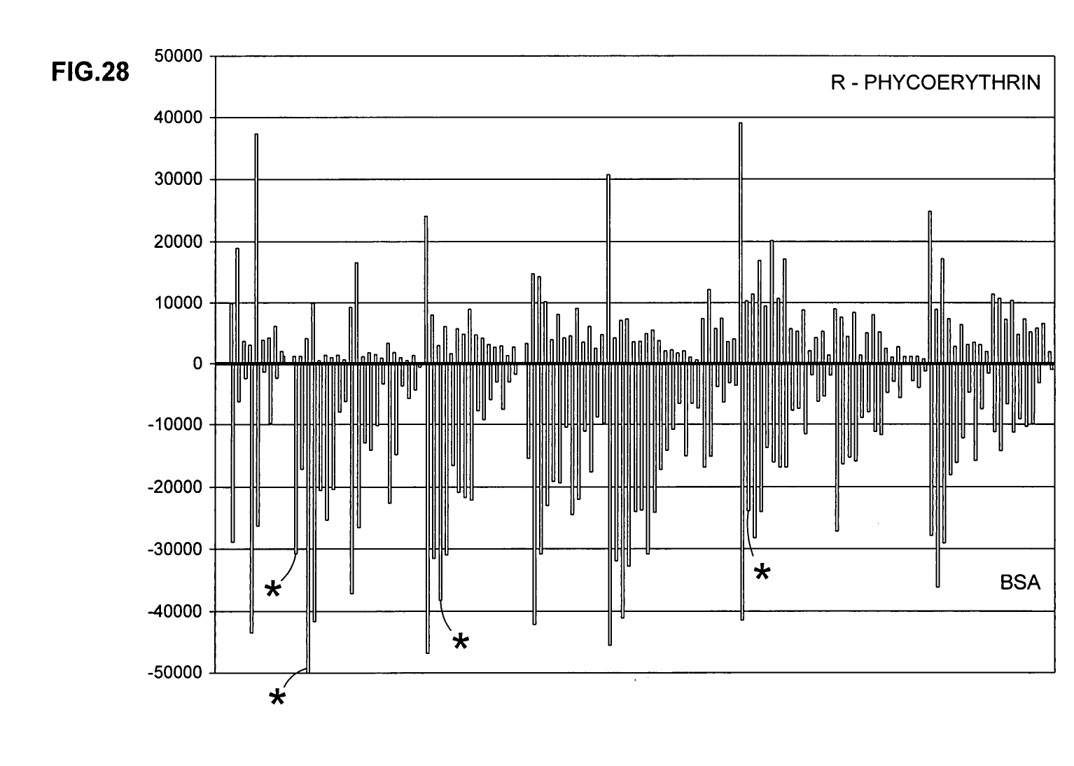

Candidate receptors can also be displayed in a chart comparing binding signal strength with other candidate receptors including the same building blocks. For example, each candidate receptor can be located on a chart in which candidate receptors are grouped by the building blocks that they contain (see, e.g., FIG. 28). Again, signal strength can be illustrated as a third dimension. Those candidate receptors showing the greatest binding can then be found and evaluated with respect to candidate receptors including the same building blocks.

According to the present method, screening candidate artificial receptors against a test ligand can yield one or more lead artificial receptors. One or more lead artificial receptors can be a working artificial receptor. That is, the one or more lead artificial receptors can be useful for detecting the ligand of interest as is. The method can then employ the one or more artificial receptors as a working artificial receptor for monitoring or detecting the test ligand. Alternatively, the one or more lead artificial receptors can be employed in the method for developing a working artificial receptor. For example, the one or more lead artificial receptors can provide structural or other information useful for designing or screening for an improved lead artificial receptor or a working artificial receptor. Such designing or screening can include making and testing additional candidate artificial receptors including combinations of a subset of building blocks, a different set of building blocks, or a different number of building blocks.

In certain embodiments, the method of the present invention can employ a smaller number of spots formed by combinations of a subset of the total building blocks and/or smaller groups of the building blocks. For example, the present method can employ an array including the number of spots formed by combinations of 81 building blocks in groups of 2 and/or 3. Then a smaller number of building blocks indicated by test compound binding, for example 36 building blocks, can be tested in a microarray with spots including larger groups, for example 4, of the building blocks. Each set of microarrays can employ a different support matrix, lawn, or functionalized lawn. Such methods are schematically illustrated in FIG. 10.

For example, FIG. 10 illustrates that a single slide with the 3,240 combinations of 2 building blocks that can be produced from a set of 81 building blocks can be used to define a subset of the building blocks. This subset of, e.g., 25, building blocks (which can be derived from a 5×5 matrix of the results employing combinations of 2 building blocks), can be used to produce an additional 2,300 combinations of 3 building blocks and/or 12,650 combinations of 4 building blocks. These combinations from the subset can be screened to define the optimum receptor configuration. The method can also include using combinations of building blocks in different ratios in spots.

On a macro scale, an artificial receptor presented as a spot or region including a plurality of building blocks has the plurality of building blocks distributed randomly throughout the spot or region. On a molecular scale, the distribution may not be random and even. For example, any selected group of only 2-10 building blocks may include a greater number of a particular building block or a particular arrangement of building blocks with respect to one another. A spot or region with a random distribution makes a useful artificial receptor according to the present invention. Particular assortments of building blocks found in a random distribution can also make useful artificial receptors.

An artificial receptor can include a particular assortment of a combination of 2, 3, 4, or more building blocks. Such an assortment can be visualized as occupying positions on the surface of a support. A combination of 2, 3, 4, or more building blocks can have each of the different building blocks in distinct positions relative to one another. For example, building block 1 can be adjacent to any of building blocks 2, 3, or 4. This can be illustrated by considering the building blocks at the vertices of a polygon. For example, FIG. 8 illustrates positional isomers of 4 different building blocks at the vertices of a quadrilateral. By way of further example, 2 building blocks can be envisioned as located at points on a line, 3 building blocks can be envisioned as located at vertices of a triangle, 5 building blocks can be envisioned as located at the vertices of a pentagon, and so on.

In an embodiment of the method, a candidate artificial receptor can be optimized to a lead or working artificial receptor by making one or more of the positional isomers and determining its ability to bind the test ligand of interest. Advantageously, the positional isomers can be made on a scaffold (FIG. 8). Scaffold positional isomer artificial receptors can be made, for example, on a scaffold with multiple functional groups that can be protected and deprotected by orthogonal chemistries. The scaffold positional isomer lead artificial receptors can be evaluated by any of a variety of methods suitable for evaluating binding of ligands to scaffold receptors. For example, the scaffold lead artificial receptors can be chromatographed against immobilized test ligand.

In an embodiment, the method of using an artificial receptor includes contacting a first heterogeneous molecular array with a test ligand. The array can include a support and a plurality of spots of building blocks attached to the support. In the array, each spot of building blocks can include a plurality of building blocks with each building block being coupled to the support. The method includes detecting binding of a test ligand to one or more spots; and selecting one or more of the binding spots as the artificial receptor.

In this embodiment, the building blocks in the array can define a first set of building blocks, and the plurality of building blocks in each binding spot defines one or more selected binding combinations of building blocks. The first set of building blocks can include or be a subset of a larger set of building blocks. In an embodiment, the spots of building blocks can include 2, 3, or 4 building blocks. The first set can be immobilized using a first support matrix, a first lawn, or a first functionalized lawn.

In the method, the artificial receptor can include or be one or more lead artificial receptors. In the method, the artificial receptors can include or be one or more working artificial receptors.

This embodiment of the method can also include determining the combinations of building blocks in the one or more binding spots. These combinations can be used as the basis for developing one or more developed combinations of building blocks distinct from those in the one or more selected combinations of building blocks. This embodiment continues with contacting the test ligand with a second heterogeneous molecular array comprising a plurality of spots, each spot comprising a developed combination of building blocks; detecting binding of a test ligand to one or more spots of the second heterogeneous molecular array; and selecting one or more of the spots of the second heterogeneous molecular array as the artificial receptor. The second set can be immobilized using a second support matrix, a second lawn, or a second functionalized lawn different from those used with the first set.

In this embodiment, the building blocks in the second heterogeneous molecular array define a second set of building blocks. The first set of building blocks can include or be a subset of a larger set of building blocks and/or the second subset of building blocks can include or define a subset of the larger set of building blocks. Advantageously, the first subset is not equivalent to the second subset. In an embodiment, the spots of the second heterogeneous molecular array can include 3, 4, or 5 building blocks, and/or the spots of the second heterogeneous molecular array can include more building blocks than the binding spots.

The artificial receptor can include or be a lead artificial receptor. The artificial receptor can include or be one or more working artificial receptors. The method can also include varying the structure of the lead artificial receptor to increase binding speed or binding affinity of the test ligand.

In an embodiment, the method includes identifying the plurality of building blocks making up the artificial receptor. The identified plurality of building blocks can then be coupled to a scaffold molecule to make a scaffold artificial receptor. This scaffold artificial receptor can be evaluated for binding of the test ligand. In an embodiment, coupling the identified plurality of building blocks to the scaffold can include making a plurality of positional isomers of the building blocks on the scaffold. Evaluating the scaffold artificial receptor can then include comparing the plurality of the scaffold positional isomer artificial receptors. In this embodiment, one or more of the scaffold positional isomer artificial receptors can be selected as one or more lead or working artificial receptors.

In an embodiment, the method includes screening a test ligand against an array including one or more spots that function as controls for validating or evaluating binding to artificial receptors of the present invention. In an embodiment, the method includes screening a test ligand against one or more regions, tubes, or wells that function as controls for validating or evaluating binding to artificial receptors of the present invention. Such a control spot, region, tube, or well can include no building block, only a single building block, only functionalized lawn, or combinations thereof.

Working Receptor Systems

In an embodiment, a working artificial receptor or working artificial receptor complex can be incorporated into a system or device for detecting a ligand of interest. Binding of a ligand of interest to a working artificial receptor or complex can produce a detectable signal, for example, through mechanisms and properties such as scattering, absorbing or emitting light, producing or quenching fluorescence or luminescence, producing or quenching an electrical signal, and the like. Spectroscopic detection methods include use of labels or enzymes to produce light for detection by optical sensors or optical sensor arrays. The light can be ultraviolet, visible, or infrared light, which can be produced and/or detected through fluorescence, fluorescence polarization, chemiluminescence, bioluminescence, or chemibioluminescence. Systems and methods for detecting electrical conduction, and changes in electrical conduction, include ellipsometry, surface plasmon resonance, capacitance, conductometry, surface acoustic wave, quartz crystal microbalance, love-wave, infrared evanescent wave, enzyme labels with electrochemical detection, nanowire field effect transistors, MOSFETS—metal oxide semiconductor field effect transistors, CHEMFETS—organic membrane metal oxide semiconductor field effect transistors, ICP—intrinsically conducting polymers, FRET—fluorescence resonance energy transfer.

Apparatus that can detect such binding to or signal from a working artificial receptor or complex includes UV, visible or infrared spectrometer, fluorescence or luminescence spectrometer, surface plasmon resonance, surface acoustic wave or quartz crystal microbalance detectors, pH, voltammetry or amperometry meters, radioisotope detector, or the like.

In such an apparatus, a working artificial receptor or complex can be positioned on a light fiber to provide a detectable signal, such as an increase or decrease in transmitted light, reflected light, fluorescence, luminescence, or the like. The detectable signal can originate from, for example, a signaling moiety incorporated into the working artificial receptor or complex or a signaling moiety added to the working artificial receptor. The signal can also be intrinsic to the working artificial receptor or to the ligand of interest. The signal can come from, for example, the interaction of the ligand of interest with the working artificial receptor, the interaction of the ligand of interest with a signaling moiety which has been incorporated into the working artificial receptor, into the light fiber, onto the light fiber.

In an embodiment of the system, more than one working artificial receptor, arranged as regions or spots in an array, is on the surface of a support, such as a glass plate. The ligand or ligands of interest or a sample suspected of containing the ligand or ligands of interest (e.g., a sample containing a mixture of DNA segments or fragments, proteins or protein fragments, carbohydrates or carbohydrate fragments, or the like) is brought into contact with the working artificial receptors or array. Contact can be achieved by addition of a solution of the ligand or ligands of interest or a sample suspected of containing the ligand or ligands of interest. A detectable fluorescence signal can be produced by a signaling moiety incorporated into the working artificial receptor array or a signaling moiety which is added to the ligand or ligands of interest or the sample suspected of containing the ligand or ligands of interest. The fluorescent moieties produce a signal for each working artificial receptor in the array, which produces a pattern of signal response which is characteristic of the composition of the sample of interest.

In an embodiment of the system, more than one working artificial receptor, arranged as regions or spots in an array, is on a support, such as a glass or plastic surface. The surface can be incorporated onto the signaling surfaces of one or more surface plasmon resonance detectors. The ligands of interest or a sample suspected of containing the ligands of interest (e.g., a sample containing a mixture of DNA segments or fragments, proteins or protein fragments, carbohydrates or carbohydrate fragments, or the like) is brought into contact with the working artificial receptors or array. Contacting can be accomplished by addition of a solution of the ligands of interest or a sample suspected of containing the ligands of interest. Detectable electrical signals can be produced by binding of the ligands of interest to the working artificial receptors array on the surface of the surface plasmon resonance detectors. Such detectors produce a signal for each working artificial receptor in the array, which produces a pattern of signal response, which is characteristic of the composition of the sample of interest.

In an embodiment of the system, the working artificial receptor is on a support such as the inner surface of a test tube, microwell, capillary, microchannel, or the like. The ligand of interest or a sample suspected of containing the ligand of interest is brought into contact with the working artificial receptor or complex by addition of a solution containing the ligand of interest or a sample suspected of containing the ligand of interest. A detectable colorimetric, fluorometric, radiometric, or the like, signal is produced by a calorimetric, enzyme, fluorophore, radioisotope, metal ion, or the like, labeled compound or conjugate of the ligand of interest. This labeled moiety can be reacted with the working artificial receptor or complex in competition with the solution containing the ligand of interest or the sample suspected of containing the ligand of interest.

In an embodiment of the system, the working artificial receptor is on a support such as the surface of a surface acoustic wave or quartz crystal microbalance or surface plasmon resonance detector. The ligand of interest or a sample suspected of containing the ligand of interest can be brought into contact with the working artificial receptor or complex by exposure to a stream of air, to an aerosol, or to a solution containing the ligand of interest or a sample suspected of containing the ligand of interest. A detectable electrical signal can be produced by the interaction of the ligand of interest with the working artificial receptor or complex on the active surface of the surface acoustic wave or quartz crystal microbalance or surface plasmon resonance detector.

In an embodiment of the system, the more than one working artificial receptor, arranged as a series of discrete areas or spots or zones or the like, is on the surface of a light fiber. The ligand of interest or a sample suspected of containing the ligand of interest can be brought into contact with the working artificial receptor or complex by exposure to a stream of air, to an aerosol, or to a solution containing the ligand of interest or a sample suspected of containing the ligand of interest. A detectable calorimetric, fluorometric, or like signal can be produced by a label incorporated into the light fiber surface. The colorimetric or fluorogenic signal can be intrinsic to the ligand, or can be an inherent calorimetric or fluorogenic signal produced on binding of the ligand to the working artificial receptors.

An embodiment of the system, combines the artificial receptors with nanotechnology derived nanodevices to give the devices the ability to bind (“see”), bind and incorporate (“eat”), or modify (“use in manufacture”) the target material. In an embodiment of the system, the working artificial receptor is incorporated into or on a nanodevice. The ligand of interest or a sample suspected of containing the ligand of interest can be brought into contact with the working artificial receptor nanodevice by addition of the nanodevice to an air or water or soil or biological fluid or cell or biological tissue or biological organism or the like. A detectable signal can be produced by a suitable sensor on the nanodevice and a desired action like a radio signal or chemical reaction or mechanical movement or the like is produced by the nanodevice in response to the ligand of interest.

The present artificial receptors can be part of products used in: analyzing a genome and/or proteome; pharmaceutical development; detectors for any of the test ligands; drug of abuse diagnostics or therapy; hazardous waste analysis or remediation; toxic chemical agent alert or intervention; disease diagnostics or therapy; cancer diagnostics or therapy; toxic biological agent alert or intervention; food chain contamination analysis or remediation; and the like.

More specifically, the present artificial receptors can be used in products for identification of sequence specific small molecule leads; protein isolation and identification; identification of protein to protein interactions; detecting contaminants in food or food products; clinical analysis of food contaminants; clinical analysis of prostate specific antigen; clinical and field or clinical analysis of cocaine; clinical and field or clinical analysis of other drugs of abuse; other clinical analysis systems, home test systems, or field analysis systems; monitors or alert systems for toxic biological or chemical agents; and the like.

In an embodiment, the present artificial receptors can be employed in studies of proteomics. In such an embodiment, an array of candidate or working artificial receptors can be contacted with a mixture of peptides, polypeptides, and/or proteins. Each mixture can produce a characteristic fingerprint of binding to the array. In addition, identification of a specific receptor environment for a target peptide, polypeptide, and/or protein can be utilized for isolation and analysis of the target. That is, in yet another embodiment, a particular receptor surface can be employed for affinity purification methods, e.g. affinity chromatography.

In an embodiment, the present artificial receptors can be employed to form bioactive surfaces. For example, receptor surfaces can be used to specifically bind antibodies or enzymes.

In an embodiment, the present candidate artificial receptors can be employed to find non-nucleotide artificial receptors for individual DNA or RNA sequences.

In an embodiment, the present candidate artificial receptors can be employed to find receptor surfaces that bind proteins in a certain configuration or orientation. Many proteins (e.g. antibodies, enzymes, receptors) are stable and/or active in specific environments. Defined receptor surfaces can be used to produce binding environments that selectively retain or orient the protein for maximum stability and/or activity.

In an embodiment, the present candidate artificial receptors can be employed to find artificial receptors that do not bind selected molecules or compositions or that exhibit low friction. For example, an array of candidate artificial receptors can be surveyed to find artificial receptors that not bind to complex biological mixtures like blood serum. Non-binding surfaces can be made by coating with the selected artificial receptor. For example, surfaces can be made that are anti-filming or that have antimicrobial properties.

In an embodiment, the present candidate artificial receptors can be employed to find receptor surfaces that provide a spatially oriented binding surface for a stereospecific reaction. For example, an artificial receptor surface can bind a small molecule with particular functional groups exposed to the environment, and others obscured by the receptor. Such an artificial receptor surface can be employed in synthesis including chiral induction. For example, a substrate (e.g. a steroid) can be stereospecifically bound to the artificial receptor and present a particular moiety/sub-structure/“face” for reaction with a reagent in solution. Similarly, the artificial receptor surface can act as a protecting group where a reactive moiety of a molecule is “protected” by binding to the receptor surface so that a different moiety with similar reactivity can be transformed.

In an embodiment, the present candidate artificial receptors can be employed to find artificial receptors or receptor surfaces that act as an artificial enzyme. For example, such a receptor surface can be utilized as co-factor to bind a catalytic center and/or to orient the substrate for reaction.

In an embodiment, the present artificial receptors can be employed to form selective membranes. Such a selective membrane can be based on a molecular gate including an artificial receptor surface. For example, an artificial receptor surface can line the walls of pores in the membrane and either allow or block a target molecule from passing through the pores. For example, an artificial receptor surface can line the walls of pores in the membrane and act as “gatekeepers” on e.g. microcantilevers/molecular cantilevers to allow gate opening or closing on binding of the target.

In an embodiment, the present candidate artificial receptors can be employed to find artificial receptors for use on surfaces as intelligent materials. For example, the artificial receptor surface can act as a molecular electronic switch. In such a switch, binding of a target, which can be either an organic or an inorganic moiety, can act as an on/off gate for electron or ion flow.

Test Ligands

The test ligand can be any ligand for which binding to an array or surface can be detected. The test ligand can be a pure compound, a mixture, or a “dirty” mixture containing a natural product or pollutant. Such dirty mixtures can be tissue homogenate, biological fluid, soil sample, water sample, or the like.

Test ligands include prostate specific antigen, other cancer markers, insulin, warfarin, other anti-coagulants, cocaine, other drugs-of-abuse, markers for E. coli, markers for Salmonella sp., markers for other food-borne toxins, food-borne toxins, markers for Smallpox virus, markers for anthrax, markers for other toxic biological agents, pharmaceuticals and medicines, pollutants and chemicals in hazardous waste, toxic chemical agents, markers of disease, pharmaceuticals, pollutants, biologically important cations (e.g., potassium or calcium ion), peptides, carbohydrates, enzymes, bacteria, viruses, mixtures thereof, and the like. In certain embodiments, the test ligand can be at least one of small organic molecules, inorganic/organic complexes, metal ion, mixture of proteins, protein, nucleic acid, mixture of nucleic acids, mixtures thereof, and the like.

The present invention may be better understood with reference to the following examples. These examples are intended to be representative of specific embodiments of the invention, and are not intended as limiting the scope of the invention.

### EXAMPLES

### Example 1

### Synthesis of Building Blocks

Selected building blocks representative of the alkyl-aromatic-polar span of the an embodiment of the building blocks were synthesized and demonstrated effectiveness of these building blocks for making candidate artificial receptors. These building blocks were made on a framework that can be represented by tyrosine and included numerous recognition element pairs. These recognition element pairs were selected along the diagonal of Table 2, and include enough of the range from alkyl, to aromatic, to polar to represent a significant degree of the interactions and functional groups of the full set of 81 such building blocks.

Synthesis

Building block synthesis employed a general procedure outlined in Scheme 2, which specifically illustrates synthesis of a building block on a tyrosine framework with recognition element pair A4B4. This general procedure was employed for synthesis of building blocks including TyrA1B1 [1-1], TyrA2B2, TyrA2B4, TyrA2B6, TyrA2B8, TyrA4B2, TyrA4B4, TyrA4B6, TyrA4B8, TyrA6B2, TyrA6B4, TyrA6B6, TyrA6B8, TyrA8B2, TyrA8B4, TyrA8B6, TyrA8B8, and TyrA9B9, respectively. 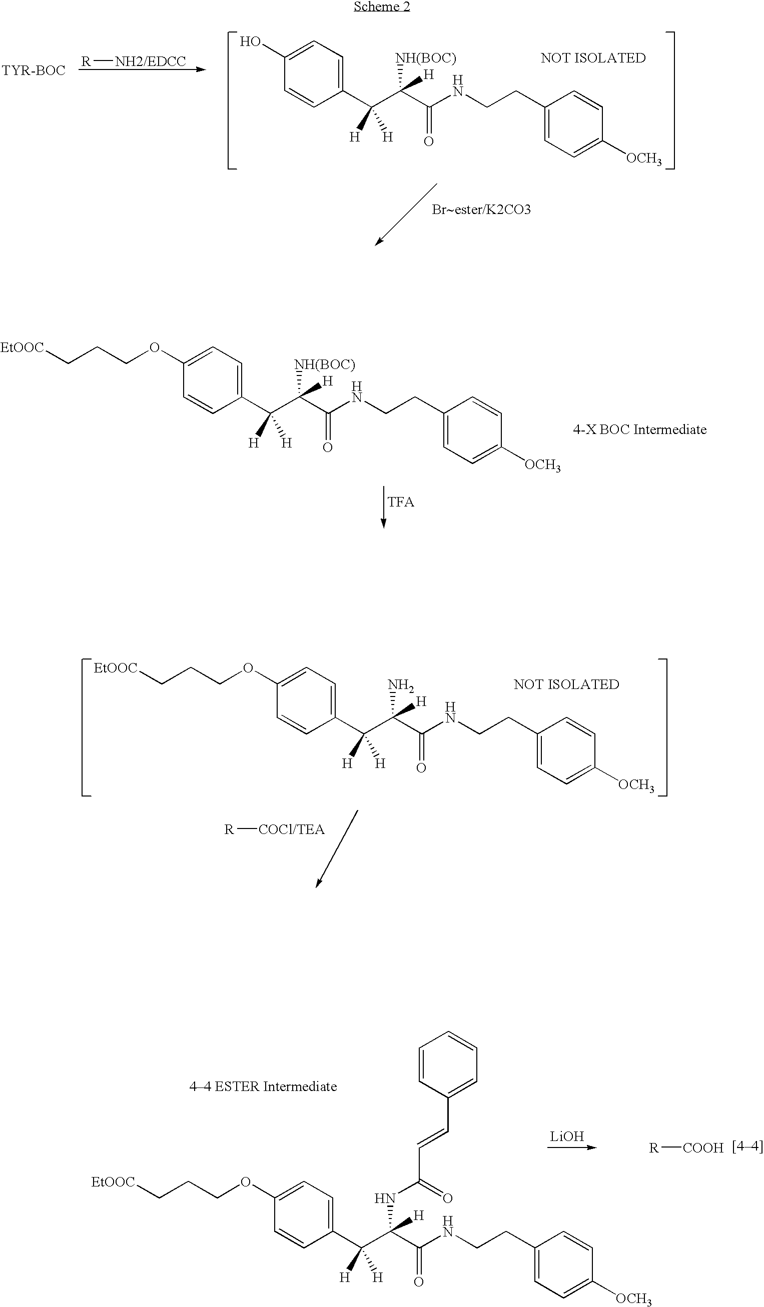
Results

Synthesis of the desired building blocks proved to be generally straightforward. These syntheses illustrate the relative simplicity of preparing the building blocks with 2 recognition elements having different structural characteristics or structures (e.g. A4B2, A6B3, etc.) once the building blocks with corresponding recognition elements (e.g. A2B2, A4B4, etc) have been prepared via their X BOC intermediate.

The conversion of one of these building blocks to a building block with a lipophilic linker can be accomplished by reacting the activated building block with, for example, dodecyl amine.

### Example 2

### Preparation and Evaluation of Microarrays of Candidate Artificial Receptors

Microarrays of candidate artificial receptors were made and evaluated for binding several protein ligands. The results obtained demonstrate the 1) the simplicity with which microarrays of candidate artificial receptors can be prepared, 2) binding affinity and binding pattern reproducibility, 3) significantly improved binding for building block heterogeneous receptor environments when compared to the respective homogeneous controls, and 4) ligand distinctive binding patterns (e.g., working receptor complexes).

Materials and Methods

Building blocks were synthesized and activated as described in Example 1. The building blocks employed in this example were TyrA1B1 [1-1], TyrA2B2, TyrA2B4, TyrA2B6, TyrA4B2, TyrA4B4, TyrA4B6, TyrA6B2, TyrA6B4, and TyrA6B6. The abbreviation for the building block including a linker, a tyrosine framework, and recognition elements AxBy is TyrAxBy.

Microarrays for the evaluation of the 130 n=2 and n=3, and for evaluation of the 273 n=2, n=3, and n=4, candidate receptor environments were prepared as follows by modifications of known methods. Briefly: Amine modified (amine “lawn”; SuperAmine Microarray plates) microarray plates were purchased from Telechem Inc., Sunnyvale, Calif. (www.arrayit.com). These plates were manufactured specifically for microarray preparation and had a nominal amine load of 2-4 amines per square nm according to the manufacturer. The CAM microarrays were prepared using a pin microarray spotter instrument from Telechem Inc. (SpotBot™ Arrayer) typically with 200 um diameter spotting pins from Telechem Inc. (Stealth Micro Spotting Pins, SMP6) and 400-420 um spot spacing.

The 9 building blocks were activated in aqueous dimethylformamide (DMF) solution as described above. For preparing the 384-well feed plate, the activated building block solutions were diluted 10-fold with a solution of DMF/H₂O/PEG400 (90/10/10, v/v/v; PEG400 is polyethylene glycol nominal 400 FW, Aldrich Chemical Co., Milwaukee, Wis.). These stock solutions were aliquotted (10 μl per aliquot) into the wells of a 384-well microwell plate (Telechem Inc.). A separate series of controls were prepared by aliquotting 10 μl of building block with either 10 μl or 20 μl of the activated [1-1] solution. The plate was covered with aluminum foil and placed on the bed of a rotary shaker for 15 minutes at 1,000 RPM. This master plate was stored covered with aluminum foil at −20° C. when not in use.

For preparing the 384-well SpotBot™ plate, a well-to-well transfer (e.g. A-1 to A-1, A-2 to A-2, etc.) from the feed plate to a second 384-well plate was performed using a 4 μl transfer pipette. This plate was stored tightly covered with aluminum foil at −20° C. when not in use. The SpotBot™ was used to prepare up to 13 microarray plates per run using the 4 μl microwell plate. The SpotBot™ was programmed to spot from each microwell in quadruplicate. The wash station on the SpotBot™ used a wash solution of EtOH/H₂O (20/80, v/v). This wash solution was also used to rinse the microarrays on completion of the SpotBot™ printing run. The plates were given a final rinse with deionized (DI) water, dried using a stream of compressed air, and stored at room temperature.

Certain of the microarrays were further modified by reacting the remaining amines with succinic anhydride to form a carboxylate lawn in place of the amine lawn.

The following test ligands and labels were used in these experiments:
1) r-Phycoerythrin, a commercially available and intrinsically fluorescent protein with a FW of 2,000,000.
2) Ovalbumin labeled with the Alexa™ fluorophore (Molecular Probes Inc., Eugene, Oreg.).
3) BSA, bovine serum albumin, labeled with activated Rhodamine (Pierce Chemical, Rockford, Ill.) using the known activated carboxylprotocol. BSA has a FW of 68,000; the material used for this study had ca. 1.0 rhodamine per BSA.
4) Horseradish peroxidase (HRP) modified with extra amines and labeled as the acetamide derivative or with a 2,3,7,8-tetrachlorodibenzodixoin derivative were available through known methods. Fluorescence detection of these HRP conjugates was based on the Alexa 647-tyramide kit available from Molecular Probes, Eugene, Oreg.
5) Cholera toxin.

Microarray incubation and analysis was conducted as follows: For test ligand incubation with the microarrays, solutions (e.g. 500 μl) of the target proteins in PBS-T (PBS with 20 μl/L of Tween-20) at typical concentrations of 10, 1.0 and 0.1 μg/ml were placed onto the surface of a microarray and allowed to react for, e.g., 30 minutes. The microarray was rinsed with PBS-T and DI water and dried using a stream of compressed air.

The incubated microarray was scanned using an Axon Model 4200A Fluorescence Microarray Scanner (Axon Instruments, Union City, Calif.). The Axon scanner and its associated software produce a false color 16-bit image of the fluorescence intensity of the plate. This 16-bit data is integrated using the Axon software to give a Fluorescence Units value (range 0-65,536) for each spot on the microarray. This data is then exported into an Excel file (Microsoft) for further analysis including mean, standard deviation and coefficient of variation calculations.

Results

The CARA™: Combinatorial Artificial Receptor Array™ concept has been demonstrated using a microarray format. A CARA microarray based on N=9 building blocks was prepared and evaluated for binding to several protein and substituted protein ligands. This microarray included 144 candidate receptors (18 n=1 controls plus 6 blanks; 36 n=2 candidate receptors; 84 n=3 candidate receptors). This microarray demonstrated: 1) the simplicity of CARA microarray preparation, 2) binding affinity and binding pattern reproducibility, 3) significantly improved binding for building block heterogeneous receptor environments when compared to the respective homogeneous controls, and 4) ligand distinctive binding patterns.

Reading the Arrays

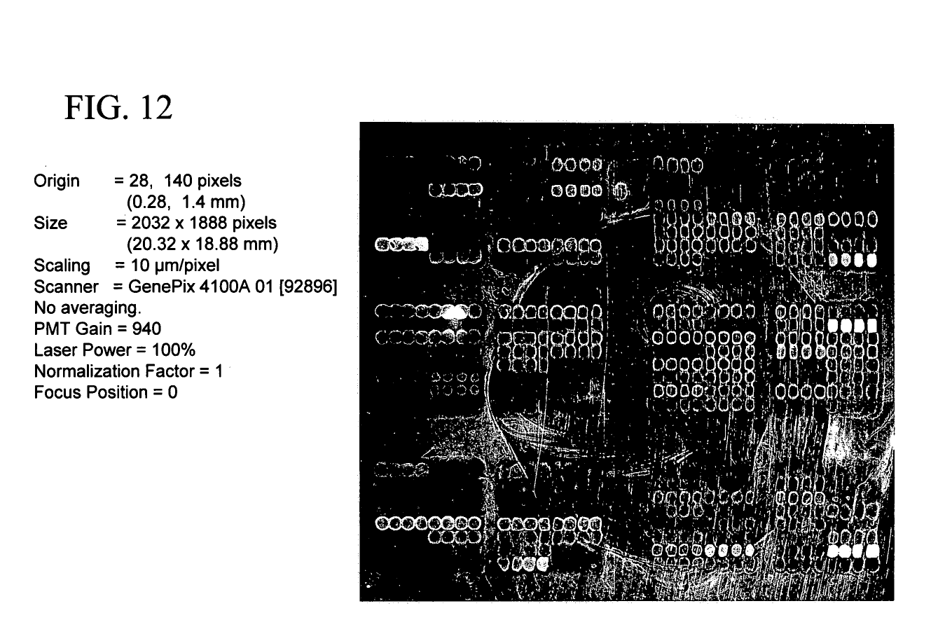

A typical false color/gray scale image of a microarray that was incubated with 2.0 μg/ml r-phycoerythrin is shown in FIG. 12. This image illustrates that the processes of both preparing the microarray and probing it with a protein test ligand produced the expected range of binding as seen in the visual range of relative fluorescence from dark to bright spots.

The starting point in analysis of the data was to take the integrated fluorescence units data for the array of spots and normalize to the observed value for the [1-1] building block control. Subsequent analysis included mean, standard deviation and coefficient of variation calculations. Additionally, control values for homogeneous building blocks were obtained from the building block plus [1-1] data.

First Set of Experiments

The following protein ligands were evaluated for binding to the candidate artificial receptors in the microarray. The resulting Fluorescence Units versus candidate receptor environment data is presented in both a 2D format where the candidate receptors are placed along the X-axis and the Fluorescence Units are shown on the Y-axis and a 3D format where the Candidate Receptors are placed in an X-Y format and the Fluorescence Units are shown on the Z-axis. A key for the composition of each spot was developed (not shown). A key for the building blocks in each of the 2D and 3D representations of the results was also developed (not shown). The data presented are for 1-2 μg/ml protein concentrations.

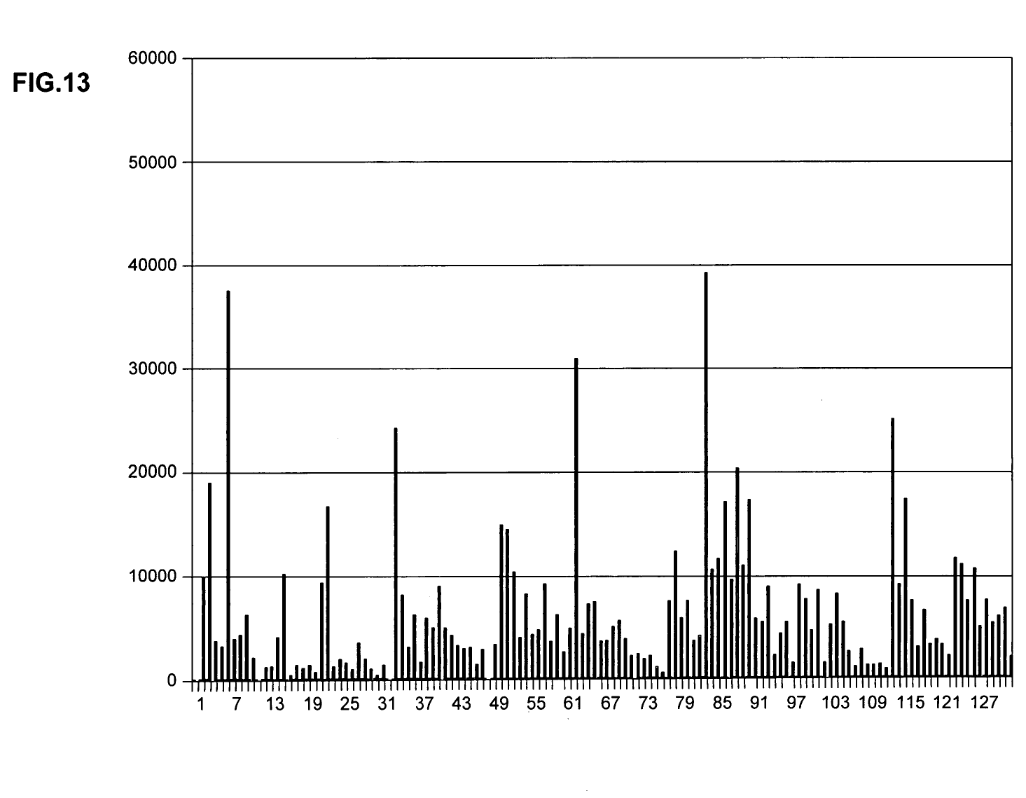

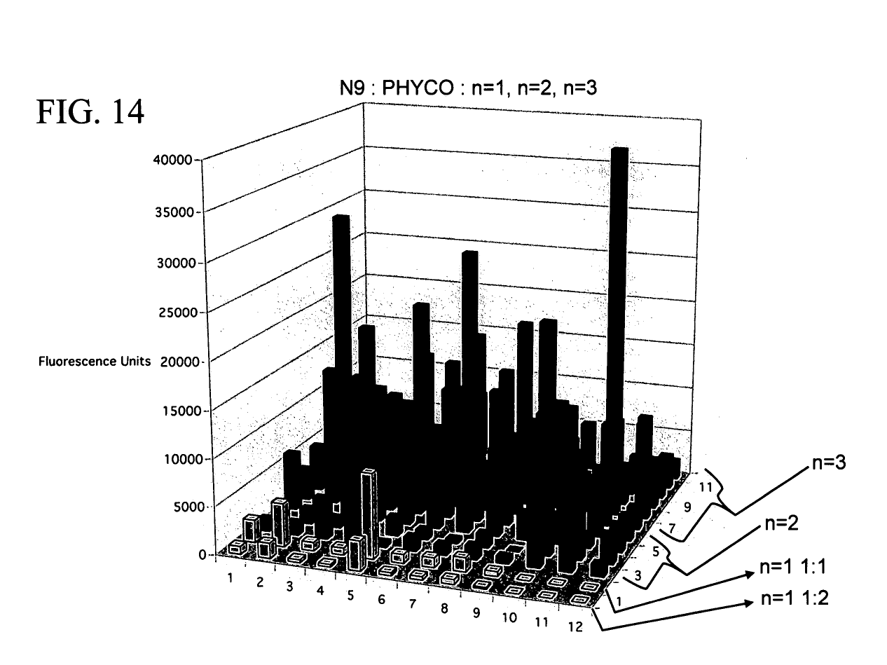

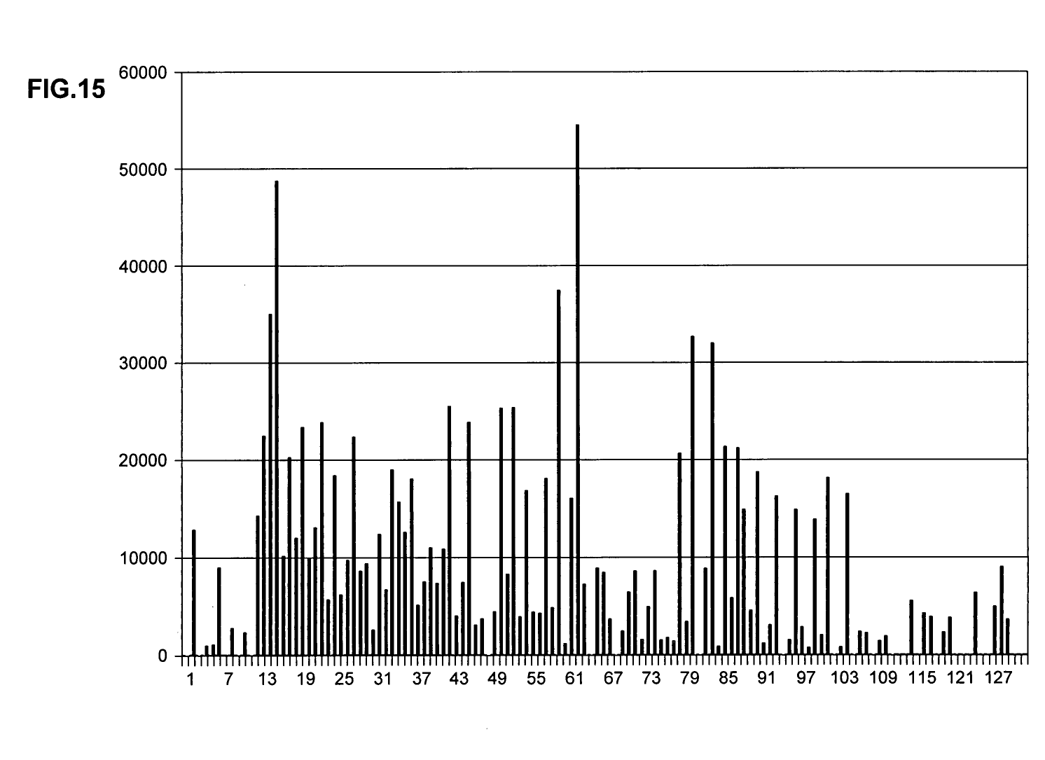

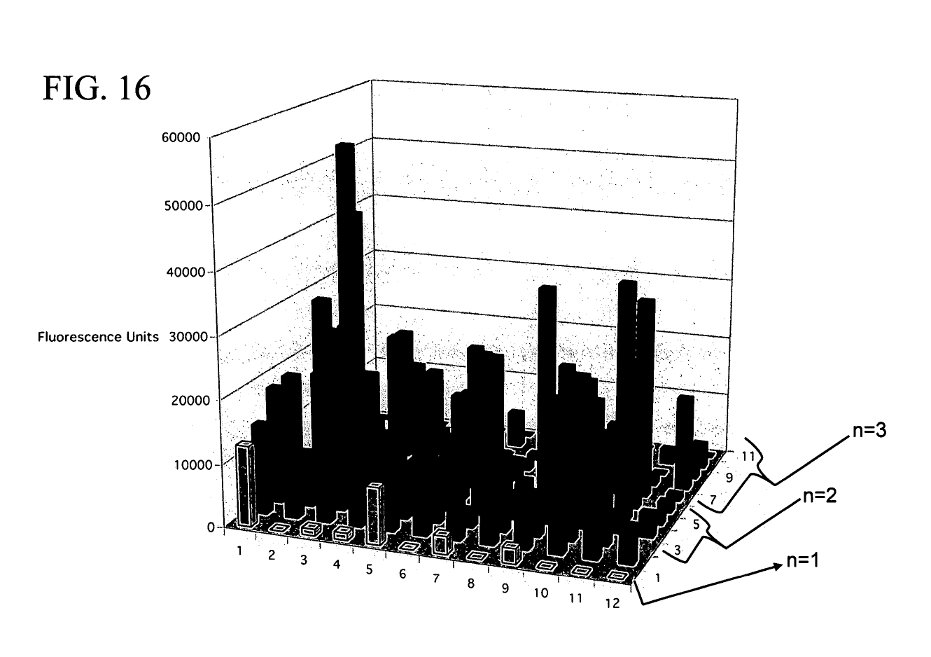

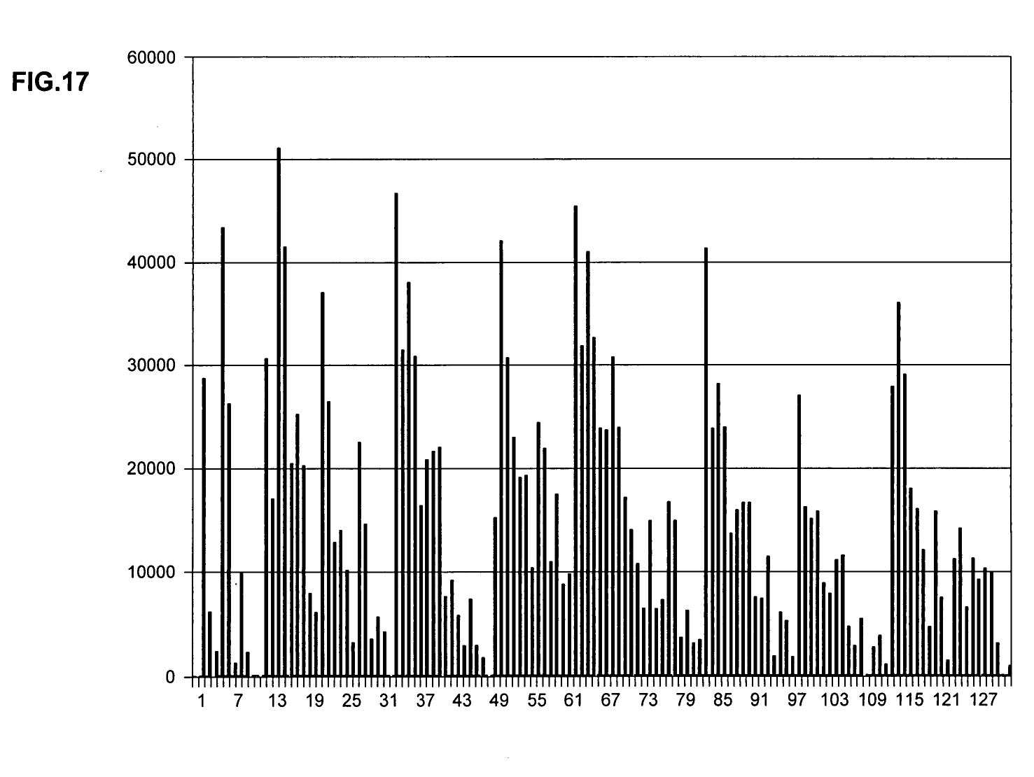

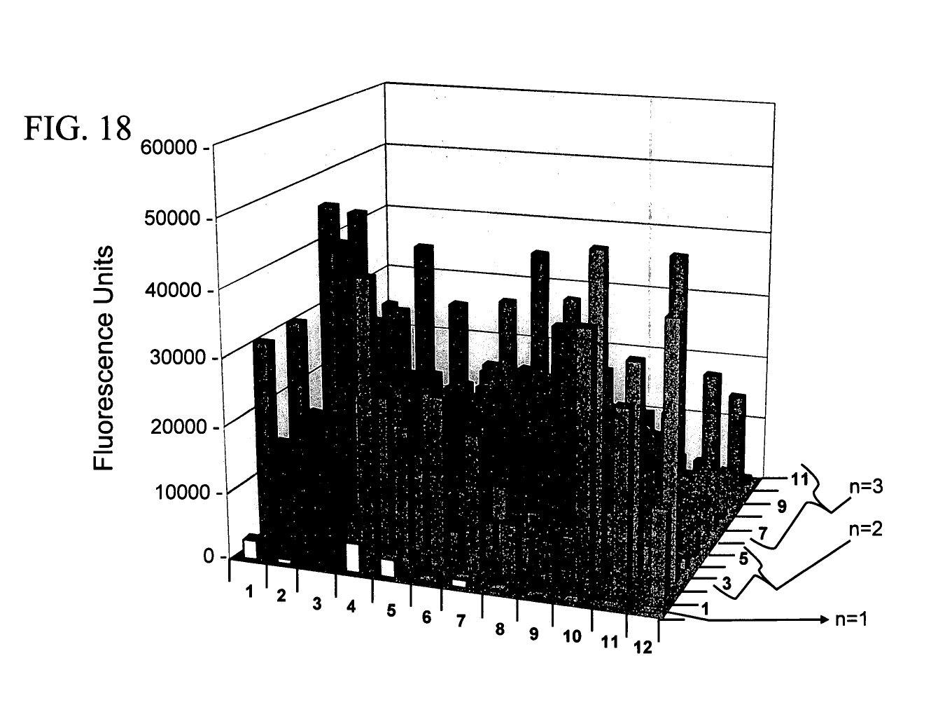

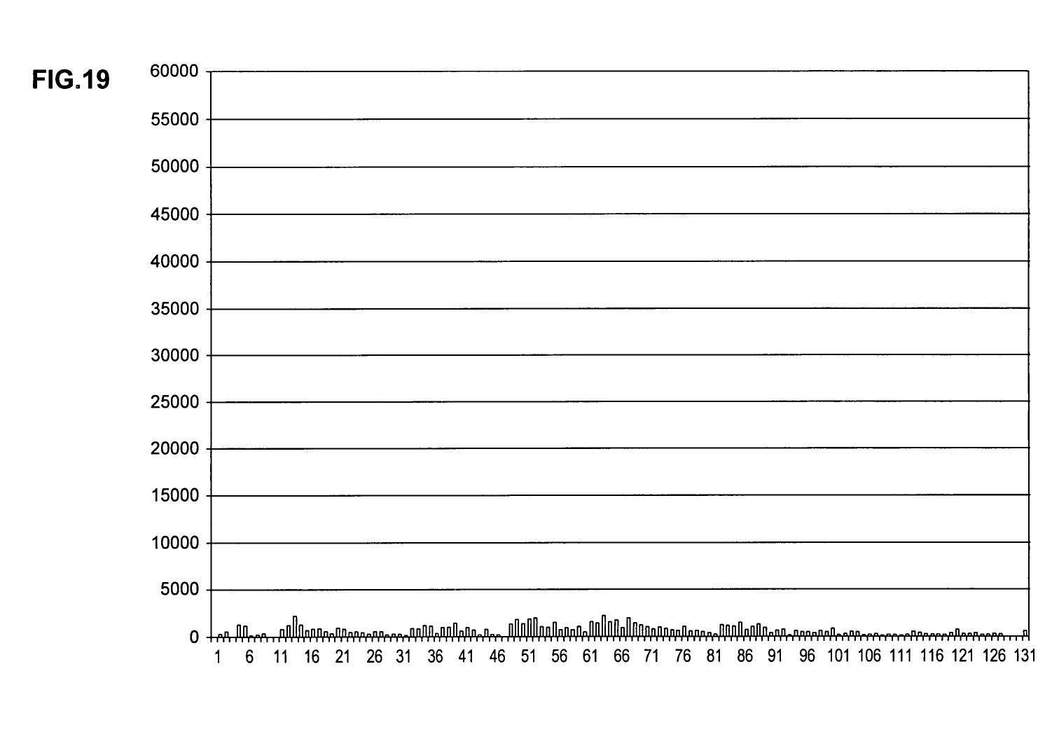

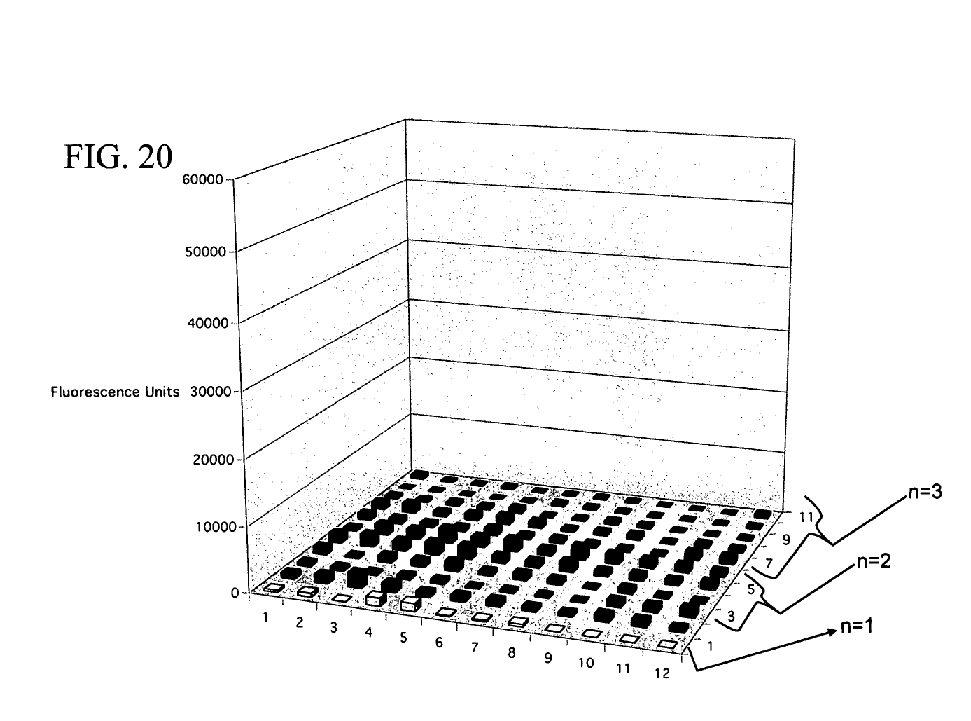

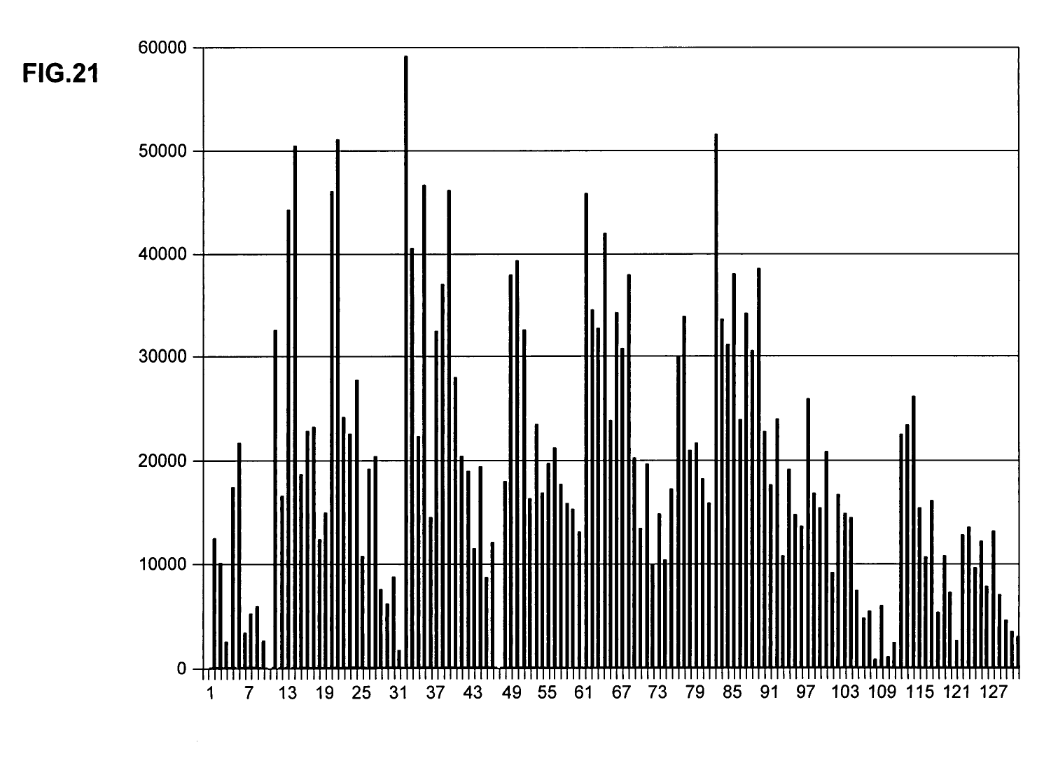

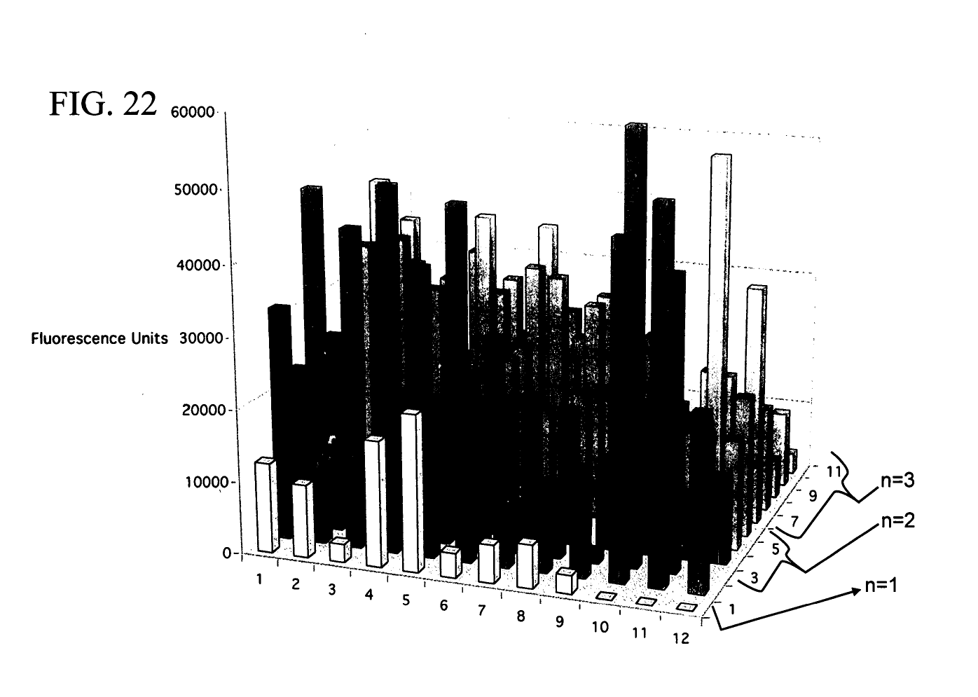

FIGS. 13 and 14 illustrate binding data for r-phycoerythrin (intrinsic fluorescence). FIGS. 15 and 16 illustrate binding data for ovalbumin (commercially available with fluorescence label). FIGS. 17 and 18 illustrate binding data for bovine serum albumin (labeled with rhodamine). FIGS. 19 and 20 illustrate binding data for HRP-NH-Ac (fluorescent tyramide read-out). FIGS. 21 and 22 illustrate binding data for HRP-NH-TCDD (fluorescent tyramide read-out).

These results demonstrate not only the application of the CARA microarray to candidate artificial receptor evaluation but also a few of the many read-out methods (e.g. intrinsic fluorescence, fluorescently labeled, in situ fluorescence labeling) which can be utilized for high throughput candidate receptor evaluation.

The evaluation of candidate receptors benefits from reproducibility. The following results demonstrate that the present microarrays provided reproducible ligand binding.

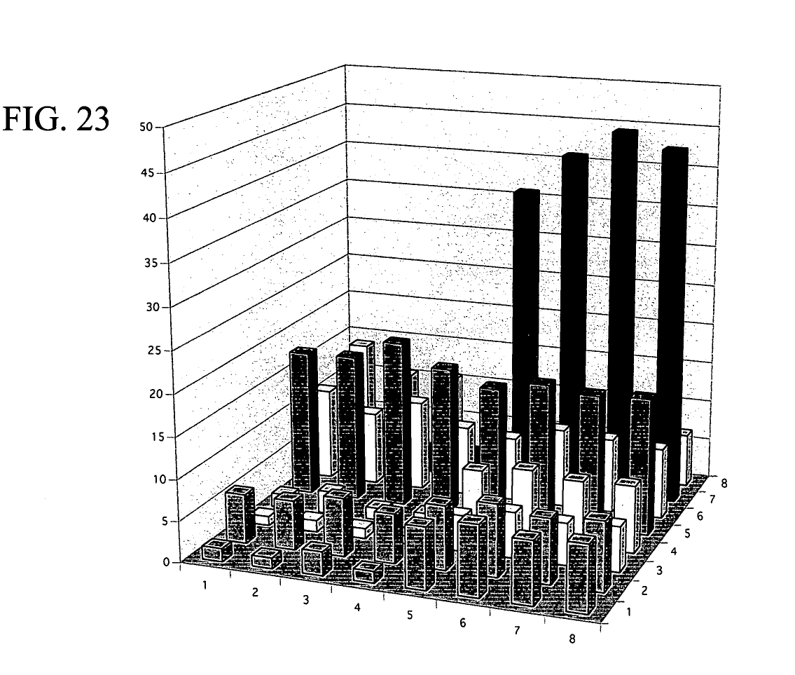

The microarrays were printed with each combination of building blocks spotted in quadruplicate. Visual inspection of a direct plot (FIG. 23) of the raw fluorescence data (from the run illustrated in FIG. 12) for one block of binding data obtained for r-phycoerythrin demonstrates that the candidate receptor environment “spots” showed reproducible binding to the test ligand. Further analysis of the r-phycoerythrin data (FIG. 12) led to only 9 out of 768 spots (1.2%) being deleted as outliers. Analysis of the r-phycoerythrin quadruplicate data for the entire array gives a mean standard deviation for each experimental quadruplicate set of 938 fluorescence units, with a mean coefficient of variation of 19.8%.

Although these values are acceptable, a more realistic comparison employed the standard deviation and coefficient of variation of the more strongly bound, more fluorescent receptors. The overall mean standard deviation unrealistically inflates the coefficient of variation for the weakly bound, less fluorescent receptors. The coefficient of variation for the 19 receptors with greater than 10,000 Fluorescent Units of bound target is 11.1%, which is well within the range required to produce meaningful binding data.

One goal of the CARA approach is the facile preparation of a significant number of candidate receptors through combinations of structurally simple building blocks. The following results establish that both the individual building blocks and combinations of building blocks have a significant, positive effect on test ligand binding.

The binding data illustrated in FIGS. 54-22 demonstrate that heterogeneous combinations of building blocks (n=2, n=3) are dramatically superior candidate receptors made from a single building block (n=1). For example, FIG. 14 illustrate both the diversity of binding observed for n=2, n=3 candidate receptors with fluorescent units ranging from 0 to ca. 40,000. These data also illustrate and the ca. 10-fold improvement in binding affinity obtained upon going from the homogeneous (n=1) to heterogeneous (n=2, n=3) receptor environments.

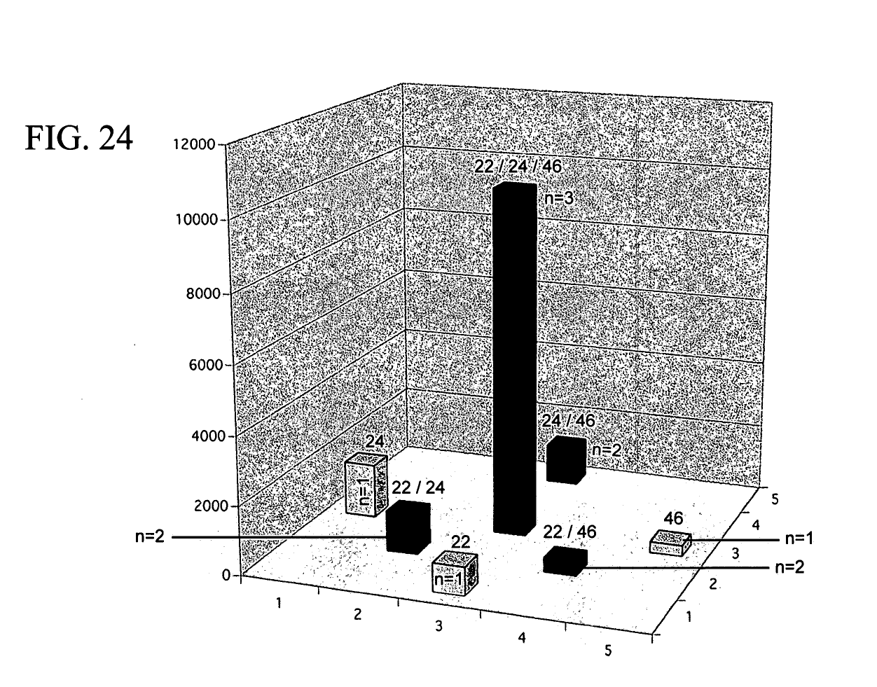

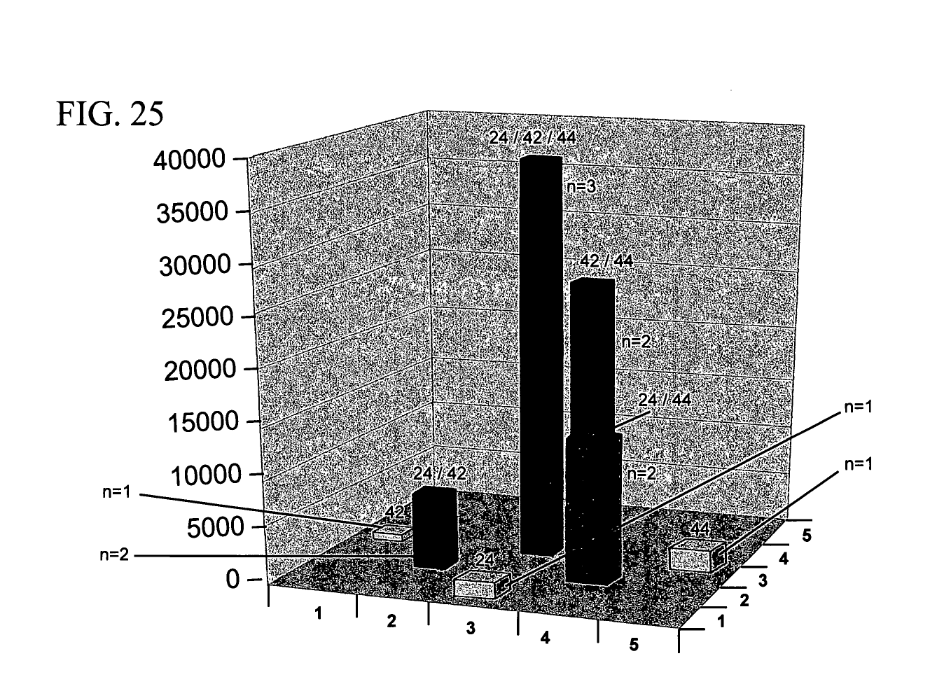

The effect of heterogeneous building blocks is most easily observed by comparing selected n=3 receptor environments candidate receptors including 1 or 2 of those building blocks (their n=2 and n=1 subsets). FIGS. 24 and 25 illustrate this comparison for two different n=3 receptor environments using the r-phycoerythrin data. In these examples, it is clear that progression from the homogeneous system (n=1) to the heterogeneous systems (n=2, n=3) produces significantly enhanced binding.

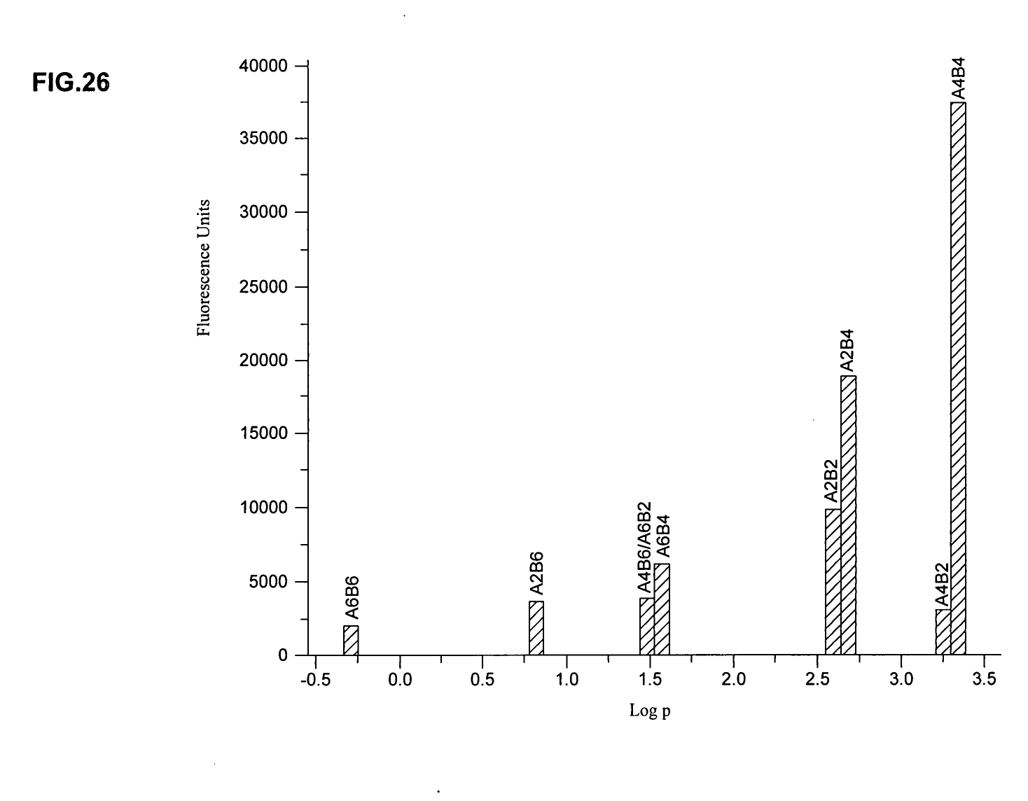

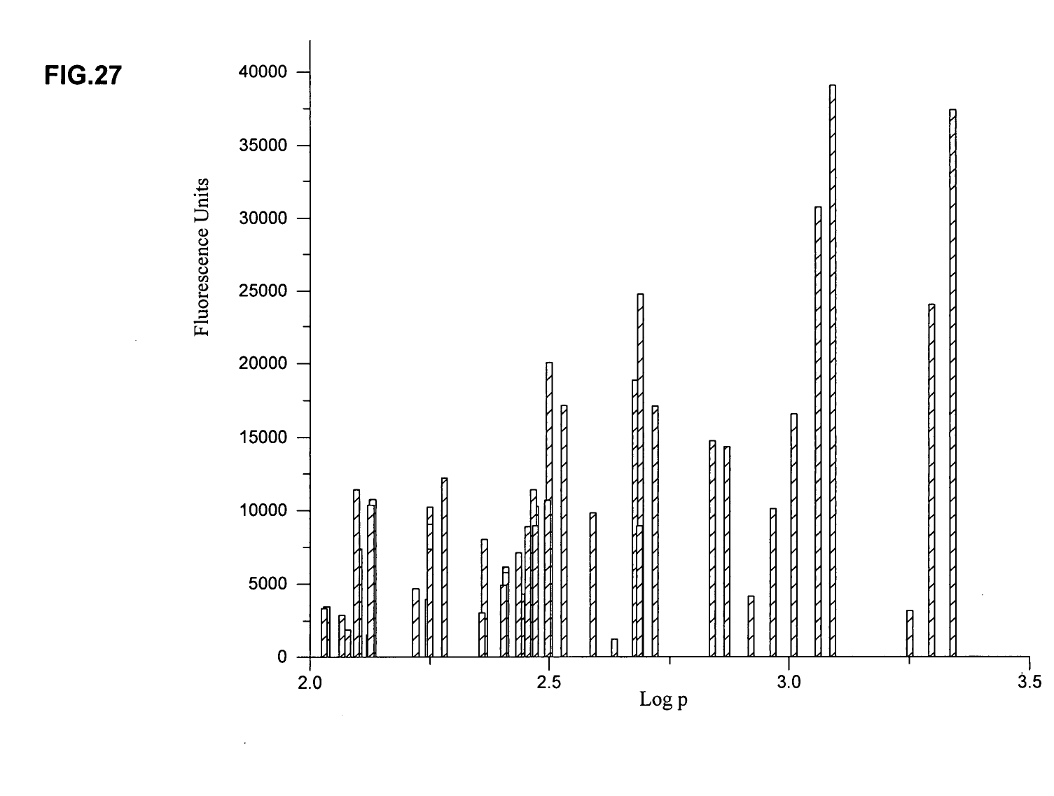

Although van der Waals interactions are an important part of molecular recognition, it is important to establish that the observed binding is not a simple case of hydrophobic/hydrophilic partitioning. That is, that the observed binding was the result of specific interactions between the individual building blocks and the target The simplest way to evaluate the effects of hydrophobicity and hydrophilicity is to compare building block logP value with observed binding. LogP is a known and accepted measure of lipophilicity, which can be measured or calculated by known methods for each of the building blocks. FIGS. 26 and 27 establish that the observed target binding, as measured by fluorescence units, is not directly proportional to building block logP. The plots in FIGS. 26 and 27 illustrate a non-linear relationship between binding (fluorescence units) and building block logP.

One advantage of the present methods and arrays is that the ability to screen large numbers of candidate receptor environments will lead to a combination of useful target affinities and to significant target binding diversity. High target affinity is useful for specific target binding, isolation, etc. while binding diversity can provide multiplexed target detection systems. This example employed a relatively small number of building blocks to produce ca. 120 binding environments. The following analysis of the present data clearly demonstrates that even a relatively small number of binding environments can produce diverse and useful artificial receptors.

The target binding experiments performed for this study used protein concentrations including 0.1 to 10 μg/ml. Considering the BSA data as representative, it is clear that some of the receptor environments readily bound 1.0 ug/ml BSA concentrations near the saturation values for fluorescence units (see, e.g., FIG. 18). Based on these data and the formula weight of 68,000 for BSA, several of the receptor environments readily bind BSA at ca. 15 picomole/ml or 15 nanomolar concentrations. Additional experiments using lower concentrations of protein (data not shown) indicate that, even with a small selection of candidate receptor environments, femptomole/ml or picomolar detection limits have been attained.

One goal of artificial receptor development is the specific recognition of a particular target. FIG. 28 compares the observed binding for r-phycoerythrin and BSA. Comparison of the overall binding pattern indicates some general similarities. However, comparison of specific features of binding for each receptor environment demonstrates that the two targets have distinctive recognition features as indicated by the (*) in FIG. 28.

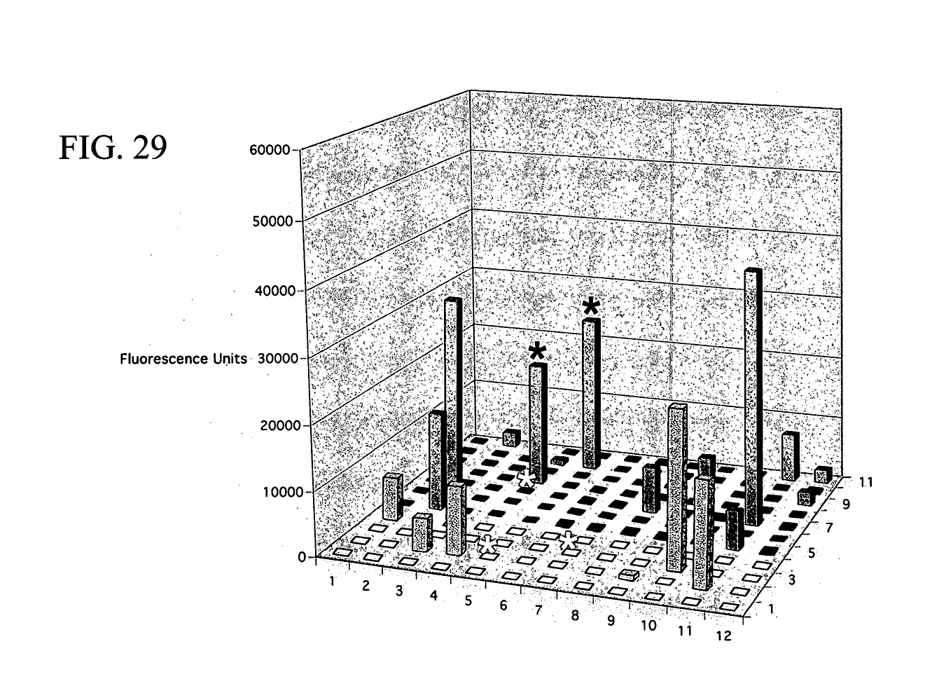

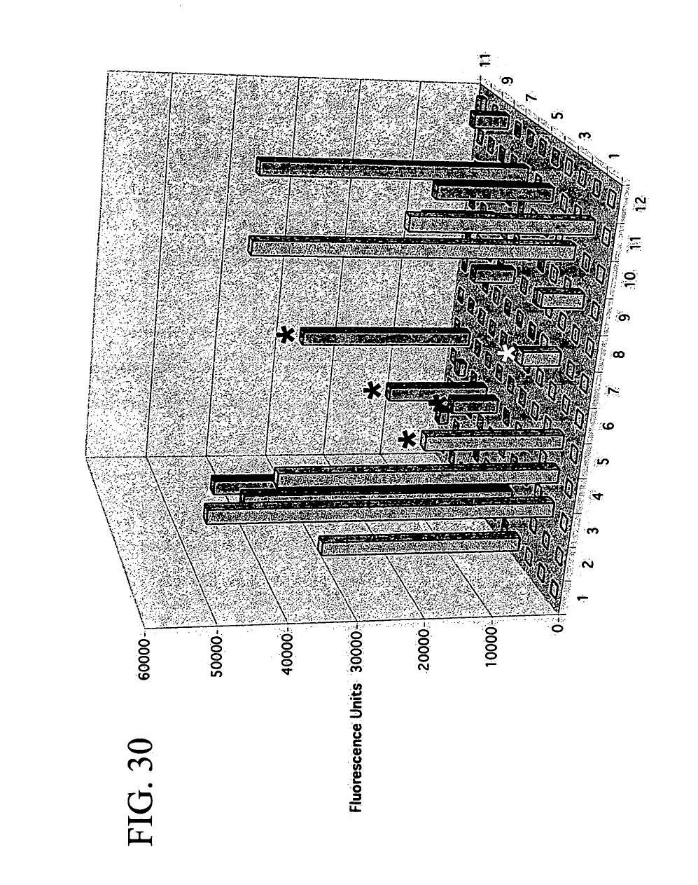

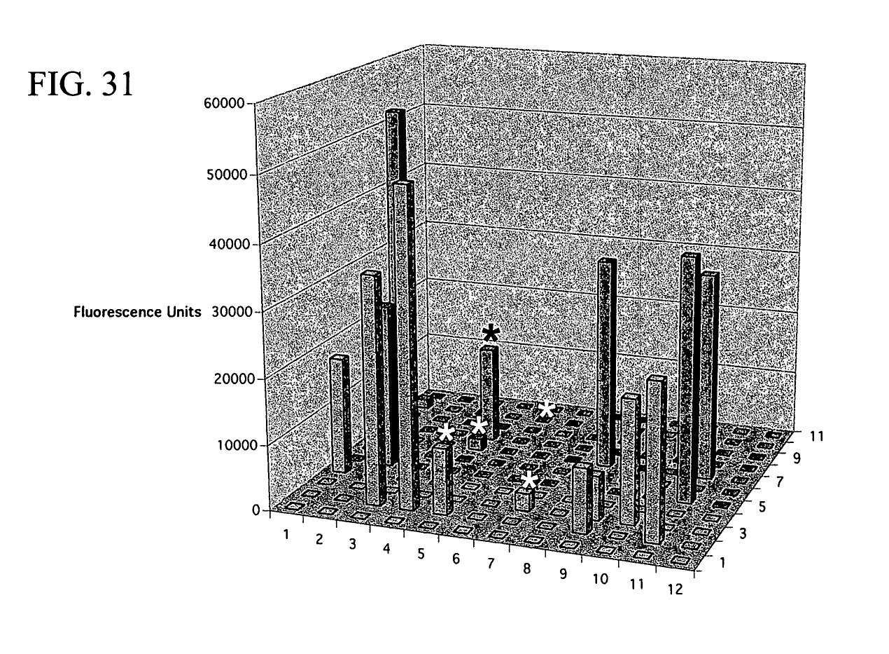

One goal of artificial receptor development is to develop receptors which can be used for the multiplexed detection of specific targets. Comparison of the r-phycoerythrin, BSA and ovalbumin data from this study (FIGS. 14, 16, 18) were used to select representative artificial receptors for each target. FIGS. 29, 30 and 31 employ data obtained in the present example to illustrate identification of each of these three targets by their distinctive binding patterns.

Conclusions

The optimum receptor for a particular target requires molecular recognition which is greater than the expected sum of the individual hydrophilic, hydrophobic, ionic, etc. interactions. Thus, the identification of an optimum (specific, sensitive) artificial receptor from the limited pool of candidate receptors explored in this prototype study, was not expected and not likely. Rather, the goal was to demonstrate that all of the key components of the CARA: Combinatorial Artificial Receptor Array concept could be assembled to form a functional receptor microarray. This goal has been successfully demonstrated.

This study has conclusively established that CARA microarrays can be readily prepared and that target binding to the candidate receptor environments can be used to identify artificial receptors and test ligands. In addition, these results demonstrate that there is significant binding enhancement for the building block heterogeneous (n=2, n=3, or n=4) candidate receptors when compared to their homogeneous (n=1) counterparts. When combined with the binding pattern recognition results and the demonstrated importance of both the heterogeneous receptor elements and heterogeneous building blocks, these results clearly demonstrate the significance of the CARA Candidate Artificial Receptor->Lead Artificial Receptor->Working Artificial Receptor strategy.

### Example 3

### Preparation and Evaluation of Microarrays of Candidate Artificial Receptors Including Reversibly Immobilized Building Blocks

Microarrays of candidate artificial receptors including building blocks immobilized through van der Waals interactions were made and evaluated for binding of a protein ligand. The evaluation was conducted at several temperatures, above and below a phase transition temperature for the lawn (vide infra).

Materials and Methods

Building blocks 2-2,2-4, 2-6,4-2, 4-4, 4-6, 6-2, 6-4, 6-6 where prepared as described in Example 1. The C12 amide was prepared using the previously described carbodiimide activation of the carboxyl followed by addition of dodecylamine.

Amino lawn microarray plates (Telechem) were modified to produce the C18 lawn by reaction of stearoyl chloride (Aldrich Chemical Co.) in A) dimethylformamide/PEG 400 solution (90:10, v/v, PEG 400 is polyethylene glycol average MW 400 (Aldrich Chemical Co.) or B) methylene chloride/TEA solution (100 ml methylene chloride, 200 ul triethylamine) using the lawn modification procedures generally described in Example 2.

The C18 lawn plates where printed using the SpotBot standard procedure as described in Example 2. The building blocks were in printing solutions prepared by solution of ca. 10 mg of each building block in 300 ul of methylene chloride and 100 ul methanol. To this stock was added 900 ul of dimethylformamide and 100 ul of PEG 400. The 36 combinations of the 9 building blocks taken two at a time (N9:n2, 36 combinations) where prepared in a 384-well microwell plate which was then used in the SpotBot to print the microarray in quadruplicate. A random selection of the print positions contained only print solution.

The selected microarray was incubated with a 1.0 μg/ml solution of the probe protein (e.g. fluorescently labeled cholera toxin B) using the following variables: the microarray was washed with methylene chloride, ethanol and water to create a control plate, the microarray was incubated at 4° C., 23° C., or 44° C. After incubation, the plate(s) were rinsed with water, dried and scanned (AXON 4100A). Data analysis was as described in Example 2.

Results

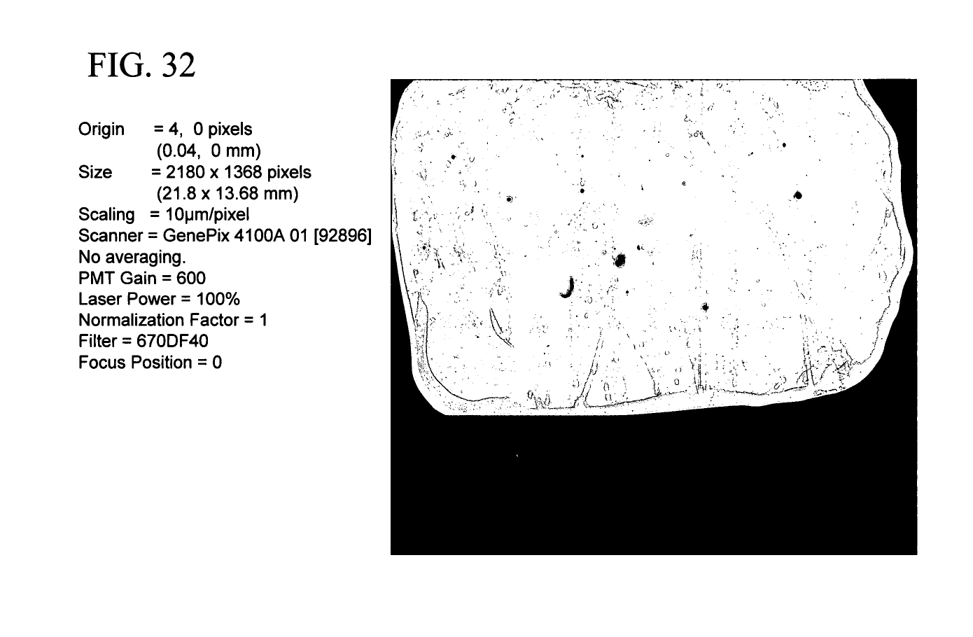

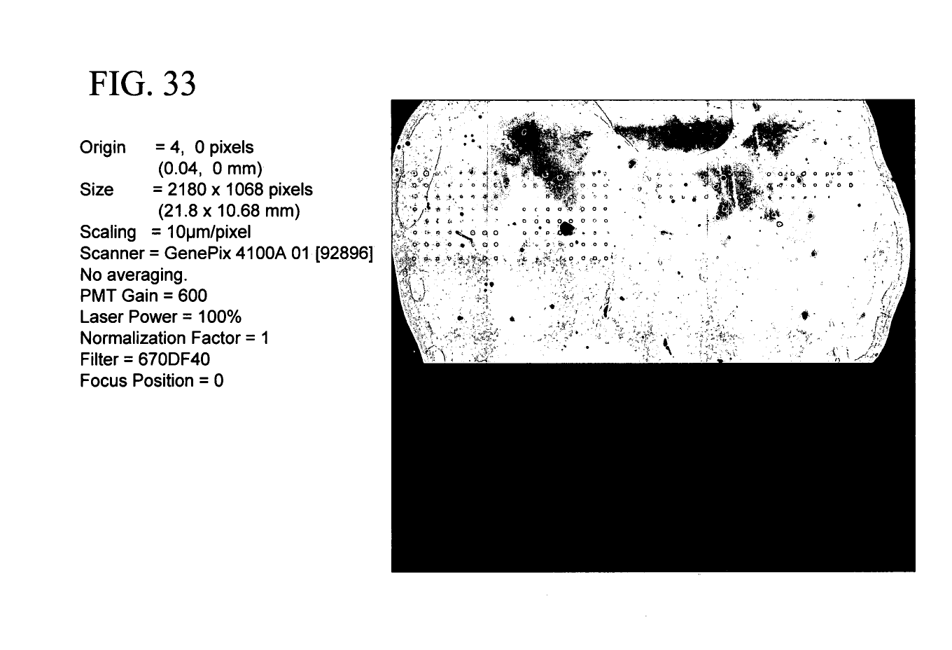

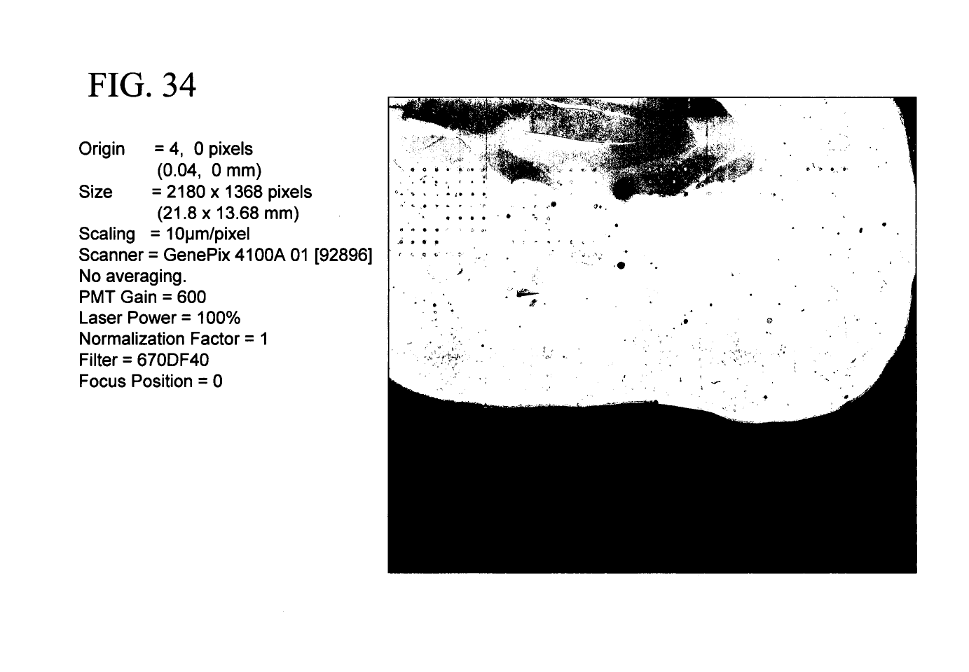

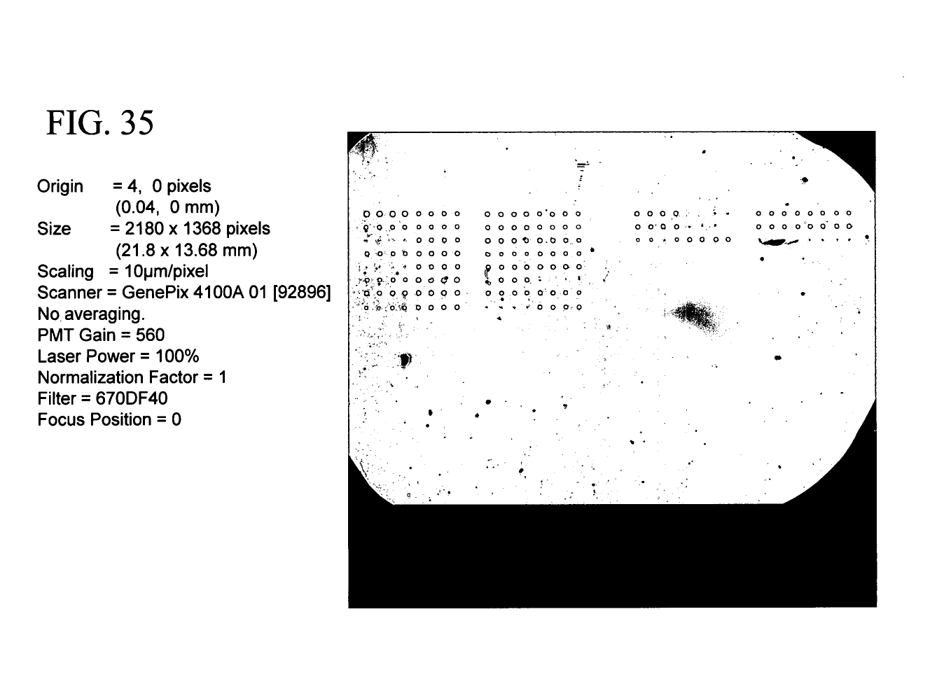

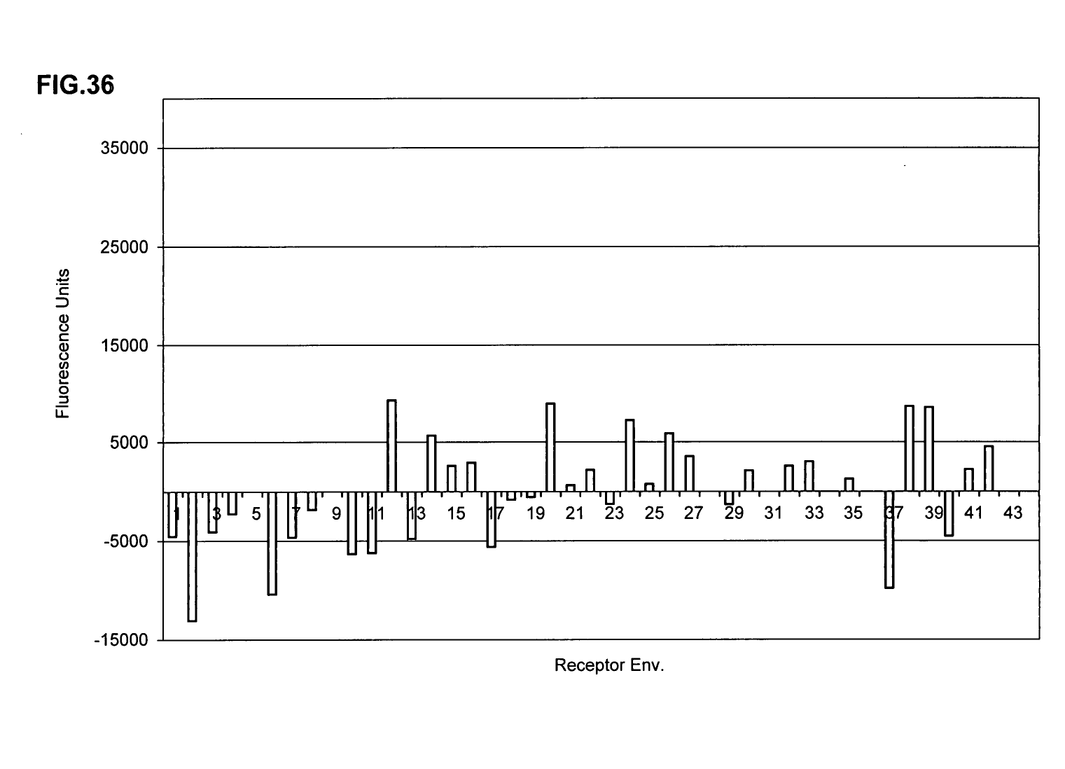

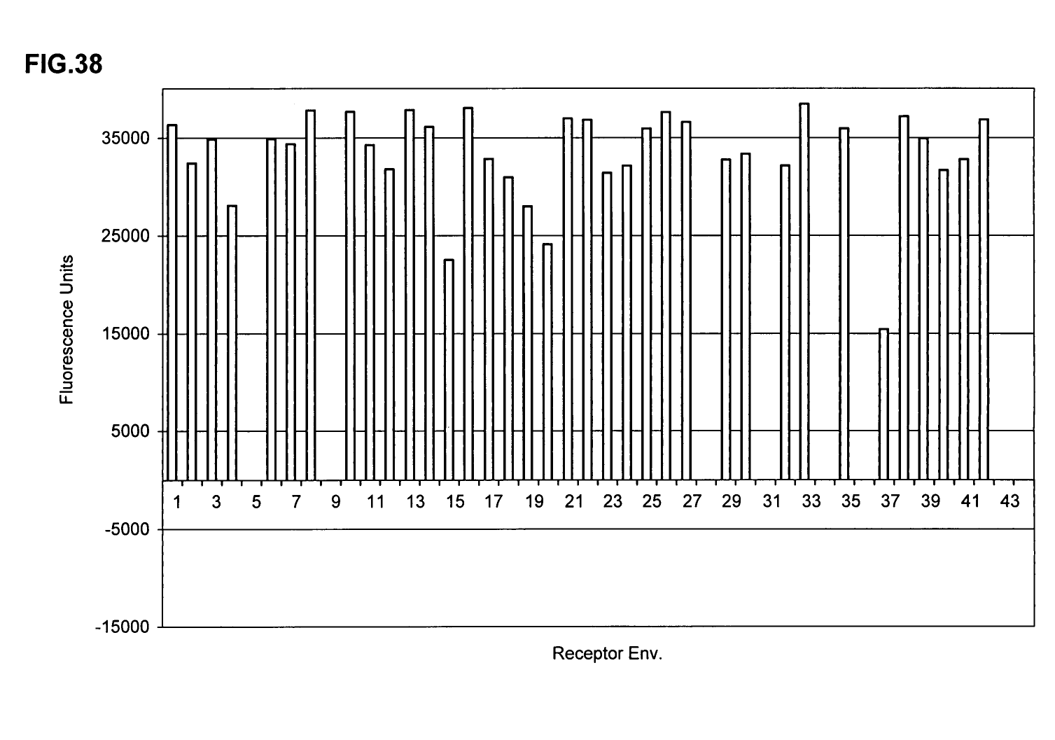

A control array from which the building blocks had been removed by washing with organic solvent did not bind cholera toxin (FIG. 32). FIGS. 33-35 illustrate fluorescence signals from arrays printed identically, but incubated with cholera toxin at 4° C., 23° C., or 44° C., respectively. Spots of fluorescence can be seen in each array, with very pronounced spots produced by incubation at 44° C. The fluorescence values for the spots in each of these three arrays are shown in FIGS. 36-38. Fluorescence signal generally increases with temperature, with many nearly equally large signals observed after incubation at 44° C. Linear increases with temperature can reflect expected improvements in binding with temperature. Nonlinear increases reflect rearrangement of the building blocks on the surface to achieve improved binding, which occurred above the phase transition for the lipid surface (vide infra).

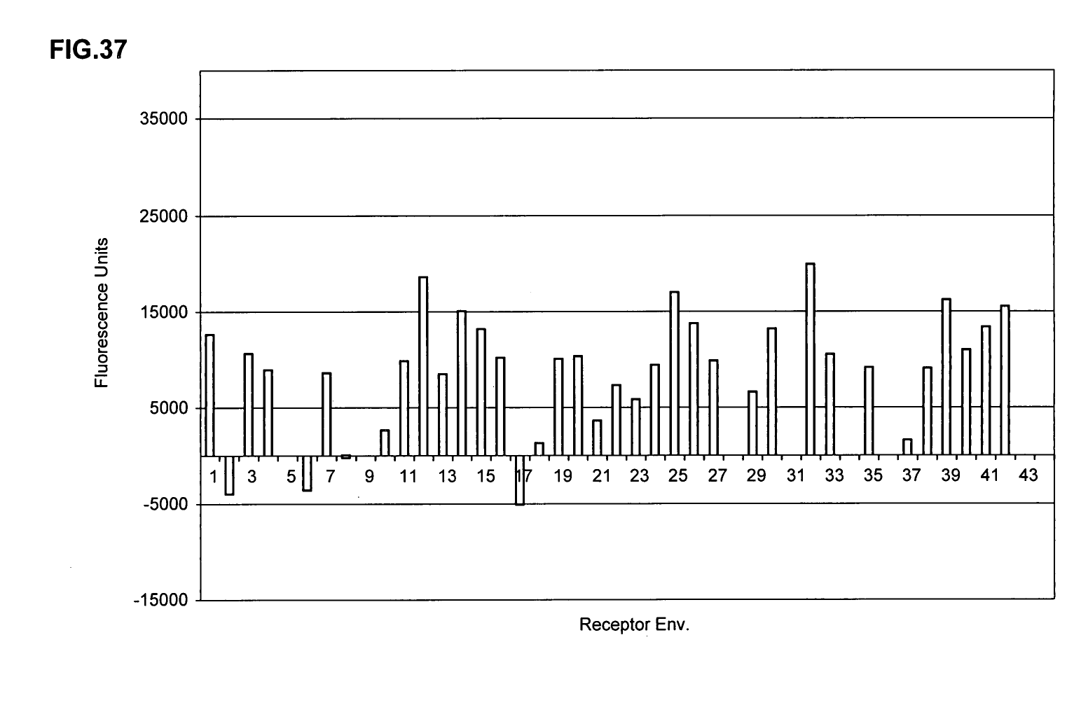

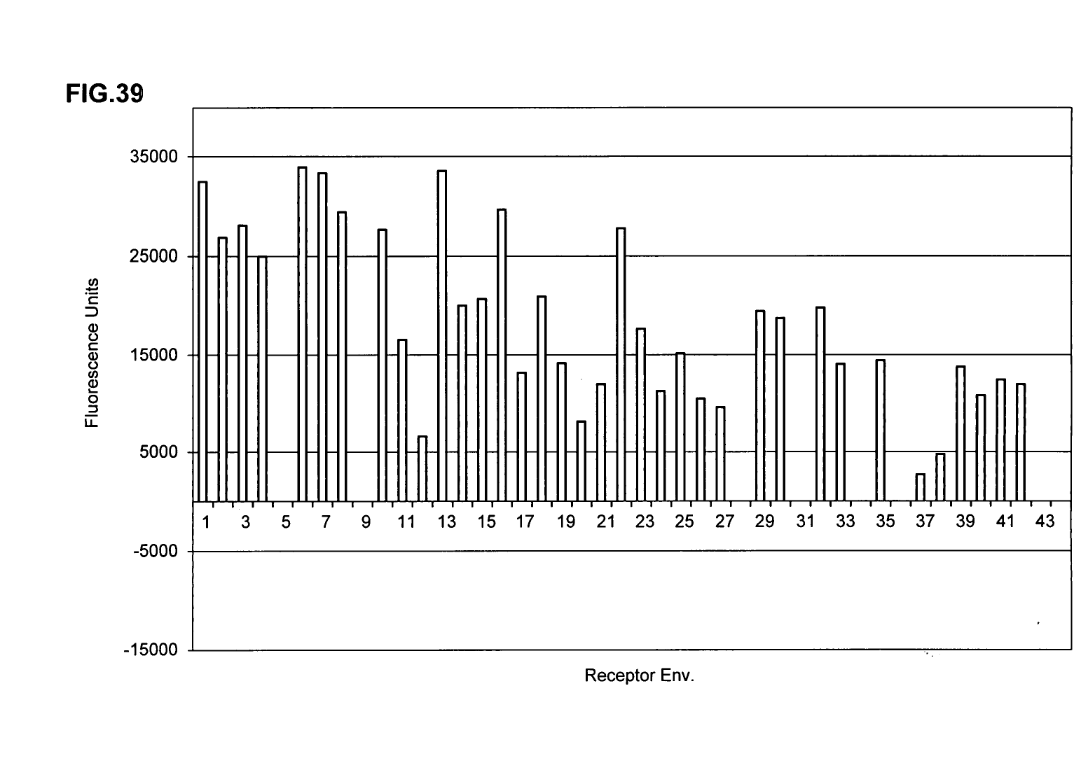

FIG. 39 can be compared to FIG. 37. The fluorescence signals plotted in FIG. 37 resulted from binding to reversibly immobilized building blocks on a support at 23° C. The fluorescence signals plotted in FIG. 39 resulted from binding to covalently immobilized building blocks on a support at 23° C. These figures compare the same combinations of building blocks in the same relative positions, but immobilized in two different ways.

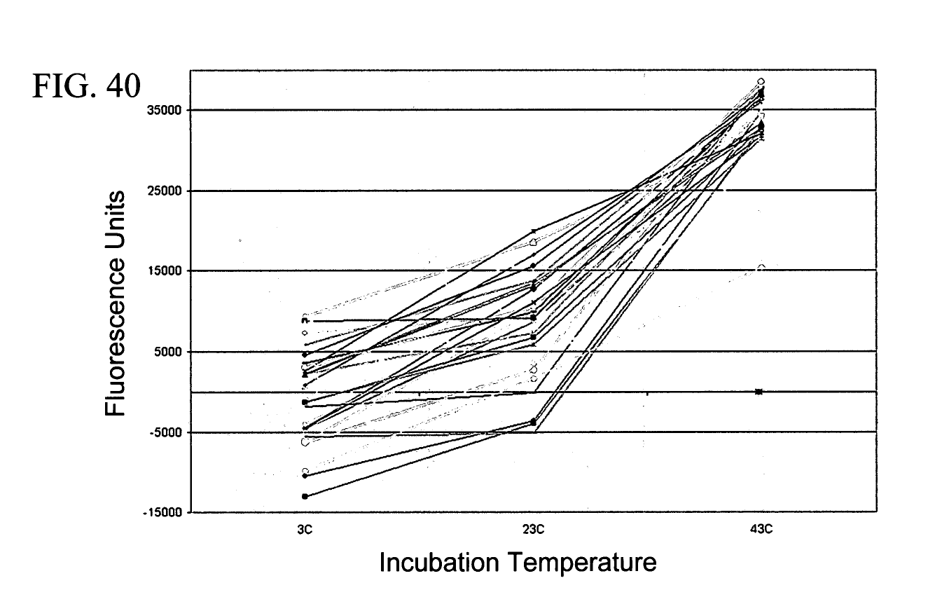

FIG. 40 illustrates the changes in fluorescence signal from individual combinations of building blocks at 4° C., 23° C., or 44° C. This graph illustrates that at least one combination of building blocks (candidate artificial receptor) exhibited a signal that remained constant as temperature increased. At least one candidate artificial receptor exhibited an approximately linear increase in signal as temperature increased. Such a linear increase indicates normal temperature effects on binding. The candidate artificial receptor with the lowest binding signal at 4° C. became one of the best binders at 44° C. This indicates that rearrangement of the building blocks of this receptor above the phase transition for the lipophilic lawn produced increased binding. Other receptors characterized by greater changes in binding between 23° C. and 44° C. (compared to between 4° C. and 23° C.) also underwent dynamic affinity optimization.

Conclusions

This experiment demonstrated that an array including reversibly immobilized building blocks binds a protein substrate, like an array with covalently immobilized building blocks. The binding increased nonlinearly as temperature increased, indicating that movement of the building blocks increased binding. The candidate artificial receptors demonstrated improved binding upon mobilization of the building blocks.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration to. The phrase “adapted and configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A method of making a heterogeneous building block array, the method comprising:
forming a plurality of spots on a solid support, the spots comprising a plurality of building blocks; and
immobilizing building blocks to the support in the spots by covalent coupling, by an ionic interaction, or by a combination thereof.

2. The method of claim 1, wherein immobilizing comprises covalent coupling.

3. The method of claim 2, wherein:
the support comprises an amine nitrogen and the building block comprises a carbonyl carbon;
the support comprises a carbonyl carbon and building block comprises an amine nitrogen;
or combination thereof.

4. The method of claim 1, wherein immobilizing comprises ionic interaction.

5. The method of claim 4, wherein:
the support comprises a carboxylate and the building block comprises an ammonium;
the support comprises an ammonium and the building block comprises a carboxylate;
or combination thereof.

6. The method of claim 4, wherein the support comprises amine, quaternary ammonium, ferrocene, or mixture thereof.

7. The method of claim 4, wherein the support comprises carboxylate, phenol substituted with strongly electron withdrawing group, phosphate, phosphonate phosphinate, sulphate, sulphonates, thiocarboxylate, hydroxamic acid, or mixture thereof.

8. The method of claim 4, wherein the building block comprises amine, quaternary ammonium, ferrocene, or mixture thereof.

9. The method of claim 4, wherein the building block comprises carboxylate, phenol substituted with strongly electron withdrawing group, phosphate, phosphonate phosphinate, sulphate, sulphonates, thiocarboxylate, hydroxamic acid, or mixture thereof.

10. The method of claim 1, further comprising mixing a plurality of building blocks and employing the mixture in forming the plurality of spots.

11. The method of claim 1, wherein the solid support comprises a glass plate or microscope slide.

12. A method of making a receptor surface, the method comprising:
forming a region on a solid support, the region comprising a plurality of building blocks; and
immobilizing building blocks to the support in the spots by covalent coupling, by an ionic interaction, or by a combination thereof.

13. The method of claim 12, further comprising mixing a plurality of building blocks and employing the mixture in forming the receptor surface.

14. A method of making an artificial receptor, the method comprising:
forming a region on a support, the region comprising a plurality of building blocks;
coupling building blocks to the support in the region by covalent coupling, by an ionic interaction, or by a combination thereof.

15. The method of claim 14, wherein the region is a spot.

16. A composition comprising:
a support; and
a portion of the support comprising a plurality of building blocks;
building blocks being immobilized on the support by covalent coupling, by an ionic interaction, or by a combination thereof.

17. The composition of claim 16, comprising building blocks immobilized by covalent coupling.

18. The composition of claim 17, comprising building blocks immobilized by acetal linkage, ketal linkage, disulfide linkage, ester linkage, or combination thereof.

19. The composition of claim 17, wherein:
the support comprises an amine nitrogen and the building blocks comprise a carbonyl carbon;
the support comprises a carbonyl carbon and the building blocks comprise an amine nitrogen;
or combination thereof.

20. The composition of claim 16, comprising building blocks immobilized by ionic interaction.

21. The composition of claim 20, wherein:
the support comprises a carboxylate and the building blocks comprise an ammonium;
the support comprises an ammonium and the building blocks comprise a carboxylate;
or combination thereof.

22. The composition of claim 20, wherein the support comprises amine, quaternary ammonium, ferrocene, or mixture thereof.

23. The composition of claim 20, wherein the support comprises carboxylate, phenol substituted with strongly electron withdrawing group, phosphate, phosphonate phosphinate, sulphate, sulphonates, thiocarboxylate, hydroxamic acid, or mixture thereof.

24. The composition of claim 20, wherein the building block comprises amine, quaternary ammonium, ferrocene, or mixture thereof.

25. The composition of claim 20, wherein the building block comprises carboxylate, phenol substituted with strongly electron withdrawing group, phosphate, phosphonate phosphinate, sulphate, sulphonates, thiocarboxylate, hydroxamic acid, or mixture thereof.

26. The composition of claim 16, comprising a candidate artificial receptor, a lead artificial receptor, a working artificial receptor, or a combination thereof.

27. The composition of claim 24, wherein the artificial receptor comprises 2, 3, 4, 5, or 6 different building blocks.

28. The composition of claim 16, comprising a plurality of spots on the support;
the spots comprising a plurality of building blocks; and
the building blocks being coupled to the support.

29. The composition of claim 23, wherein the spots are configured in an array.

30. The composition of claim 29, wherein the array comprises more than 1 million spots.

31. The composition of claim 28, wherein the spots comprise 2, 3, 4, 5, or 6 building blocks.

32. The composition of claim 28, wherein the support comprises a solid support.

33. The composition of claim 32, comprising a plurality of spots on a surface of the solid support.

34. The composition of claim 28, comprising a functionalized lawn coupled to the support and the building blocks immobilized in spots to the lawn.

35. The composition of claim 34, comprising building blocks immobilized by covalent coupling.

36. The composition of claim 35, comprising building blocks immobilized by acetal linkage, ketal linkage, disulfide linkage, ester linkage, or combination thereof.

37. The composition of claim 35, wherein:
the functionalized lawn comprises an amine nitrogen and the building blocks comprise a carbonyl carbon;
the functionalized lawn comprises a carbonyl carbon and the building blocks comprise an amine nitrogen;
or combination thereof.

38. The composition of claim 34, comprising building blocks immobilized by ionic interaction.

39. The composition of claim 38, wherein:
the functionalized lawn comprises a carboxylate and the building blocks comprise an ammonium;
the functionalized lawn comprises an ammonium and the building blocks comprise a carboxylate;
or combination thereof.

40. The composition of claim 38, wherein the functionalized lawn comprises amine, quaternary ammonium, ferrocene, or mixture thereof.

41. The composition of claim 38, wherein the functionalized lawn comprises carboxylate, phenol substituted with strongly electron withdrawing group, phosphate, phosphonate phosphinate, sulphate, sulphonates, thiocarboxylate, hydroxamic acid, or mixture thereof.

42. The composition of claim 38, wherein the building block comprises amine, quaternary ammonium, ferrocene, or mixture thereof.

43. The composition of claim 38, wherein the building block comprises carboxylate, phenol substituted with strongly electron withdrawing group, phosphate, phosphonate phosphinate, sulphate, sulphonates, thiocarboxylate, hydroxamic acid, or mixture thereof.

44. The composition of claim 43, comprising a functionalized glass support.

45. The composition of claim 43, wherein:
the support comprises a surface;
the surface comprises a region; and
the region comprises a plurality of building blocks;
the building blocks being coupled to the support.

46. The composition of claim 45, wherein the support comprises a tube or well.

47. The composition of claim 45, further comprising a functionalized lawn coupled to the tube or well and the building blocks immobilized to the lawn.

48. A heterogeneous building block array comprising:
a support; and
a plurality of spots on the support;
the spots comprising a plurality of building blocks; and
building blocks being immobilized on the support by covalent coupling, by an ionic interaction, or by a combination thereof.

49. A composition comprising:
a surface; and
a region on the surface comprising a plurality of building blocks;
building blocks being immobilized on the support by covalent coupling, by an ionic interaction, or by a combination thereof.

50. A composition comprising:
a support; and
a portion of the support comprising a plurality of building blocks;
building blocks being immobilized on the support by covalent coupling, by an ionic interaction, by hydrophobic interaction, or by a combination thereof.

51. The composition of claim 50, comprising building blocks immobilized by hydrophobic interaction.

52. The composition of claim 51, wherein the support and building blocks comprise independently branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof.

53. The composition of claim 50, comprising building blocks immobilized by hydrophobic interaction and by covalent coupling.

54. The composition of claim 53, comprising building blocks immobilized by hydrophobic interaction; and acetal linkage, ketal linkage, disulfide linkage, ester linkage, or combination thereof.

55. The composition of claim 53, wherein the support comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and carbonyl carbon, amine nitrogen, thiol, alcohol, carboxyl group, or combination thereof.

56. The composition of claim 53, wherein the building block comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and carbonyl carbon, amine nitrogen, thiol, alcohol, carboxyl group, or combination thereof.

57. The composition of claim 50, comprising building blocks immobilized by hydrophobic interaction and by ionic interaction.

58. The composition of claim 57, wherein the support comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and positively charged moiety.

59. The composition of claim 58, wherein the building block comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and negatively charged moiety.

60. The composition of claim 57, wherein the support comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and negatively charged moiety.

61. The composition of claim 60, wherein the building block comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and positively charged moiety.

62. The composition of claim 50, comprising a functionalized lawn coupled to the support and the building blocks immobilized in spots to the lawn.

63. The composition of claim 62, comprising building blocks immobilized by hydrophobic interaction.

64. The composition of claim 63, wherein the lawn and building blocks comprise independently branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof.

65. The composition of claim 62, comprising building blocks immobilized by hydrophobic interaction and by covalent coupling.

66. The composition of claim 65, comprising building blocks immobilized by hydrophobic interaction; and acetal linkage, ketal linkage, disulfide linkage, ester linkage, or combination thereof.

67. The composition of claim 65, wherein the lawn comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and carbonyl carbon, amine nitrogen, thiol, alcohol, carboxyl group, or combination thereof.

68. The composition of claim 65, wherein the building block comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and carbonyl carbon, amine nitrogen, thiol, alcohol, carboxyl group, or combination thereof.

69. The composition of claim 62, comprising building blocks immobilized by hydrophobic interaction and by ionic interaction.

70. The composition of claim 69, wherein the lawn comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and positively charged moiety.

71. The composition of claim 70, wherein the building block comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and negatively charged moiety.

72. The composition of claim 69, wherein the lawn comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and negatively charged moiety.

73. The composition of claim 72, wherein the building block comprises branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof; and positively charged moiety.

74. An article of manufacture comprising:
a support, a functionalized lawn reagent, and a plurality of building blocks;
the functionalized lawn being configured to be coupled to the support;
the plurality of building blocks being configured to be immobilized to the lawn by covalent coupling, by an ionic interaction, or by a combination thereof.

75. The article of manufacture of claim 74, wherein the functionalized lawn reagent comprises a first covalent bonding moiety and the building block comprises a second covalent bonding moiety.

76. The article of manufacture of claim 74, wherein the functionalized lawn reagent comprises a first charged moiety and the building block comprises a second charged moiety, the first and second charged moieties having opposite charges.

77. The article of manufacture of claim 74, comprising a functionalized glass support.

78. An article of manufacture comprising:
a support, a functionalized lawn reagent, and a plurality of building blocks;
the functionalized lawn being configured to be coupled to the support;
the plurality of building blocks being configured to be immobilized to the lawn by covalent coupling, by an ionic interaction, by hydrophobic interaction, or by a combination thereof.

79. The article of manufacture of claim 78, wherein the functionalized lawn reagent comprises a first lipophilic moiety and the building block comprises a second lipophilic moiety.

80. A method of making a heterogeneous building block array, the method comprising:
forming a plurality of spots on a solid support, the spots comprising a plurality of building blocks; and
immobilizing building blocks to the support in the spots by covalent coupling, by an ionic interaction, hydrophobic interaction, or by a combination thereof.

81. The method of claim 80, comprising immobilizing building blocks by hydrophobic interaction.

82. The method of claim 81, wherein the support and building blocks comprise independently branched or straight chain, substituted or unsubstituted C₆₋₃₆ alkyl; branched or straight chain, substituted or unsubstituted C₆₋₃₆ alkenyl with 1 to 4 double bonds; branched or straight chain, substituted or unsubstituted C₆₋₃₆ alkynyl with 1 to 4 triple bonds; branched or straight chain, substituted or unsubstituted C₆₋₃₆ arylalkyl; branched or straight chain, substituted or unsubstituted C₆₋₃₆ arylalkenyl with 1 to 4 double bonds; branched or straight chain, substituted or unsubstituted C₆₋₃₆ arylalkynyl with 1 to 4 triple bonds; polyaromatic hydrocarbon; substituted or unsubstituted cycloalkane; or mixtures thereof.
